SEQUENCE LISTING (25 sequences)

SEQ ID NO: 1 - protein, 5 aa, Synthetic construct
DYLVH

SEQ ID NO: 2 - protein, 17 aa, Synthetic construct
WINTYTGTPTYADDFEG

SEQ ID NO: 3 - protein, 13 aa, Synthetic construct
SWRRGLRGLGFDY

SEQ ID NO: 4 - protein, 13 aa, Synthetic construct
SWRRGLRGIGFDY

SEQ ID NO: 5 - protein, 13 aa, Synthetic construct
SWRRGIRGLGFDY

SEQ ID NO: 6 - protein, 13 aa, Synthetic construct
SWRRGIRGIGFDY

SEQ ID NO: 7 - protein, 11 aa, Synthetic construct
KASKSINKYLA

SEQ ID NO: 8 - protein, 7 aa, Synthetic construct
DGSTLQS

SEQ ID NO: 9 - protein, 9 aa, Synthetic construct
QQHNEYPPT

SEQ ID NO: 10 - protein, 122 aa, Synthetic construct
QIQLVQSGPELREPGESVKLSCKASGYTFTDYLVHWVKQAPGKGLKWMGWINTYTGTPTYADDFEGRFVFSLEASASTANLQISNLKNEDTATYFCARSWRRGLRGLGFDYWGQGVFVTVSS

SEQ ID NO: 11 - protein, 122 aa, Synthetic construct
QIQLVQSGPELREPGESVKLSCKASGYTFTDYLVHWVKQAPGKGLKWMGWINTYTGTPTYADDFEGRFVFSLEASASTANLQISNLKNEDTATYFCARSWRRGLRGIGFDYWGQGVFVTVSS

SEQ ID NO: 12 - protein, 122 aa, Synthetic construct
QIQLVQSGPELREPGESVKLSCKASGYTFTDYLVHWVKQAPGKGLKWMGWINTYTGTPTYADDFEGRFVFSLEASASTANLQISNLKNEDTATYFCARSWRRGIRGLGFDYWGQGVFVTVSS

SEQ ID NO: 13 - protein, 122 aa, Synthetic construct
QIQLVQSGPELREPGESVKLSCKASGYTFTDYLVHWVKQAPGKGLKWMGWINTYTGTPTYADDFEGRFVFSLEASASTANLQISNLKNEDTATYFCARSWRRGIRGIGFDYWGQGVFVTVSS

SEQ ID NO: 14 - protein, 122 aa, Synthetic construct
QVQLVQSGSELKKPGESVKVSCKASGYTFTDYLVHWVRQAPGQGLEWMGWINTYTGTPTYADDFEGRFVFSLDTSVSTANLQISSLKAEDTAVYFCARSWRRGLRGIGFDYWGQGTLVTVSS

SEQ ID NO: 15 - protein, 122 aa, Synthetic construct
QVQLVQSGPELKKPGESVKVSCKASGYTFTDYLVHWVRQAPGKGLEWMGWINTYTGTPTYADDFEGRFVFSLDTSASTANLQICSLKNEDTATYYCARSWRRGLRGIGFDYWGQGTLVTVSS

SEQ ID NO: 16 - protein, 107 aa, Synthetic construct
DVQMTQSPYNLAASPGESVSINCKASKSINKYLAWYQQKPGKPNKLLIYDGSTLQSGIPSRFSGSGSGTDFTLTIRGLEPEDFGLYYCQQHNEYPPTFGAGTKLELK

SEQ ID NO: 17 - protein, 107 aa, Synthetic construct
DIQMTQSPYSLSASVGDRVTITCKASKSINKYLAWFQQKPGKPNKLLIYDGSTLQSGVPSRFSGSGSGTDFTLTISSLEPEDFATYYCQQHNEYPPTFGQGTKLEIK

SEQ ID NO: 18 - protein, 452 aa, Synthetic construct
QIQLVQSGPELREPGESVKLSCKASGYTFTDYLVHWVKQAPGKGLKWMGWINTYTGTPTYADDFEGRFVFSLEASASTANLQISNLKNEDTATYFCARSWRRGLRGLGFDYWGQGVFVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 452 aa, Synthetic construct
QIQLVQSGPELREPGESVKLSCKASGYTFTDYLVHWVKQAPGKGLKWMGWINTYTGTPTYADDFEGRFVFSLEASASTANLQISNLKNEDTATYFCARSWRRGLRGIGFDYWGQGVFVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 452 aa, Synthetic construct
QIQLVQSGPELREPGESVKLSCKASGYTFTDYLVHWVKQAPGKGLKWMGWINTYTGTPTYADDFEGRFVFSLEASASTANLQISNLKNEDTATYFCARSWRRGIRGLGFDYWGQGVFVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 452 aa, Synthetic construct
QIQLVQSGPELREPGESVKLSCKASGYTFTDYLVHWVKQAPGKGLKWMGWINTYTGTPTYADDFEGRFVFSLEASASTANLQISNLKNEDTATYFCARSWRRGIRGIGFDYWGQGVFVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 452 aa, Synthetic construct
QVQLVQSGSELKKPGESVKVSCKASGYTFTDYLVHWVRQAPGQGLEWMGWINTYTGTPTYADDFEGRFVFSLDTSVSTANLQISSLKAEDTAVYFCARSWRRGLRGIGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 452 aa, Synthetic construct
QVQLVQSGPELKKPGESVKVSCKASGYTFTDYLVHWVRQAPGKGLEWMGWINTYTGTPTYADDFEGRFVFSLDTSASTANLQICSLKNEDTATYYCARSWRRGLRGIGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 214 aa, Synthetic construct
DVQMTQSPYNLAASPGESVSINCKASKSINKYLAWYQQKPGKPNKLLIYDGSTLQSGIPSRFSGSGSGTDFTLTIRGLEPEDFGLYYCQQHNEYPPTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 214 aa, Synthetic construct
DIQMTQSPYSLSASVGDRVTITCKASKSINKYLAWFQQKPGKPNKLLIYDGSTLQSGVPSRFSGSGSGTDFTLTISSLEPEDFATYYCQQHNEYPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0346667 A1
(43) Pub. Date: Nov. 13, 2025

(54) PHARMACEUTICAL COMPOSITION OF ANTI-TRBV9 ANTIBODY AND USE THEREOF

(71) Applicant: JOINT STOCK COMPANY «BIOCAD», Saint Petersburg (RU)

(72) Inventors: Fedor Fandatovich NIDZVEDSKII, Ekaterinburg (RU); Ekaterina Vladislavovna OVCHARENKO, Podolsk (RU); Aleksandra Aleksandrovna SOZONOVA, Saint Petersburg (RU); Alina Aleksandrovna KOSTANDIAN, Saint Petersburg (RU); Anastasiia Alekseevna ANDREEVA, Saratov (RU); Ekaterina Aleksandrovna LOMKOVA, Saint Petersburg (RU); Aleksandr Olegovich IAKOVLEV, Moscow (RU); Dmitry Valentinovich MOROZOV, Saint Petersburg (RU)

(21) Appl. No.: 18/834,263

(22) PCT Filed: Jan. 31, 2023
(86) PCT No.: PCT/RU2023/050012

§ 371 Date: Jul. 30, 2024

(30) Foreign Application Priority Data
Jan. 31, 2022 (RU) 2022102193

(51) Int. Cl. C07K 16/28; A61K 47/54

(52) U.S. Cl. CPC C07K 16/2809; A61K 47/542; A61K 47/545

(57) ABSTRACT

The invention relates to the field of pharmacy and medicine, specifically to pharmaceutical compositions of anti-TRBV9 antibody. The invention further relates to the use of said compositions for treating a disease or disorder mediated by T-lymphocytes bearing a TRBV9 segment within the T cell receptor, in a subject in need thereof.

### FIELD OF THE INVENTION

The present invention relates to the field of pharmacy and medicine, specifically to pharmaceutical compositions of anti-TRBV9 antibody that may be used to treat a disease or disorder mediated by T-lymphocytes bearing a TRBV9 segment within the T cell receptor.

### BACKGROUND OF THE INVENTION

Autoimmune diseases are caused by autoreactive T-lymphocytes (Haroon N et al., Arthritis Rheum. 2013 October; 65(10):2645-54., Duarte J. et al., PloS One 2010 May 10; 5(5):e10558; Konig M. et al., Front Immunol 2016 Jan. 25; 7:11). The major role in the appearance of autoreactive T-lymphocyte clones is played by the interaction of the antigen-recognizing T cell receptor (TCR) with proteins of the major histocompatibility complex (MHC, HLA), which present on their surface the processed peptides of intracellular proteins or those of proteins of pathogenic organisms. A number of autoimmune diseases are associated with the presence of a certain variant of the HLA gene in humans. Accordingly, the HLA-B27 allele is associated with ankylosing spondylitis, reactive arthritis, and Crohn's disease. The risk of developing autoimmune diseases in carriers of certain HLA allelic variants can be explained by the fact that these alleles present preferentially certain peptides that are autoantigens, an immune response against which triggers the development of an autoimmune disease. One of the possible mechanisms underlying an autoimmune reaction is presenting, by the histocompatibility complex molecules, the peptides from proteins of bacterial or viral origin, which are homologous to the organism's own peptides, which fact may lead to an immune response against self antigens as a result of cross-reactivity.

The prior art provides that a T cell receptor (TCR) sequence is a marker allowing to identify a T-lymphocyte clone involved in the pathogenesis of an autoimmune disease. T cell receptor subunits belong structurally to the immunoglobulin superfamily and are formed from several gene segments. The variable regions of TCR form the antigen-binding center of TCR. This means that they are clone-specific, that is, they are different in those T-lymphocytes that react to different antigens.

T lymphocytes (T cells) are stimulated when antigens bind to T cell receptors (TCRs) thereof. The TCR, a defining structure of T cells, is a transmembrane heterodimer consisting of either an alpha and beta chain or delta and gamma chain linked by a disulphide bond. Within these chains there are complementary determining regions (CDRs) which determine the antigen to which the TCR will bind. TCR development occurs through a lymphocyte specific process of gene recombination, which assembles a final sequence from a large number of potential segments. This genetic recombination of TCR gene segments in somatic T cells occurs during the early stages of development in the thymus. The TCRα gene locus contains variable (V) and joining (J) gene segments (Vβ and Jβ), whereas the TCRβ locus contains a D gene segment in addition to Vα and Jα segments. Accordingly, the α chain is generated from VJ recombination and the β chain is involved in VDJ recombination.

The TCR α chain gene locus consists of 46 variable segments (TRAV), 8 joining segments (TRAJ) and the constant region. The TCR β chain gene locus consists of 48 variable segments (TRBV) followed by two diversity segments (TRBD), 12 joining segments (TRBJ) and two constant regions. (Bio-Rad. Mini-review|An overview of T cell receptors [Electronic resource]//Bio-Rad. URL: https://www.bio-rad-antibodies.com/t-cell-receptor-minireview.html (accessed: 24.04.2020)).

To date, a significant amount of data has been accumulated demonstrating that the development of HLA-B27-associated diseases is due to the expansion of antigen-specific T-lymphocyte clones.

The consensus variant of autoimmune TCRs has been described in ankylosing spondylitis (radiographic axial spondyloarthritis); it has been shown that it is present in synovial fluid and peripheral blood in patients with ankylosing spondylitis and is absent with the same analysis depth in healthy donors, regardless of the HLA*B27 allele status (Faham M. et al., Arthritis Rheumatol. 2017; 69(4):774-784; Komech E et al. 12th EJI-EFIS Tatra Immunology Conference; 2016 Sep. 3-7; Strbske Pleso, Slovakia. Abstract book p. 39). Said TCRs belong to the TRBV9 family (according to the IMGT nomenclature).

It has been shown that T cell receptors bearing beta chains of the TRBV9 family are also involved in the development of such an autoimmune disease as celiac disease (Petersen J et al., J Immunol. 2015; 194(12): 6112-22). They are also found on the surface of T cells subject to malignization in T cell lymphomas and T cell leukemias, including T cell lymphoma caused by the Epstein-Barr virus (EBV) (Toyabe S et al., Clin Exp Immunol. 2003; 134(1): 92-97).

Considering the above, a TRBV9 protein may serve as a target for a cytotoxic monoclonal antibody that will induce depletion of TRBV9+T-lymphocytes (TRBV9-positive T-lymphocytes), including pathogenic autoreactive T-lymphocyte clones.

Monoclonal anti-TRBV9 antibodies are known from the prior art: WO2019/132738, WO2020/139171, WO2020/091635, WO2020/139175. The prior art also provides a pharmaceutical composition of anti-TRBV9 antibody comprising a citrate buffer (WO2020/139171). However, it has been found by the authors of the invention that the anti-TRBV9 antibody in a citrate buffer solution tends to aggregate, therefore the composition of anti-TRBV9 antibody comprising a citrate buffer will not be stable.

In connection with the above, there is a need in developing stable pharmaceutical compositions of anti-TRBV9 antibody that may be used as a medicinal product for treating a disease or disorder mediated by the human T cell receptor bearing TRBV9.

### DETAILED DESCRIPTION OF THE INVENTION

### Definitions

Unless defined otherwise herein, all technical and scientific terms used in connection with the present invention will have the same meaning as is commonly understood by those skilled in the art.

Furthermore, unless otherwise required by context, singular terms shall include plural terms, and the plural terms shall include the singular terms.

As used in the present description and claims that follow, unless otherwise dictated by the context, the words “have”, “include,” and “comprise” or variations thereof such as “has”, “having,” “includes”, “including”, “comprises,” or “comprising,” will be understood to imply the inclusion of a stated integer or group of integers but not the exclusion of any other integer or group of integers.

The term “antibody” or “immunoglobulin” (Ig) includes full-length antibodies or any antigen binding fragment (i.e, “antigen-binding portion”) or individual chains thereof. The term “antibody” within the scope of the present invention is used in the broadest sense and may include, without limitation, monoclonal antibodies (including full-length monoclonal antibodies), polyclonal antibodies, humanized, fully human antibodies and chimeric antibodies.

A full-length antibody refers to a glycoprotein comprising at least two heavy (H) chains and two light (L) chains interconnected by disulfide bonds. Each heavy chain comprises a heavy chain variable region (abbreviated referred to in the present description as VH) and a heavy chain constant region. The constant region is identical in all antibodies of the same isotype, but differs in antibodies of different isotypes. Heavy chains γ, α and δ have a constant region composed of three constant domains CH1, CH2 and CH3 (in a line), and a hinge region for added flexibility (Woof J., Burton D., Nat Rev Immunol 4, 2004, pp. 89-99). In mammals, known are only two types of light chains denoted by lambda (λ) and kappa (κ). Each light chain consists of a light chain variable region (abbreviated referred to in the present description as VL) and light chain constant region. The approximate length of a light chain is 211 to 217 amino acids. Preferably, the light chain is a lambda (A) light chain, and the constant domain CL is preferably C lambda (A).

VL and VH regions may be further subdivided into hyper-variability regions called complementarity determining regions (CDRs), located between regions that are more conserved, termed framework regions (FRs). Each VH and VL is composed of three CDRs and four FRs, arranged from amino-terminus to carboxy-terminus in the following order: FR1, CDR1, FR2, CDR2, FR3, CDR3, FR4. The variable regions of heavy and light chains contain a binding domain that interacts with an antigen. The constant regions of antibodies may mediate the binding of immunoglobulin to host tissues or factors, including various cells of the immune system (e.g. effector cells) and the first component (C1q) of the classical complement system.

The term “antigen-binding portion” of antibody or “antigen-binding fragment”, as used in the present description, refers to one or more antibody fragments that retain the ability to specifically bind to an antigen. It has been shown that the antigen-binding function of antibody can be performed by fragments of a full-length antibody. As used in the present invention, the term “antigen-binding fragment” means a Fab-fragment, i.e. a monovalent fragment, consisting of VL, VH, CL and CH1 domains, which is linked with the Fc-fragment monomer.

The term “variable” refers to the fact that certain portions of the variable domains greatly differ in sequence among antibodies. The V domain mediates antigen binding and determines specificity of each particular antibody for its particular antigen. However, the variability is not evenly distributed across the 110-amino acid span of the variable domains. Instead, the V regions consist of invariant fragments termed framework regions (FRs) of 15-30 amino acids separated by shorter regions of extreme variability termed “hypervariable regions” or CDRs. The variable domains of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by FRs and, with the hypervariable regions from the other chain, contribute to the formation of the antigen-binding site of antibodies (see Kabat et al., Sequences of Proteins of Immunological Interest. 5 th Ed. Public Health Service, National Institutes of Health, Bethesda, MD. (1991)). The constant domains are not involved directly in binding of antibody to antigen, but exhibit various effector functions, such as participation of antibody in antibody-dependent cellular cytotoxicity (ADCC).

The term “hypervariable region” according to the present description refers to the amino acid residues of antibody which are responsible for antigen binding. The hypervariable region typically comprises amino acid residues from a “complementarity determining region” or “CDR” and/or those residues from a “hypervariable loop”.

“Kabat numbering scheme” or “numbering according to Kabat” refers in the present invention to a system for numbering of amino acid residues that are more variable (i.e. hypervariable) than other amino acid residues in variable regions of heavy and light chains of antibody (Kabat et al. Ann. N.Y. Acad. Sci., 190:382-93 (1971); Kabat et al. Sequences of Proteins of Immunological Interest, Fifth Edition, U.S. Department of Health and Human Services, NIH Publication No. 91-3242 (1991)).

The antibody of the present invention “which binds” a target antigen refers to an antibody that binds the antigen with sufficient affinity such that the antibody can be used as a diagnostic and/or therapeutic agent targeting a protein or cell or tissue expressing the antigen, and slightly cross-reacts with other proteins. According to analytical methods: fluorescence-activated cell sorting (FACS), radioimmunoassay (RIA) or ELISA, in such embodiments, the degree of antibody binding to a non-target protein is less than 10% of antibody binding to a specific target protein. With regard to the binding of antibody to a target molecule, the term “specific binding” or phrases “specifically binds to” or “is specific for” a particular polypeptide or an epitope on a particular target polypeptide means binding that is significantly (measurably) different from a non-specific interaction.

Specific binding may be measured, for example, by determining binding of a molecule as compared to binding of a control molecule. For example, specific binding may be determined by competition with another molecule that is similar to the target, for example, an excess of non-labeled target. In this case, specific binding is indicated if the binding of the labeled target to a probe is competitively inhibited by the excess of unlabeled target. As used in the present description, the term “specific binding” or phrases “specifically binds to” or “specific for” a particular polypeptide or an epitope on a particular target polypeptide may be described by example of a molecule having a KD (affinity constant) for the target of at least about 200 nM, or at least about 150 nM, or at least about 100 nM, or at least about 60 nM, or at least about 50 nM, or at least about 40 nM, or at least about 30 nM, or at least about 20 nM, or at least about 10 nM, or at least about 8 nM, or at least about 6 nM, or at least about 4 nM, or at least about 2 nM, or at least about 1 nM, or greater. In one embodiment, the term “specific binding” refers to binding where a molecule binds to a particular polypeptide or epitope on a particular polypeptide without substantially binding to any other polypeptide or epitope on a polypeptide.

The term “monoclonal antibody” or “mAb” refers to an antibody that is synthesized and isolated as an individual clonal population of cells.

The term “recombinant antibody” refers to an antibody that is expressed in a cell or cell line comprising nucleotide sequence(s) encoding antibodies, wherein said nucleotide sequence(s) is (are) not associated with the cell in nature.

The term “isolated” applied to describe various antibodies according to the present invention refers to an antibody which has been identified and isolated and/or regenerated from a cell or cell culture, in which the antibody is expressed. Impurities (contaminant components) from natural environment are materials which typically interfere with diagnostic or therapeutic uses of the polypeptide, and may include enzymes, hormones, and other proteinaceous or nonproteinaceous solutes. The isolated polypeptide is typically prepared by at least one purification step.

The terms “anti-TRBV9 antibody”, “antibody to TRBV9”, “antibody specifically binding to the TRBV9 family beta-chain” and “antibody against the TRBV9 family beta-chain” are interchangeable within the scope of the present invention and relate to an antibody that specifically binds to the epitope of TRBV9 family beta-chain of human T cell receptor.

The term “pharmaceutical composition” refers to a composition and/or formulation comprising the anti-TRBV9 antibody in a therapeutically effective amount and excipients or auxiliary substances (carriers, diluents, fillers, solvents, etc.), the choice and proportions of which depend on the type and route of administration and dosage.

The term “excipient” or “auxiliary substance” is used herein to describe any ingredient other than the compound(s) of the present invention. These are substances of inorganic or organic nature which are used in the pharmaceutical production/manufacturing in order to give drug products the necessary physicochemical properties.

The term “aqueous composition” as used herein refers to a water-based composition, the water in the composition may be: water, water for injections, physiologic saline (0.9%-1.0% aqueous solution of sodium chloride).

The term “freeze-dried” as used herein refers to a formulation that has been subjected to a process known in the art as freeze-drying, which includes freezing the formulation followed by removal of ice from the frozen contents.

A pharmaceutical composition is “stable” if the active agent retains physical stability and/or chemical stability and/or biological activity thereof during the specified shelf life at a storage temperature, for example, of 2-8° C. Further, the active agent may retain both physical and chemical stability, as well as biological activity. Storage period is adjusted based on the results of stability test in accelerated or natural aging conditions.

The term “long-term storage” or “long term stability” should be understood to mean that a pharmaceutical composition may be stored for three months or more, for six months or more, for one year or more, and the composition may have a minimum stable shelf life of at least two years as well.

The term “buffering agent” refers to an acid or base component (typically a weak acid or weak base) of the buffer or buffer solution. A buffering agent helps to maintain the pH value of a given solution at or near to a pre-determined value, and the buffering agents are generally chosen to complement the pre-determined value. A buffering agent may be a single compound which gives rise to a desired buffering effect, especially when said buffering agent is mixed with (and suitably capable of proton exchange with) an appropriate amount (depending on the pre-determined value desired) of corresponding “acid/base conjugate” thereof.

The term “buffer” or “buffer solution” or “buffer system” refers to an aqueous solution comprising a mixture of an acid (typically a weak acid, such as e.g. acetic acid, citric acid) and a conjugated base thereof (such as e.g. an acetate or citrate salt, e.g. sodium acetate, sodium citrate, as well as hydrates of said salts, e.g. sodium acetate trihydrate) or alternatively a mixture of a base (typically a weak base, e.g. histidine) and a conjugated acid thereof (e.g. histidine hydrochloride or histidine hydrochloride monohydrate or L-histidine hydrochloride (h/c) monohydrate (m/h) or L-histidine h/c m/h or histidine h/c m/h). The pH value of a “buffer solution” changes only slightly upon addition thereto of a small quantity of strong base or strong acid, as well as upon dilution or concentration due to the “buffering effect” imparted by a “buffering agent”.

Buffer solutions may be, for example, acetate, phosphate, citrate, histidine, succinate and other buffer solutions. Generally, the pharmaceutical composition preferably has a pH in the range from 4.0 to 8.0.

“Stabilizer” refers to an excipient or a mixture of two or more excipients that provide the physical and/or chemical stability of the active agent.

The terms “osmotic agent” or “tonicity-regulating agent”, as well as “osmolyte”, as used herein, refer to an excipient that can provide the required osmotic pressure of a liquid antibody solution. In some embodiments, the tonicity-regulating agent may increase the osmotic pressure of a liquid antibody liquid antibody formulation to isotonic pressure such that said liquid antibody formulation is physiologically compatible with the cells of the tissue of a subject's organism. In another embodiment, the tonicity-regulating agent may contribute to increased stability of antibodies. “Isotonic” formulation is a formulation that has an osmotic pressure equivalent to that of human blood. Isotonic formulations typically have an osmotic pressure from about 239 to 376 mOsm/kg.

As used herein, the term “solubilizer” refers to a pharmaceutically acceptable non-ionic surfactant. Both one solubilizer and combinations of solubilizers may be used. Exemplary solubilizers are, without limitation, polysorbate 20 or polysorbate 80, poloxamer 184 or poloxamer 188, or PLURONIC®.

Typically, amino acids are L-amino acids. For example, if histidine and histidine hydrochloride monohydrate are used, it is typically L-histidine and L-histidine hydrochloride monohydrate. For example, if proline is used, it is typically L-proline. Amino acid equivalents, for example, pharmaceutically acceptable proline salts (for example, proline hydrochloride) may also be used.

The term “medicament” or “formulation” refers to a substance (or a mixture of substances in the form of a pharmaceutical composition) in the form of tablets, capsules, solutions, ointments and other ready forms intended for restoration, improvement or modification of physiological functions in humans and animals, and for treatment and prophylaxis of diseases, for diagnostics, anesthesia, contraception, cosmetology and others.

The term “use” applies to the possibility to use the pharmaceutical composition of anti-TRBV9 antibody according to the present invention to treat, relief the course of the disease or disorders, expedite the remission, reduce the recurrence rate for the diseases or disorders.

The term “disease or disorder mediated by T-lymphocytes bearing a TRBV9 segment within the T cell receptor” refers to any disease or disorder that is either directly, or indirectly associated with T-lymphocytes bearing a TRBV9 segment within the T cell receptor, including etiology, development, progression, persistence or pathology of a disease or disorder.

“Treat”, “treatment” and “therapy” refer to a method of alleviating or abrogating a biological disorder and/or at least one of attendant symptoms thereof. As used herein, to “alleviate” a disease, disorder or condition means reducing the severity and/or occurrence frequency of the symptoms of the disease, disorder, or condition. Further, references herein to “treatment” include references to curative, palliative and prophylactic treatment.

The term “parenteral administration” refers to administration regimens, typically performed by injection (infusion), and includes, in particular intravenous, intramuscular, intraarterial, intratracheal, intracapsular, intraorbital, intracardiac, intradermal, intraperitoneal, transtracheal, subcutaneous, intraarticular, subcapsular, subarchnoid, intraspinal, epidural and intrasternal injection or infusion.

### ABBREVIATIONS

anti-TRBV9 (anti-TRBV9)—monoclonal antibody to TRBV9
IC—incoming control
FT—freeze-thaw
k_{D}—diffusion interaction parameter
SH—shake
Tag—aggregation temperature
Tonset—melting onset temperature
Tm—melting point
T—temperature
TS—thermal stress
C—protein concentration
Osm—osmolality
TS50 96H—thermal stress at 50° C. for 96 hours
Δ TS50 96H—change in the quality parameter following thermal stress at 50° C. for 120 hours
TS50 120H—thermal stress at 50° C. for 96 hours
Δ TS50 120H—change in the quality parameter following thermal stress at 50° C. for 120 hours
Acid 3.0 24H acid hydrolysis to pH 3.0 and ageing for 1 or 24 hours
Δ Acid 3.0 24H—change in the quality parameter following stress by acid hydrolysis to pH 3.0 and ageing for 1 or 24 hours
Basic 9.0 1H—basic hydrolysis to pH 9.0 and ageing for 1 hour
Δ Basic 9.0 1H—change in the quality parameter following stress by alkaline hydrolysis to pH 9.0 and ageing for 1 hour
SH800 96H—shaking for 96 hours at 800 rpm
Δ SH800 96H—change in the quality parameter following shaking for 96 hours at 800 rpm
SH800 120H—shaking for 120 hours at 800 rpm
Δ SH800 120H—change in the quality parameter following shaking for 120 hours at 800 rpm
fr-th 3 cycle—three freeze-thaw cycles
Δ fr-th 3 cycle—change in the quality parameter following three freeze-thaw cycles
FT 5C—five freeze-thaw cycles
Δ FT 5C—change in the quality parameter following five freeze-thaw cycles
IE HPLC—ion exchange high-performance liquid chromatography
SW—software
SE HPLC—size-exclusive high-performance liquid
n/a—not determined
abs—absolute change in the quality parameter
C con—concentration following concentrating
DSF—differential scanning fluorimetry
DLS—dynamic light scattering
ABP—acid-base profile
Oxid 0.1%—oxidation by 0.1% hydrogen peroxide solution
Δ Oxid 0.1%—change in the quality parameter following oxidation by 0.1% hydrogen peroxide solution
Surfactant—surface-active agent
AS37—accelerated storage at 37° C.
2W—2 weeks
4W—4 weeks
Δ AS37—change in the quality parameter following accelerated storage at 37° C.
Max—maximum value
Min—minimum value
pH—hydrogen index
Cfin./Cin.—ratio of concentrations before and following concentrating
N—viscosity
CE—capillary electrophoresis
Red.—reducing conditions
Non-red.—non-reducing conditions

The present invention discloses stable pharmaceutical compositions of anti-TRBV9 antibody, which may be used as a medicinal product to treat diseases or disorders mediated by T-lymphocytes bearing a TRBV9 segment within the T cell receptor.

In one aspect, the present invention relates to a pharmaceutical composition that comprises at least one anti-TRBV9 antibody in a therapeutically effective amount in combination with one or more pharmaceutically acceptable excipients. In one aspect, the present invention relates to a pharmaceutical composition that comprises anti-TRBV9 antibody in combination with one or more pharmaceutically acceptable excipients.

During formulation selection, we took into account the purpose, route of administration and tolerability of the drug product (for example, reduction of discomfort during administration), as well as the stability and preservation of activity of protein molecule within the formulation.

In one aspect, the present invention relates to a pharmaceutical composition comprising:
(i) anti-TRBV9 antibody;
(ii) histidine buffer or acetate buffer;
(iii) water for injection.

The anti-TRBV9 antibody may be an antibody that specifically binds to the TRBV9 family beta chain. The anti-TRBV9 antibody may be a full-length antibody or antigen-binding fragment thereof that specifically binds to the TRBV9 family beta chain. The anti-TRBV9 antibody may be of different specificities (e.g. monospecific, bispecific antibody), different valencies (e.g. monovalent, bivalent, trivalent antibody), different formats (e.g. classical antibody, scFv, scFv-Fc, Minibody), different origins (e.g. murine, human, camel, chimeric antibody).

In some embodiments of the invention, the anti-TRBV9 antibody relates to an isolated monoclonal antibody.

In some embodiments of the invention, the anti-TRBV9 antibody relates to a monospecific antibody.

In some embodiments of the invention, the anti-TRBV9 antibody is a recombinant antibody.

In some embodiments of the invention, the anti-TRBV9 antibody comprises:

1) a heavy chain variable domain comprising:
(a) HCDR1 having the amino acid sequence of SEQ ID NO: 1,
(b) HCDR2 having the amino acid sequence of SEQ ID NO: 2, and
(c) HCDR3 having an amino acid sequence selected from the group consisting of SEQ ID NO: 3, SEQ ID NO: 4, SEQ ID NO: 5 or SEQ ID NO: 6;

2) a light chain variable domain comprising:
(a) LCDR1 having the amino acid sequence of SEQ ID NO: 7,
(b) LCDR2 having the amino acid sequence of SEQ ID NO: 8, and
(c) LCDR3 having the amino acid sequence of SEQ ID NO: 9.

Antibodies according to the invention may be of any class (e.g. IgA, IgD, IgE, IgG, and IgM), or subclass (isotype) (e.g. IgG1, IgG2, IgG3, IgG4, IgA1 and IgA2).

In some embodiments of the invention, the anti-TRBV9 antibody is a full-length IgG antibody.

In some embodiments of the invention, the anti-TRBV9 antibody is of human IgG1, IgG2, IgG3 or IgG4 isotype.

In some embodiments of the invention, the anti-TRBV9 antibody comprises a heavy chain variable domain having the amino acid sequence of SEQ ID No: 14 and a light chain variable domain having the amino acid sequence of SEQ ID No: 17.

In some embodiments of the invention, the anti-TRBV9 antibody comprises a heavy chain having the amino acid sequence of SEQ ID No: 22 and a light chain having the amino acid sequence of SEQ ID No: 25 (candidate 42 or antibody 42).

In some embodiments of the invention, the anti-TRBV9 antibody comprises a heavy chain variable domain having the amino acid sequence of SEQ ID No: 15 and a light chain variable domain having the amino acid sequence of SEQ ID No: 17.

In some embodiments of the invention, the anti-TRBV9 antibody comprises a heavy chain having the amino acid sequence of SEQ ID No: 23 and a light chain having the amino acid sequence of SEQ ID No: 25 (candidate 43 or antibody 43).

The concentration of anti-TRBV9 antibody contained in the pharmaceutical compositions of the present invention may vary depending on the desired properties of the compositions, as well as on the particular conditions, methods and purposes of use of the pharmaceutical compositions.

In some embodiments of the invention, the anti-TRBV9 antibody is present at a concentration of 0.5-300.0 mg/ml. In some embodiments of the invention, the anti-TRBV9 antibody is present at a concentration of 0.5-280.0 mg/ml. In some embodiments of the invention, the anti-TRBV9 antibody is present at a concentration of 0.5-250.0 mg/ml.

In some embodiments of the invention, the anti-TRBV9 antibody is present at a concentration of 0.5-225.0 mg/ml.

In some embodiments of the invention, the anti-TRBV9 antibody is present at a concentration of 1.5-190.0 mg/ml, or 200.0-225.0 mg/ml, or 240.0-300.0 mg/ml; or 1.5-125.0 mg/ml, or 150.0-225.0 mg/ml, or 240.0-300.0 mg/ml; or 1.5-125.0 mg/ml, or 180.0-225.0 mg/ml, or 240.0-300.0 mg/ml; or 1.5-95 mg/ml, or 100-125.0 mg/ml, or 150.0-225.0 mg/ml, or 240.0-300.0 mg/ml; or 1.5-95 mg/ml, or 100-125.0 mg/ml, or 180.0-225.0 mg/ml, or 240.0-300.0 mg/ml; or 1.5-85 mg/ml, or 90.0-125.0 mg/ml, or 150.0-225.0 mg/ml, or 240.0-300.0 mg/ml; or 1.5-95 mg/ml, or 100-125.0 mg/ml, or 180.0-225.0 mg/ml, or 240.0-300.0 mg/ml; or 1.5-75 mg/ml, or 80.0-125.0 mg/ml, or 150.0-225.0 mg/ml, or 240.0-300.0 mg/ml; or 1.5-75 mg/ml, or 80.0-125.0 mg/ml, or 180.0-225.0 mg/ml, or 240.0-300.0 mg/ml; or 1.5-50.0 mg/ml, or 60.0-125.0 mg/ml, or 150.0-225.0 mg/ml, or 240.0-300.0 mg/ml; or 1.5-50.0 mg/ml, or 60.0-125.0 mg/ml, or 180.0-225.0 mg/ml, or 240.0-300.0 mg/ml; or 1.5-30.0 mg/ml, or 40.0-125.0 mg/ml, or 150.0-225.0 mg/ml, or 240.0-300.0 mg/ml; or 1.5-30.0 mg/ml, or 40.0-125.0 mg/ml, or 180.0-225.0 mg/ml, or 240.0-300.0 mg/ml.

In some embodiments of the invention, the anti-TRBV9 antibody is present at a concentration of 1.5-50.0 mg/ml, or 60.0-150.0 mg/ml, or 180.0-225.0 mg/ml, or 240.0-300.0 mg/ml.

In some embodiments of the invention, the anti-TRBV9 antibody is present at a concentration of 1.5-35.0 mg/ml, or 40.0-60.0 mg/ml, or 70.0-125.0 mg/ml, or 180.0-225.0 mg/ml, or 240.0-300.0 mg/ml.

In some embodiments of the invention, the anti-TRBV9 antibody is present at a concentration of 4.0-6.0 mg/ml, or 8.0-12.0 mg/ml, or 23.0-32.0 mg/ml, or 40.0-60.0 mg/ml, or 70.0-105.0 mg/ml, or 180.0-225.0 mg/ml, or 240.0-300.0 mg/ml.

In some embodiments of the invention, the anti-TRBV9 antibody is present at a concentration of 4.0-6.0 mg/ml, or 8.0-12.0 mg/ml, or 23.0-32.0 mg/ml, or 50.0-105.0 mg/ml, or 180.0-225.0 mg/ml, or 240.0-300.0 mg/ml.

In some embodiments of the invention, the anti-TRBV9 antibody is present at a concentration of 4.0-6.0 mg/ml, or 8.0-12.0 mg/ml, or 23.0-32.0 mg/ml, or 70.0-105.0 mg/ml, or 180.0-225.0 mg/ml, or 240.0-300.0 mg/ml.

In some embodiments of the invention, the anti-TRBV9 antibody is present at a concentration of 1.5 mg/ml, or 5.0 mg/ml, or 10.0 mg/ml, or 25.0 mg/ml, or 30.0 mg/ml, or 40.0 mg/ml, or 50.0 mg/ml, or 60.0 mg/ml, or 70.0 mg/ml, or 73.0 mg/ml, or 80.0 mg/ml, or 85.0 mg/ml, or 90.0 mg/ml, or 91.4 mg/ml, or 91.8 mg/ml, or 100.0 mg/ml, or 103.0 mg/ml, or 125.0 mg/ml, or 186.0 mg/ml, or 212.0 mg/ml.

In some embodiments of the invention, the pharmaceutical composition comprises:
(i) anti-TRBV9 antibody;
(ii) histidine buffer;
(iii) water for injection.

In some embodiments of the invention, the histidine buffer is a mixture of histidine and histidine hydrochloride monohydrate.

In some embodiments of the invention, histidine is present at a concentration of 0.4-14.11 mg/ml.

In some embodiments of the invention, histidine is present at a concentration of 0.4-11.0 mg/ml.

In some embodiments of the invention, histidine is present at a concentration of 0.4-10.0 mg/ml.

In some embodiments of the invention, histidine is present at a concentration of 0.4-8.0 mg/ml.

In some embodiments of the invention, histidine is present at a concentration of 0.4-5.0 mg/ml.

In some embodiments of the invention, histidine is present at a concentration of 0.4-3.0 mg/ml.

In some embodiments of the invention, histidine is present at a concentration of 0.4-1.5 mg/ml.

In some embodiments of the invention, histidine is present at a concentration of 0.4-1.0 mg/ml.

In some embodiments of the invention, histidine is present at a concentration of 0.4-0.8 mg/ml.

In some embodiments of the invention, histidine is present at a concentration of 0.45-0.8 mg/ml.

In some embodiments of the invention, histidine is present at a concentration of 0.5-0.8 mg/ml.

In some embodiments of the invention, histidine is present at a concentration of 0.45-0.6 mg/ml, or 0.65-0.8 mg/ml.

In some embodiments of the invention, histidine is present at a concentration of 0.5-0.6 mg/ml, or 0.65-0.8 mg/ml.

In some embodiments of the invention, histidine is present at a concentration of 0.517 mg/ml, or 0.580 mg/ml, or 0.689 mg/ml, or 0.746 mg/ml.

In some embodiments of the invention, histidine hydrochloride monohydrate is present at a concentration of 0.05-19.06 mg/ml.

In some embodiments of the invention, histidine hydrochloride monohydrate is present at a concentration of 0.05-15.0 mg/ml.

In some embodiments of the invention, histidine hydrochloride monohydrate is present at a concentration of 0.05 to 12.0 mg/ml.

In some embodiments of the invention, histidine hydrochloride monohydrate is present at a concentration of 0.05 to 10.0 mg/ml.

In some embodiments of the invention, histidine hydrochloride monohydrate is present at a concentration of 0.05 to 8.0 mg/ml.

In some embodiments of the invention, histidine hydrochloride monohydrate is present at a concentration of 0.05 to 6.0 mg/ml.

In some embodiments of the invention, histidine hydrochloride monohydrate is present at a concentration of 0.05 to 5.0 mg/ml.

In some embodiments of the invention, histidine hydrochloride monohydrate is present at a concentration of 0.05 to 4.5 mg/ml.

In some embodiments of the invention, histidine hydrochloride monohydrate is present at a concentration of 0.08 to 4.2 mg/ml.

In some embodiments of the invention, histidine hydrochloride monohydrate is present at a concentration of 0.08 to 4.0 mg/ml.

In some embodiments of the invention, histidine hydrochloride monohydrate is present at a concentration of 0.1 to 4.2 mg/ml.

In some embodiments of the invention, histidine hydrochloride monohydrate is present at a concentration of 0.1 to 4.0 mg/ml.

In some embodiments of the invention, histidine hydrochloride monohydrate is present at a concentration of 0.1 to 3.5 mg/ml.

In some embodiments of the invention, histidine hydrochloride monohydrate is present at a concentration of 0.08 to 1.0 mg/ml or 2.0 to 4.2 mg/ml.

In some embodiments of the invention, histidine hydrochloride monohydrate is present at a concentration of 0.08 to 0.15 mg/ml, or 0.2 to 0.4 mg/ml, or 2.2 to 4.2 mg/ml.

In some embodiments of the invention, histidine hydrochloride monohydrate is present at a concentration of 0.117 mg/ml, or 0.270 mg/ml, or 0.350 mg/ml, or 3.185 mg/ml.

In some embodiments of the invention,
the histidine buffer is a mixture of:

| | |
|---|---|
| histidine | 0.4-1.0 mg/ml and |
| histidine hydrochloride monohydrate | 0.08-4.2 mg/ml. |

In some embodiments of the invention,
the histidine buffer is a mixture of:

| | |
|---|---|
| histidine | 0.517 mg/ml and |
| histidine hydrochloride monohydrate | 0.350 mg/ml. |

In some embodiments of the invention,
the histidine buffer is a mixture of:

| | |
|---|---|
| histidine | 0.746 mg/ml and |
| histidine hydrochloride monohydrate | 3.185 mg/ml. |

In some embodiments of the invention,
the histidine buffer is a mixture of:

| | |
|---|---|
| histidine | 0.580 mg/ml and |
| histidine hydrochloride monohydrate | 0.270 mg/ml. |

In some embodiments of the invention,
the histidine buffer is a mixture of:

| | |
|---|---|
| histidine | 0.689 mg/ml and |
| histidine hydrochloride monohydrate | 0.117 mg/ml. |

In some embodiments of the invention,
the pharmaceutical composition comprises:

| | |
|---|---|
| (i) anti-TRBV9 antibody; | 0.517 mg/ml and |
| (ii) histidine buffer being a mixture of histidine | 0.350 mg/ml; |
| histidine hydrochloride monohydrate | to 1 ml. |
| (iii) water for injection | |

In some embodiments of the invention,
the pharmaceutical composition comprises:

| | |
|---|---|
| (i) anti-TRBV9 antibody; | 0.746 mg/ml and |
| (ii) histidine buffer being a mixture of histidine | 3.185 mg/ml; |
| histidine hydrochloride monohydrate | to 1 ml. |
| (iii) water for injection | |

In some embodiments of the invention,
the pharmaceutical composition comprises:

| | |
|---|---|
| (i) anti-TRBV9 antibody; | 0.580 mg/ml and |
| (ii) histidine buffer being a mixture of histidine | 0.270 mg/ml; |
| histidine hydrochloride monohydrate | to 1 ml. |
| (iii) water for injection | |

In some embodiments of the invention,
the pharmaceutical composition comprises:

| | |
|---|---|
| (i) anti-TRBV9 antibody; | 0.689 mg/ml and |
| (ii) histidine buffer being a mixture of histidine | 0.117 mg/ml; |
| histidine hydrochloride monohydrate | to 1 ml. |
| (iii) water for injection | |

In some embodiments of the invention, the anti-TRBV9 antibody is present at a concentration of 0.5-300.0 mg/ml, or 1.5-225.0 mg/ml, or 5.0-125.0 mg/ml, or 5.0-100.0 mg/ml, 5.0-50.0 mg/ml, or 5.0-30.0 mg/ml.

In some embodiments of the invention, the anti-TRBV9 antibody is present at a concentration of 1.5 mg/ml, or 5.0 mg/ml, or 10.0 mg/ml, or 25.0 mg/ml, or 30.0 mg/ml, or 40.0 mg/ml, or 50.0 mg/ml, or 60.0 mg/ml, or 70.0 mg/ml, or 73.0 mg/ml, or 80.0 mg/ml, or 85.0 mg/ml, or 90.0 mg/ml, or 91.4 mg/ml, or 91.8 mg/ml, or 100.0 mg/ml, or 103.0 mg/ml, or 125.0 mg/ml, or 186.0 mg/ml, or 212.0 mg/ml, 225.0 mg/ml, 300.0 mg/ml.

In some embodiments of the invention,
the pharmaceutical composition comprises:

| | |
|---|---|
| (i) anti-TRBV9 antibody | 0.5-300.0 mg/ml; |
| (ii) histidine buffer being a mixture of histidine | 0.4-1.0 mg/ml and |
| histidine hydrochloride monohydrate | 0.08-4.2 mg/ml; |
| (iii) water for injection | to 1 ml. |

In some embodiments of the invention,
the pharmaceutical composition comprises:

| | |
|---|---|
| (i) anti-TRBV9 antibody | 5-125.0 mg/ml; |
| (ii) histidine buffer being a mixture of histidine | 0.4-1.0 mg/ml and |
| histidine hydrochloride monohydrate | 0.08-4.2 mg/ml; |
| (iii) water for injection | to 1 ml. |

In some embodiments of the invention,
the pharmaceutical composition comprises:

| | |
|---|---|
| (i) anti-TRBV9 antibody | 5-100.0 mg/ml; |
| (ii) histidine buffer being a mixture of histidine | 0.4-1.0 mg/ml and |
| histidine hydrochloride monohydrate | 0.08-4.2 mg/ml; |
| (iii) water for injection | to 1 ml. |

In some embodiments of the invention,
the pharmaceutical composition comprises:

| | |
|---|---|
| (i) anti-TRBV9 antibody | 25 mg/ml; |
| (ii) histidine buffer being a mixture of histidine | 0.4-1.0 mg/ml and |
| histidine hydrochloride monohydrate | 0.08-4.2 mg/ml; |
| (iii) water for injection | to 1 ml. |

In some embodiments of the invention,
the pharmaceutical composition comprises:

| | |
|---|---|
| (i) anti-TRBV9 antibody | 25 mg/ml; |
| (ii) histidine buffer being a mixture of histidine | 0.517 mg/ml and |
| histidine hydrochloride monohydrate | 0.350 mg/ml; |
| (iii) water for injection | to 1 ml. |

In some embodiments of the invention,
the pharmaceutical composition comprises:

| | |
|---|---|
| (i) anti-TRBV9 antibody; | 0.5-300.0 mg/ml; |
| (ii) histidine buffer being a mixture of histidine | 0.517 mg/ml and |
| histidine hydrochloride monohydrate | 0.350 mg/ml; |
| (iii) water for injection | to 1 ml. |

In some embodiments of the invention,
the pharmaceutical composition comprises:

| | |
|---|---|
| (i) anti-TRBV9 antibody; | 5-125.0 mg/ml; |
| (ii) histidine buffer being a mixture of histidine | 0.517 mg/ml and |
| histidine hydrochloride monohydrate | 0.350 mg/ml; |
| (iii) water for injection | to 1 ml. |

In some embodiments of the invention,
the pharmaceutical composition comprises:

| | |
|---|---|
| (i) anti-TRBV9 antibody; | 5-100.0 mg/ml; |
| (ii) histidine buffer being a mixture of histidine | 0.517 mg/ml and |
| histidine hydrochloride monohydrate | 0.350 mg/ml; |
| (iii) water for injection | to 1 ml. |

In some embodiments of the invention, the pharmaceutical composition comprises:
(i) anti-TRBV9 antibody;
(ii) acetate buffer;
(iii) water for injection.

In some embodiments of the invention, the acetate buffer is a mixture of sodium acetate and acetic acid.

In some embodiments of the invention, sodium acetate is present at a concentration of 0.014-12.88 mg/ml.

In some embodiments of the invention, sodium acetate is present at a concentration of 0.014-8.0 mg/ml.

In some embodiments of the invention, sodium acetate is present at a concentration of 0.5-3.0 mg/ml.

In some embodiments of the invention, sodium acetate is present at a concentration of 0.5-2.5 mg/ml.

In some embodiments of the invention, sodium acetate is present at a concentration of 0.5-0.8 mg/ml, or 1.6-3.0 mg/ml.

In some embodiments of the invention, sodium acetate is present at a concentration of 0.644 mg/ml, or 2.311 mg/ml.

In some embodiments of the invention, sodium acetate is sodium acetate trihydrate.

In some embodiments of the invention, acetic acid is added to pH 3.5-6.1.

In some embodiments of the invention, acetic acid is added to pH 5.4-6.1.

In some embodiments of the invention, acetic acid is added to pH 5.4-5.6, or to pH 5.9-6.1.

In some embodiments of the invention, acetic acid is added to pH 5.5 or to pH 6.0.

In some embodiments of the invention, acetic acid is glacial acetic acid.

In some embodiments of the invention, the acetate buffer is a
mixture of:

| | |
|---|---|
| sodium acetate | 0.5-3.0 mg/ml and |
| acetic acid | to pH 5.4-6.1. |

In some embodiments of the invention, the acetate buffer is a
mixture of:

| | |
|---|---|
| sodium acetate | 0.5-2.5 mg/ml and |
| acetic acid | to pH 5.4-6.1. |

In some embodiments of the invention, the acetate buffer is a
mixture of:

| | |
|---|---|
| sodium acetate | 0.644 mg/ml and |
| acetic acid | to pH 6.0. |

In some embodiments of the invention, the acetate buffer is a
mixture of:

| | |
|---|---|
| sodium acetate | 2.311 mg/ml and |
| acetic acid | to pH 5.5. |

In some embodiments of the invention, the pharmaceutical
composition comprises:

| | |
|---|---|
| (i) anti-TRBV9 antibody; | |
| (ii) acetate buffer being a mixture of | 0.644 mg/ml and |
| sodium acetate | to pH 6.0. |
| acetic acid | |
| (iii) water for injection | to 1 ml. |

In some embodiments of the invention, the pharmaceutical
composition comprises:

| | |
|---|---|
| (i) anti-TRBV9 antibody; | |
| (ii) acetate buffer being a mixture of | 2.311 mg/ml and |
| sodium acetate | to pH 5.5. |
| acetic acid | |
| (iii) water for injection | to 1 ml. |

In some embodiments of the invention, the anti-TRBV9 antibody is present at a concentration of 0.5-300.0 mg/ml, or 1.5-225.0 mg/ml, or 5.0-125.0 mg/ml, or 5.0-100.0 mg/ml, 5.0-50.0 mg/ml, or 5.0-30.0 mg/ml.

In some embodiments of the invention, the anti-TRBV9 antibody is present at a concentration of 1.5 mg/ml, or 5.0 mg/ml, or 10.0 mg/ml, or 25.0 mg/ml, or 30.0 mg/ml, or 40.0 mg/ml, or 50.0 mg/ml, or 60.0 mg/ml, or 70.0 mg/ml, or 73.0 mg/ml, or 80.0 mg/ml, or 85.0 mg/ml, or 90.0 mg/ml, or 91.4 mg/ml, or 91.8 mg/ml, or 100.0 mg/ml, or 103.0 mg/ml, or 125.0 mg/ml, or 186.0 mg/ml, or 212.0 mg/ml, 225.0 mg/ml, 300.0 mg/ml.

In some embodiments of the invention, the pharmaceutical
composition comprises:

| | |
|---|---|
| (i) anti-TRBV9 antibody | 0.5-300.0 mg/ml; |
| (ii) acetate buffer being a mixture of | 0.5-3.0 mg/ml and |
| sodium acetate | to pH 5.4-6.1; |
| acetic acid | |
| (iii) water for injection | to 1 ml. |

In some embodiments of the invention, the pharmaceutical
composition comprises:

| | |
|---|---|
| (i) anti-TRBV9 antibody | 0.5-125.0 mg/ml; |
| (ii) acetate buffer being a mixture of | 0.5-3.0 mg/ml and |
| sodium acetate | to pH 5.4-6.1; |
| acetic acid | |
| (iii) water for injection | to 1 ml. |

In some embodiments of the invention, the pharmaceutical
composition comprises:

| | |
|---|---|
| (iv) anti-TRBV9 antibody | 0.5-100.0 mg/ml; |
| (v) acetate buffer being a mixture of | 0.5-3.0 mg/ml and |
| sodium acetate | to pH 5.4-6.1; |
| acetic acid | |
| (vi) water for injection | to 1 ml. |

In some embodiments of the invention, the pharmaceutical
composition comprises:

| | |
|---|---|
| (i) anti-TRBV9 antibody | 25.0 mg/ml; |
| (ii) acetate buffer being a mixture of | 0.5-3.0 mg/ml and |
| sodium acetate | to pH 5.4-6.1; |
| acetic acid | |
| (iii) water for injection | to 1 ml. |

In some embodiments of the invention, the pharmaceutical
composition comprises:

| | |
|---|---|
| (i) anti-TRBV9 antibody | 0.5-300.0 mg/ml; |
| (ii) acetate buffer being a mixture of | 0.644 mg/ml and |
| sodium acetate | to pH 6.0; |
| acetic acid | |
| (iii) water for injection | to 1 ml. |

In some embodiments of the invention, the pharmaceutical
composition comprises:

| | |
|---|---|
| (i) anti-TRBV9 antibody | 0.5-300.0 mg/ml; |
| (ii) acetate buffer being a mixture of | 2.311 mg/ml and |
| sodium acetate | to pH 5.5; |
| acetic acid | |
| (iii) water for injection | to 1 ml. |

In some embodiments of the invention, the pharmaceutical
composition comprises:

| | |
|---|---|
| (i) anti-TRBV9 antibody | 5-125.0 mg/ml; |
| (ii) acetate buffer being a mixture of | 0.644 mg/ml and |
| sodium acetate | to pH 6.0; |
| acetic acid | |
| (iii) water for injection | to 1 ml. |

In some embodiments of the invention, the pharmaceutical
composition comprises:

| | |
|---|---|
| (iv) anti-TRBV9 antibody | 5-100.0 mg/ml; |
| (v) acetate buffer being a mixture of | 0.644 mg/ml and |
| sodium acetate | to pH 6.0; |
| acetic acid | |
| (vi) water for injection | to 1 ml. |

In some embodiments of the invention, the pharmaceutical
composition comprises:

| | |
|---|---|
| (i) anti-TRBV9 antibody | 5-125.0 mg/ml; |
| (ii) acetate buffer being a mixture of | 2.311 mg/ml and |
| sodium acetate | to pH 5.5; |
| acetic acid | |
| (iii) water for injection | to 1 ml. |

In some embodiments of the invention, the pharmaceutical
composition comprises:

| | |
|---|---|
| (iv) anti-TRBV9 antibody | 5-100.0 mg/ml; |
| (v) acetate buffer being a mixture of | 2.311 mg/ml and |
| sodium acetate | to pH 5.5; |
| acetic acid | |
| (vi) water for injection | to 1 ml. |

In some embodiments of the invention, the pharmaceutical
composition comprises:

| | |
|---|---|
| (i) anti-TRBV9 antibody | 25.0 mg/ml; |
| (ii) acetate buffer being a mixture of | 0.644 mg/ml and |
| sodium acetate | to pH 6.0; |
| acetic acid | |
| (iii) water for injection | to 1 ml. |

In some embodiments of the invention, the pharmaceutical
composition comprises:

| | |
|---|---|
| (i) anti-TRBV9 antibody | 25.0 mg/ml; |
| (ii) acetate buffer being a mixture of | 2.311 mg/ml and |
| sodium acetate | to pH 5.5; |
| acetic acid | |
| (iii) water for injection | to 1 ml. |

In some embodiments of the invention, the pharmaceutical
composition further comprises one or more osmotic agents.
In some embodiments of the invention, the pharmaceutical
composition comprises:

(i) anti-TRBV9 antibody;
(ii) histidine buffer or acetate buffer;
(iii) osmotic agent;
(iv) water for injection.

In some embodiments of the invention, the pharmaceutical
composition comprises:

| | |
|---|---|
| (i) anti-TRBV9 antibody; | 0.5-300.0 mg/ml; |
| (ii) histidine buffer or acetate buffer; | |
| (iii) osmotic agent; | |
| (iv) water for injection | to 1 ml. |

The osmotic agent may be present in an enantiomeric (e.g. L- or D-enantiomer) or racemic form; in the form of isomers such as alpha or beta, including alpha, alpha; or beta, beta; or alpha, beta; or beta, alpha; in the form of a free acid or free base; in the form of a salt; in a hydrated form (e.g. monohydrate or dihydrate), or in an anhydrous form. Exemplary osmotic agents are, but not limited to, sugars (trehalose, trehalose dihydrate, sucrose, glucose), polyols (mannitol, sorbitol), amino acids (proline or L-proline, arginine or L-arginine, glycine or L-glycine), or salts (sodium chloride, potassium chloride, magnesium chloride).

In some embodiments of the invention, the osmotic agent is present at a concentration of 0.001-200.0 mg/ml.

In some embodiments of the invention, the osmotic agent is present at a concentration of 0.001-180.0 mg/ml.

In some embodiments of the invention, the osmotic agent is present at a concentration of 0.001-150.0 mg/ml.

In some embodiments of the invention, the osmotic agent is present at a concentration of 0.001-130.0 mg/ml.

In some embodiments of the invention, the osmotic agent is present at a concentration of 6.0-130.0 mg/ml.

In some embodiments of the invention, the osmotic agent is proline, sorbitol, trehalose or sodium chloride.

In some embodiments of the invention, proline is present at a concentration of 0.001-60.0 mg/ml.

In some embodiments of the invention, proline is present at a concentration of 14.0-32.0 mg/ml.

In some embodiments of the invention, proline is present at a concentration of 17.0-32.0 mg/ml.

In some embodiments of the invention, proline is present at a concentration of 17.0-23.0 mg/ml or 25.0-29.0 mg/ml.

In some embodiments of the invention, proline is present at a concentration of 19.0 mg/ml, or 21.0 mg/ml, or 27.0 mg/ml.

In some embodiments of the invention, sorbitol is present at a concentration of 0.001-100.0 mg/ml.

In some embodiments of the invention, sorbitol is present at a concentration of 20.0-80.0 mg/ml.

In some embodiments of the invention, sorbitol is present at a concentration of 35.0-65.0 mg/ml.

In some embodiments of the invention, sorbitol is present at a concentration of 40.0-60.0 mg/ml.

In some embodiments of the invention, sorbitol is present at a concentration of 45.0-55.0 mg/ml.

In some embodiments of the invention, sorbitol is present at a concentration of 50.0 mg/ml.

In some embodiments of the invention, trehalose is present at a concentration of 0.001-200.0 mg/ml.

In some embodiments of the invention, trehalose is present at a concentration of 0.001-180.0 mg/ml.

In some embodiments of the invention, trehalose is present at a concentration of 40.0-160.0 mg/ml.

In some embodiments of the invention, trehalose is present at a concentration of 60.0-140.0 mg/ml.

In some embodiments of the invention, trehalose is present at a concentration of 70.0-130.0 mg/ml.

In some embodiments of the invention, trehalose is present at a concentration of 80.0-120.0 mg/ml.

In some embodiments of the invention, trehalose is present at a concentration of 90.0-110.0 mg/ml.

In some embodiments of the invention, trehalose is present at a concentration of 100.0 mg/ml.

In some embodiments of the invention, sodium chloride is present at a concentration of 0.001-18.0 mg/ml.

In some embodiments of the invention, sodium chloride is present at a concentration of 3.0-16.0 mg/ml.

In some embodiments of the invention, sodium chloride is present at a concentration of 5.0-14.0 mg/ml.

In some embodiments of the invention, sodium chloride is present at a concentration of 7.0-12.0 mg/ml.

In some embodiments of the invention, sodium chloride is present at a concentration of 7.0-11.0 mg/ml.

In some embodiments of the invention, sodium chloride is present at a concentration of 7.5-11.5 mg/ml.

In some embodiments of the invention, sodium chloride is present at a concentration of 7.5-10.5 mg/ml.

In some embodiments of the invention, sodium chloride is present at a concentration of 8.0-10.0 mg/ml.

In some embodiments of the invention, sodium chloride is present at a concentration of 8.5-9.5 mg/ml.

In some embodiments of the invention, sodium chloride is present at a concentration of 9.0 mg/ml.

In some embodiments of the invention, the pharmaceutical composition further comprises one or more stabilizers.

Stabilizers may be amino acids, for example, but not limited to, arginine, histidine, glycine, lysine, glutamine, proline; surfactants, for example, but not limited to, polysorbate 20 (trade name: Tween 20), polysorbate 80 (trade name: Tween 80), polyethylene-polypropylene glycol and copolymers thereof (trade names: Poloxamer, Pluronic, sodium dodecyl sulfate (SDS); antioxidants, for example, but not limited to, methionine, acetylcysteine, ascorbic acid, monothioglycerol, sulfurous acid salts, etc.; chelating agents, for example, but not limited to, ethylenediaminetetraacetic acid (EDTA), diethylenetriaminepentaacetic acid (DTPA), sodium citrate, etc.

In some embodiments of the invention, the stabilizer is present at a concentration of 0.001-100.0 mg/ml.

In some embodiments of the invention, the stabilizer is present at a concentration of 0.001-50.0 mg/ml.

In some embodiments of the invention, the stabilizer is present at a concentration of 0.001-30.0 mg/ml.

In some embodiments of the invention, the stabilizer is present at a concentration of 0.35-9.5 mg/ml.

In some embodiments of the invention, the stabilizer is an amino acid or surfactant.

In some embodiments of the invention, the amino acid is present at a concentration of 0.001-100.0 mg/ml.

In some embodiments of the invention, the amino acid is present at a concentration of 0.001-50.0 mg/ml.

In some embodiments of the invention, the amino acid is present at a concentration of 0.001-30.0 mg/ml.

In some embodiments of the invention, the amino acid is present at a concentration of 0.35-9.5 mg/ml.

In some embodiments of the invention, the amino acid is present at a concentration of 1.1-9.5 mg/ml.

In some embodiments of the invention, the amino acid is glycine or methionine.

In some embodiments of the invention, glycine is present at a concentration of 0.001-100.0 mg/ml.

In some embodiments of the invention, glycine is present at a concentration of 0.001-80.0 mg/ml.

In some embodiments of the invention, glycine is present at a concentration of 0.001-60.0 mg/ml.

In some embodiments of the invention, glycine is present at a concentration of 0.001-40.0 mg/ml.

In some embodiments of the invention, glycine is present at a concentration of 0.001-20.0 mg/ml.

In some embodiments of the invention, glycine is present at a concentration of 0.001-15.0 mg/ml.

In some embodiments of the invention, glycine is present at a concentration of 3.0-12.0 mg/ml.

In some embodiments of the invention, glycine is present at a concentration of 5.5-9.5 mg/ml.

In some embodiments of the invention, glycine is present at a concentration of 6.0-9.0 mg/ml.

In some embodiments of the invention, glycine is present at a concentration of 6.5-8.5 mg/ml.

In some embodiments of the invention, glycine is present at a concentration of 7.0-8.0 mg/ml.

In some embodiments of the invention, glycine is present at a concentration of 7.51 mg/ml.

In some embodiments of the invention, methionine is present at a concentration of 0.001-5.8 mg/ml.

In some embodiments of the invention, methionine is present at a concentration of 1.1-5.8 mg/ml.

In some embodiments of the invention, methionine is present at a concentration of 1.1-1.9 mg/ml or 3.2-5.8 mg/ml.

In some embodiments of the invention, methionine is present at a concentration of 1.49 mg/ml or 4.48 mg/ml.

In some embodiments of the invention, the surfactant is present at a concentration of 0.001-6.0 mg/ml.

In some embodiments of the invention, the surfactant is present at a concentration of 0.001-4.0 mg/ml.

In some embodiments of the invention, the surfactant is present at a concentration of 0.001-3.0 mg/ml.

In some embodiments of the invention, the surfactant is present at a concentration of 0.1-2.0 mg/ml.

In some embodiments of the invention, the surfactant is present at a concentration of 0.35-1.3 mg/ml.

In some embodiments of the invention, the surfactant is poloxamer 188 or polysorbate 80 or polypropylene glycol.

In some embodiments of the invention, poloxamer 188 is present at a concentration of 0.001-6.0 mg/ml.

In some embodiments of the invention, poloxamer 188 is present at a concentration of 0.001-4.0 mg/ml.

In some embodiments of the invention, poloxamer 188 is present at a concentration of 0.001-2.5 mg/ml.

In some embodiments of the invention, poloxamer 188 is present at a concentration of 0.35-1.3 mg/ml.

In some embodiments of the invention, poloxamer 188 is present at a concentration of 0.35-0.65 mg/ml or 0.7-1.3 mg/ml.

In some embodiments of the invention, poloxamer 188 is present at a concentration of 0.35-0.65 mg/ml or 0.8-1.2 mg/ml.

In some embodiments of the invention, poloxamer 188 is present at a concentration of 0.5 mg/ml or 1.0 mg/ml.

In some embodiments of the invention, polysorbate 80 is present at a concentration of 0.001-5.0 mg/ml.

In some embodiments of the invention, polysorbate 80 is present at a concentration of 0.001-3.5 mg/ml.

In some embodiments of the invention, polysorbate 80 is present at a concentration of 0.001-2.5 mg/ml.

In some embodiments of the invention, polysorbate 80 is present at a concentration of 0.7-1.3 mg/ml.

In some embodiments of the invention, polysorbate 80 is present at a concentration of 0.8-1.2 mg/ml.

In some embodiments of the invention, polysorbate 80 is present at a concentration of 1.0 mg/ml.

In some embodiments of the invention, the pharmaceutical
composition comprises:

| | |
|---|---|
| (i) anti-TRBV9 antibody; | |
| (ii) histidine buffer or acetate buffer; | |
| (iii) osmotic agent | 0.001-200.0 mg/ml; |
| (iv) water for injection | to 1 ml. |

In some embodiments of the invention, the pharmaceutical
composition comprises:

| | |
|---|---|
| (i) anti-TRBV9 antibody; | |
| (ii) histidine buffer or acetate buffer; | |
| (iii) osmotic agent | 0.001-130.0 mg/ml; |
| (iv) water for injection | to 1 ml. |

In some embodiments of the invention, the pharmaceutical
composition comprises:

| | |
|---|---|
| (i) anti-TRBV9 antibody | 0.5-300.0 mg/ml; |
| (ii) histidine buffer or acetate buffer; | |
| (iii) osmotic agent | 0.001-200.0 mg/ml; |
| (iv) water for injection | to 1 ml. |

In some embodiments of the invention, the pharmaceutical
composition comprises:

| | |
|---|---|
| (i) anti-TRBV9 antibody | 0.5-300.0 mg/ml; |
| (ii) histidine buffer or acetate buffer; | |
| (iii) osmotic agent | 0.001-130.0 mg/ml; |
| (iv) water for injection | to 1 ml. |

In some embodiments of the invention, the pharmaceutical
composition comprises:

(i) anti-TRBV9 antibody;
(ii) histidine buffer or acetate buffer,
(iii) osmotic agent;
(iv) stabilizer;
(v) water for injection.

In some embodiments of the invention, the pharmaceutical
composition comprises:

| | |
|---|---|
| (i) anti-TRBV9 antibody; | 0.5-300.0 mg/ml; |
| (ii) histidine buffer or acetate buffer, | |
| (iii) osmotic agent; | |
| (iv) stabilizer; | |
| (v) water for injection | to 1 ml. |

In some embodiments of the invention, the pharmaceutical
composition comprises:

| | |
|---|---|
| (i) anti-TRBV9 antibody; | 0.5-300.0 mg/ml; |
| (ii) histidine buffer or acetate buffer, | |
| (iii) osmotic agent; | |
| (iv) stabilizer; | 0.001-100.0 mg/ml; |
| (v)water for injection | to 1 ml. |

In some embodiments of the invention, the pharmaceutical
composition comprises:

| | |
|---|---|
| (i) anti-TRBV9 antibody; | 0.5-300.0 mg/ml; |
| (ii) histidine buffer or acetate buffer, | |
| (iii) osmotic agent; | |
| (iv) stabilizer; | 0.35-9.5 mg/ml; |
| (v) water for injection | to 1 ml. |

In some embodiments of the invention, the pharmaceutical
composition comprises:

| | |
|---|---|
| (i) anti-TRBV9 antibody; | |
| (ii) histidine buffer or acetate buffer, | |
| (iii) osmotic agent; | 0.001-130.0 mg/ml; |
| (iv) stabilizer | 0.35-9.5 mg/ml; |
| (v) water for injection | to 1 ml. |

In some embodiments of the invention, the pharmaceutical
composition comprises:

| | |
|---|---|
| (i) anti-TRBV9 antibody | 0.5-300.0 mg/ml; |
| (ii) histidine buffer or acetate buffer, | |
| (iii) osmotic agent | 0.001-200.0 mg/ml; |
| (iv) stabilizer | 0.35-9.5 mg/ml; |
| (v) water for injection | to 1 ml. |

In some embodiments of the invention, the pharmaceutical
composition comprises:

| | |
|---|---|
| (i) anti-TRBV9 antibody | 0.5-300.0 mg/ml; |
| (ii) histidine buffer or acetate buffer, | |
| (iii) osmotic agent | 0.001-130.0 mg/ml; |
| (iv) stabilizer | 0.35-9.5 mg/ml; |
| (v) water for injection | to 1 ml. |

In some embodiments of the invention, the pharmaceutical
composition comprises:

| | |
|---|---|
| (i) anti-TRBV9 antibody; | |
| (ii) histidine buffer or acetate buffer | |
| (iii) osmotic agent being proline; | |
| (iv) water for injection | to 1 ml. |

In some embodiments of the invention, the pharmaceutical
composition comprises:

| | |
|---|---|
| (i) anti-TRBV9 antibody; | |
| (ii) histidine buffer or acetate buffer | |
| (iii) osmotic agent being proline | 14.0-32.0 mg/ml; |
| (iv) water for injection | to 1 ml. |

In some embodiments of the invention, the pharmaceutical
composition comprises:

| | |
|---|---|
| (i) anti-TRBV9 antibody; | 0.5-300.0 mg/ml; |
| (ii) histidine buffer or acetate buffer | |
| (iii) osmotic agent being proline | 14.0-32.0 mg/ml; |
| (iv) water for injection | to 1 ml. |

In some embodiments of the invention, the pharmaceutical
composition comprises:

| | |
|---|---|
| (i) anti-TRBV9 antibody; | |
| (ii) histidine buffer being a mixture of | |
| histidine | 0.4-1.0 mg/ml and |
| histidine hydrochloride monohydrate | 0.08-4.2 mg/ml; |
| (iii) osmotic agent being proline | 14.0-32.0 mg/ml; |
| (iv) water for injection | to 1 ml. |

In some embodiments of the invention, the pharmaceutical
composition comprises:

| | |
|---|---|
| (i) anti-TRBV9 antibody | 0.5-300.0 mg/ml; |
| (ii) histidine buffer being a mixture of | |
| histidine | 0.4-1.0 mg/ml and |
| histidine hydrochloride monohydrate | 0.08-4.2 mg/ml; |
| (iii) osmotic agent being proline | 14.0-32.0 mg/ml; |
| (iv) water for injection | to 1 ml. |

In some embodiments of the invention, the pharmaceutical
composition comprises:

| | |
|---|---|
| (i) anti-TRBV9 antibody | 5-125.0 mg/ml; |
| (ii) histidine buffer being a mixture of | |
| histidine | 0.4-1.0 mg/ml and |
| histidine hydrochloride monohydrate | 0.08-4.2 mg/ml; |
| (iii) osmotic agent being proline | 14.0-32.0 mg/ml; |
| (iv) water for injection | to 1 ml. |

In some embodiments of the invention, the pharmaceutical
composition comprises:

| | |
|---|---|
| (i) anti-TRBV9 antibody | 5-100.0 mg/ml; |
| (ii) histidine buffer being a mixture of | |
| histidine | 0.4-1.0 mg/ml and |
| histidine hydrochloride monohydrate | 0.08-4.2 mg/ml; |
| (iii) osmotic agent being proline | 14.0-32.0 mg/ml; |
| (iv) water for injection | to 1 ml. |

In some embodiments of the invention, the pharmaceutical
composition comprises:

| | |
|---|---|
| (i) anti-TRBV9 antibody; | |
| (ii) histidine buffer or acetate buffer | |
| (iii) osmotic agent being proline | 27 mg/ml; |
| (iv) water for injection | to 1 ml. |
| In some embodiments of the invention, the pharmaceutical | |
| (i) anti-TRBV9 antibody; | 0.5-300.0 mg/ml; |
| (ii) histidine buffer or acetate buffer | |
| (iii) osmotic agent being proline | 27 mg/ml; |
| (iv) water for injection to 1 ml. | |

In some embodiments of the invention, the pharmaceutical
composition comprises:

| | |
|---|---|
| (i) anti-TRBV9 antibody; | |
| (ii) histidine buffer being a mixture of | 0.517 mg/ml and |
| histidine | 0.350 mg/ml; |
| histidine hydrochloride monohydrate | |
| (iii) osmotic agent being proline | 27 mg/ml; |
| (iv) water for injection | to 1 ml. |

In some embodiments of the invention, the pharmaceutical
composition comprises:

| | |
|---|---|
| (i) anti-TRBV9 antibody | 0.5-300.0 mg/ml; |
| (ii) histidine buffer being a mixture of | 0.517 mg/ml and |
| histidine | 0.350 mg/ml; |
| histidine hydrochloride monohydrate | |
| (iii) osmotic agent being proline | 27 mg/ml; |
| (iv) water for injection | to 1 ml. |

In some embodiments of the invention, the pharmaceutical
composition comprises:

| | |
|---|---|
| (i) anti-TRBV9 antibody | 25 mg/ml; |
| (ii) histidine buffer or acetate buffer | |
| (iii) osmotic agent being proline | 27 mg/ml; |
| (iv) water for injection | to 1 ml. |

In some embodiments of the invention, the pharmaceutical
composition comprises:

| | |
|---|---|
| (i) anti-TRBV9 antibody | 25 mg/ml; |
| (ii) histidine buffer being a mixture of | 0.517 mg/ml and |
| histidine | 0.350 mg/ml; |
| histidine hydrochloride monohydrate | |
| (iii) osmotic agent being proline | 27 mg/ml; |
| (iv) water for injection | to 1 ml. |

In some embodiments of the invention, the pharmaceutical
composition comprises:

| | |
|---|---|
| (i) anti-TRBV9 antibody; | |
| (ii) histidine buffer or acetate buffer | |
| (iii) osmotic agent being proline | 19 mg/ml; |
| (iv) water for injection | to 1 ml. |

In some embodiments of the invention, the pharmaceutical
composition comprises:

| | |
|---|---|
| (i) anti-TRBV9 antibody; | 0.5-300.0 mg/ml; |
| (ii) histidine buffer or acetate buffer | |
| (iii) osmotic agent being proline | 19 mg/ml; |
| (iv) water for injection | to 1 ml. |

In some embodiments of the invention, the pharmaceutical
composition comprises:

| | |
|---|---|
| (i) anti-TRBV9 antibody; | 25.0 mg/ml; |
| (ii) histidine buffer or acetate buffer | |
| (iii) osmotic agent being proline | 19 mg/ml; |
| (iv) water for injection | to 1 ml. |

In some embodiments of the invention, the pharmaceutical
composition comprises:

| | |
|---|---|
| (i) anti-TRBV9 antibody; | |
| (ii) histidine buffer being a mixture of | 0.689 mg/ml and |
| histidine | 0.117 mg/ml; |
| histidine hydrochloride monohydrate | |
| (iii) osmotic agent being proline | 19 mg/ml; |
| (iv) water for injection | to 1 ml. |

In some embodiments of the invention, the pharmaceutical
composition comprises:

| | |
|---|---|
| (i) anti-TRBV9 antibody | 0.5-300.0 mg/ml; |
| (ii) histidine buffer being a mixture of | 0.689 mg/ml and |
| histidine | 0.117 mg/ml; |
| histidine hydrochloride monohydrate | |
| (iii)osmotic agent being proline | 19 mg/ml; |
| (iv) water for injection | |
| to 1 ml. | |

In some embodiments of the invention, the pharmaceutical
composition comprises:

| | |
|---|---|
| (i) anti-TRBV9 antibody | 25.0 mg/ml; |
| (ii) histidine buffer being a mixture of | 0.689 mg/ml and |
| histidine | 0.117 mg/ml; |
| histidine hydrochloride monohydrate | |
| (iii) osmotic agent being proline | 19 mg/ml; |
| (iv) water for injection | to 1 ml. |

In some embodiments of the invention, the pharmaceutical
composition comprises:

(i) anti-TRBV9 antibody;
(ii) histidine buffer or acetate buffer,
(iii) osmotic agent being proline;
(iv) stabilizer being glycine;
(v) water for injection.

In some embodiments of the invention, the pharmaceutical
composition comprises:

| | |
|---|---|
| (i) anti-TRBV9 antibody; | |
| (ii) histidine buffer or acetate buffer, | |
| (iii) osmotic agent being proline | |
| (iv) stabilizer being glycine | 0.001-100.0 mg/ml; |
| (v) water for injection | to 1 ml. |

In some embodiments of the invention, the pharmaceutical
composition comprises:

| | |
|---|---|
| (i) anti-TRBV9 antibody; | |
| (ii) histidine buffer or acetate buffer, | |
| (iii) osmotic agent being proline | |
| (iv) stabilizer being glycine | 5.5-9.5 mg/ml; |
| (v) water for injection | to 1 ml. |

In some embodiments of the invention, the pharmaceutical
composition comprises:

| | |
|---|---|
| (i) anti-TRBV9 antibody; | |
| (ii) histidine buffer or acetate buffer, | |
| (iii) osmotic agent being proline | 14.0-32.0 mg/ml; |
| (iv) stabilizer being glycine | 5.5-9.5 mg/ml; |
| (v) water for injection | to 1 ml. |

In some embodiments of the invention, the pharmaceutical
composition comprises:

| | |
|---|---|
| (i) anti-TRBV9 antibody; | 0.5-300.0 mg/ml; |
| (ii) histidine buffer or acetate buffer, | |
| (iii) osmotic agent being proline | 14.0-32.0 mg/ml; |
| (iv) stabilizer being glycine | 5.5-9.5 mg/ml; |
| (v) water for injection | to 1 ml. |

In some embodiments of the invention, the pharmaceutical
composition comprises:

| | |
|---|---|
| (i) anti-TRBV9 antibody; | 0.5-125.0 mg/ml; |
| (ii) histidine buffer or acetate buffer, | |
| (iii) osmotic agent being proline | 14.0-32.0 mg/ml; |
| (iv) stabilizer being glycine | 5.5-9.5 mg/ml; |
| (v) water for injection | to 1 ml. |

In some embodiments of the invention, the pharmaceutical
composition comprises:

| | |
|---|---|
| (i) anti-TRBV9 antibody; | 0.5-100.0 mg/ml; |
| (ii) histidine buffer or acetate buffer, | |
| (iii) osmotic agent being proline | 14.0-32.0 mg/ml; |
| (iv) stabilizer being glycine | 5.5-9.5 mg/ml; |
| (v) water for injection | to 1 ml. |

In some embodiments of the invention, the pharmaceutical
composition comprises:

| | |
|---|---|
| (i) anti-TRBV9 antibody; | |
| (ii) histidine buffer being a mixture of | 0.4-1.0 mg/ml and |
| histidine | 0.08-4.2 mg/ml; |
| histidine hydrochloride monohydrate | |
| (iii) osmotic agent being proline | 14.0-32.0 mg/ml; |
| (iv) stabilizer being glycine | 5.5-9.5 mg/ml; |
| (v) water for injection | to 1 ml. |

In some embodiments of the invention, the pharmaceutical
composition comprises:

| | |
|---|---|
| (i) anti-TRBV9 antibody | 0.5-300.0 mg/ml; |
| (ii) histidine buffer being a mixture of | 0.4-1.0 mg/ml and |
| histidine | 0.08-4.2 mg/ml; |
| histidine hydrochloride monohydrate | |
| (iii) osmotic agent being proline | 14.0-32.0 mg/ml; |
| (iv) stabilizer being glycine | 5.5-9.5 mg/ml; |
| (v) water for injection | to 1 ml. |

In some embodiments of the invention, the pharmaceutical
composition comprises:

| | |
|---|---|
| (i) anti-TRBV9 antibody | 5-125.0 mg/ml; |
| (ii) histidine buffer being a mixture of | 0.4-1.0 mg/ml and |
| histidine | 0.08-4.2 mg/ml; |
| histidine hydrochloride monohydrate | |
| (iii) osmotic agent being proline | 14.0-32.0 mg/ml; |
| (iv) stabilizer being glycine | 5.5-9.5 mg/ml; |
| (v) water for injection | to 1 ml. |

In some embodiments of the invention, the pharmaceutical
composition comprises:

| | |
|---|---|
| (i) anti-TRBV9 antibody | 5-100.0 mg/ml; |
| (ii) histidine buffer being a mixture of | 0.4-1.0 mg/ml and |
| histidine | 0.08-4.2 mg/ml; |
| histidine hydrochloride monohydrate | |
| (iii) osmotic agent being proline | 14.0-32.0 mg/ml; |
| (iv) stabilizer being glycine | 5.5-9.5 mg/ml; |
| (v) water for injection | to 1 ml. |

In some embodiments of the invention, the pharmaceutical

composition comprises:

| | |
|---|---|
| (i) anti-TRBV9 antibody | 25.0 mg/ml; |
| (ii) histidine buffer being a mixture of | 0.4-1.0 mg/ml and |
| histidine | 0.08-4.2 mg/ml; |
| histidine hydrochloride monohydrate | |
| (iii) osmotic agent being proline | 14.0-32.0 mg/ml; |
| (iv) stabilizer being glycine | 5.5-9.5 mg/ml; |
| (v) water for injection | to 1 ml. |

In some embodiments of the invention, the pharmaceutical
composition comprises:

| | |
|---|---|
| (i) anti-TRBV9 antibody; | |
| (ii) histidine buffer or acetate buffer, | |
| (iii) osmotic agent being proline | 21 mg/ml; |
| (iv) stabilizer being glycine | 7.51 mg/ml; |
| (v) water for injection | to 1 ml. |

In some embodiments of the invention, the pharmaceutical
composition comprises:

| | |
|---|---|
| (i) anti-TRBV9 antibody; | 0.5-300.0 mg/ml; |
| (ii) histidine buffer or acetate buffer, | |
| (iii) osmotic agent being proline | 21 mg/ml; |
| (iv) stabilizer being glycine | 7.51 mg/ml; |
| (v) water for injection | to 1 ml. |

In some embodiments of the invention, the pharmaceutical
composition comprises:

| | |
|---|---|
| (i) anti-TRBV9 antibody; | |
| (ii) histidine buffer being a mixture of | 0.517 mg/ml and |
| histidine | 0.350 mg/ml; |
| histidine hydrochloride monohydrate | |
| (iii) osmotic agent being proline | 21 mg/ml; |
| (iv) stabilizer being glycine | 7.51 mg/ml; |
| (v) water for injection | to 1 ml. |

In some embodiments of the invention, the pharmaceutical
composition comprises

| | |
|---|---|
| (i) anti-TRBV9 antibody | 0.5-300.0 mg/ml; |
| (ii) histidine buffer being a mixture of | 0.517 mg/ml and |
| histidine | 0.350 mg/ml; |
| histidine hydrochloride monohydrate | |
| (iii) osmotic agent being proline | 21 mg/ml; |
| (iv) stabilizer being glycine | 7.51 mg/ml; |
| (v) water for injection | to 1 ml. |

In some embodiments of the invention, the pharmaceutical
composition comprises

| | |
|---|---|
| (i) anti-TRBV9 antibody | 25 mg/ml; |
| (ii) histidine buffer being a mixture of | 0.517 mg/ml and |
| histidine | 0.350 mg/ml; |
| histidine hydrochloride monohydrate | |
| (iii) osmotic agent being proline | 21 mg/ml; |
| (iv) stabilizer being glycine | 7.51 mg/ml; |
| (v) water for injection | to 1 ml. |

In some embodiments of the invention, the pharmaceutical
composition comprises:

(i) anti-TRBV9 antibody;
(ii) histidine buffer or acetate buffer;
(iii) osmotic agent being proline;
(iv) stabilizer being poloxamer 188;
(v) water for injection.

In some embodiments of the invention, the pharmaceutical
composition comprises:

| | |
|---|---|
| (i) anti-TRBV9 antibody; | |
| (ii) histidine buffer or acetate buffer; | |
| (iii) osmotic agent being proline | |
| (iv) stabilizer being poloxamer 188 | 0.35-1.3 mg/ml; |
| (v) water for injection | to 1 ml. |

In some embodiments of the invention, the pharmaceutical
composition comprises:

| | |
|---|---|
| (i) anti-TRBV9 antibody; | |
| (ii) histidine buffer or acetate buffer; | |
| (iii) osmotic agent being proline | 14.0-32.0 mg/ml; |
| (iv) stabilizer being poloxamer 188 | 0.35-1.3 mg/ml; |
| (v) water for injection | to 1 ml. |

In some embodiments of the invention, the pharmaceutical
composition comprises:

| | |
|---|---|
| (i) anti-TRBV9 antibody; | 0.5-300.0 mg/ml; |
| (ii) histidine buffer or acetate buffer; | |
| (iii) osmotic agent being proline | 14.0-32.0 mg/ml; |
| (iv) stabilizer being poloxamer 188 | 0.35-1.3 mg/ml; |
| (v) water for injection | to 1 ml. |

In some embodiments of the invention, the pharmaceutical
composition comprises:

| | |
|---|---|
| (i) anti-TRBV9 antibody; | |
| (ii) histidine buffer being a mixture of | 0.4-1.0 mg/ml and |
| histidine | 0.08-4.2 mg/ml; |
| histidine hydrochloride monohydrate | |
| (iii) osmotic agent being proline | 14.0-32.0 mg/ml; |
| (iv) stabilizer being poloxamer 188 | 0.35-1.3 mg/ml; |
| (v) water for injection | to 1 ml. |

In some embodiments of the invention, the pharmaceutical
composition comprises:

| | |
|---|---|
| (i) anti-TRBV9 antibody | 0.5-300.0 mg/ml; |
| (ii) histidine buffer being a mixture of | 0.4-1.0 mg/ml and |
| histidine | 0.08-4.2 mg/ml; |
| histidine hydrochloride monohydrate | |
| (iii) smotic agent being proline | 14.0-32.0 mg/ml; |
| (iv) stabilizer being poloxamer 188 | 0.35-1.3 mg/ml; |
| (v) water for injection | to 1 ml. |

In some embodiments of the invention, the pharmaceutical
composition comprises:

| | |
|---|---|
| (i) anti-TRBV9 antibody | 5.0-125.0 mg/ml; |
| (ii) histidine buffer being a mixture of | 0.4-1.0 mg/ml and |
| histidine | 0.08-4.2 mg/ml; |
| histidine hydrochloride monohydrate | |
| (iii) osmotic agent being proline | 14.0-32.0 mg/ml; |
| (iv) stabilizer being poloxamer 188 | 0.35-1.3 mg/ml; |
| (v) water for injection | to 1 ml. |

In some embodiments of the invention, the pharmaceutical
composition comprises:

| | |
|---|---|
| (i) anti-TRBV9 antibody | 5.0-100.0 mg/ml; |
| (ii) histidine buffer being a mixture of | 0.4-1.0 mg/ml and |
| histidine | 0.08-4.2 mg/ml; |
| histidine hydrochloride monohydrate | |
| (iii) osmotic agent being proline | 14.0-32.0 mg/ml; |
| (iv) stabilizer being poloxamer 188 | 0.35-1.3 mg/ml; |
| (v) water for injection | to 1 ml. |

In some embodiments of the invention, the pharmaceutical
composition comprises:

| | |
|---|---|
| (i) anti-TRBV9 antibody | 0.5-300.0 mg/ml; |
| (ii) histidine buffer or acetate buffer; | |
| (iii) osmotic agent being proline | 9.0 mg/ml; |
| (iv) stabilizer being poloxamer 188 | 0.5 mg/ml; |
| (v) water for injection | to 1 ml. |

In some embodiments of the invention, the pharmaceutical
composition comprises:

| | |
|---|---|
| (i) anti-TRBV9 antibody; | |
| (ii) histidine buffer being a mixture of | 0.517 mg/ml and |
| histidine | 0.350 mg/ml; |
| histidine hydrochloride monohydrate | |
| (iii) osmotic agent being proline | 9.0 mg/ml; |
| (iv) stabilizer being poloxamer 188 | 0.5 mg/ml; |
| (v) water for injection | to 1 ml. |

In some embodiments of the invention, the pharmaceutical
composition comprises:

| | |
|---|---|
| (i) anti-TRBV9 antibody | 0.5-300.0 mg/ml; |
| (ii) histidine buffer being a mixture of | 0.517 mg/ml and |
| histidine | 0.350 mg/ml; |
| histidine hydrochloride monohydrate | |
| (iii) osmotic agent being proline | 9.0 mg/ml; |
| (iv) stabilizer being poloxamer 188 | 0.5 mg/ml; |
| (v) water for injection | to 1 ml. |

In some embodiments of the invention, the pharmaceutical

composition comprises:

| | |
|---|---|
| (i) anti-TRBV9 antibody | 25.0 mg/ml; |
| (ii) histidine buffer being a mixture of | 0.517 mg/ml and |
| histidine | 0.350 mg/ml; |
| histidine hydrochloride monohydrate | |
| (iii) osmotic agent being proline | 9.0 mg/ml; |
| (iv) stabilizer being poloxamer 188 | 0.5 mg/ml; |
| (v) water for injection | to 1 ml. |

In some embodiments of the invention, the pharmaceutical

composition comprises:

| | |
|---|---|
| (i) anti-TRBV9 antibody; | |
| (ii) histidine buffer being a mixture of | 0.517 mg/ml and |
| histidine | 0.350 mg/ml; |
| histidine hydrochloride monohydrate | |
| (iii) osmotic agent being proline | 9.0 mg/ml; |
| (iv) stabilizer being poloxamer 188 | 1.0 mg/ml; |
| (v) water for injection | to 1 ml. |

In some embodiments of the invention, the pharmaceutical
composition comprises:

| | |
|---|---|
| (i) anti-TRBV9 antibody | 25.0 mg/ml; |
| (ii) histidine buffer being a mixture of | 0.517 mg/ml and |
| histidine | 0.350 mg/ml; |
| histidine hydrochloride monohydrate | |
| (iii) osmotic agent being proline | 9.0 mg/ml; |
| (iv) stabilizer being poloxamer 188 | 1.0 mg/ml; |
| (v) water for injection | to 1 ml. |

In some embodiments of the invention, the pharmaceutical
composition comprises:

(1) anti-TRBV9 antibody;
(ii) histidine buffer or acetate buffer;
(iii) osmotic agent being sorbitol;
(iv) water for injection.

In some embodiments of the invention, the pharmaceutical
composition comprises:

| | |
|---|---|
| (i) anti-TRBV9 antibody; | |
| (ii) histidine buffer or acetate buffer; | |
| (iii) osmotic agent being sorbitol | |
| | 35.0-65.0 mg/ml; |
| (iv) water for injection | to 1 ml. |

In some embodiments of the invention, the pharmaceutical
composition comprises:

| | |
|---|---|
| (i) anti-TRBV9 antibody; | 0.5-300.0 mg/ml; |
| (ii) histidine buffer or acetate buffer; | |
| (iii) osmotic agent being sorbitol | 35.0-65.0 mg/ml; |
| (iv) water for injection | to 1 ml. |

In some embodiments of the invention, the pharmaceutical
composition comprises:

| | |
|---|---|
| (i) anti-TRBV9 antibody; | |
| (ii) histidine buffer being a mixture of | 0.4-1.0 mg/ml and |
| histidine | 0.08-4.2 mg/ml; |
| histidine hydrochloride monohydrate | |
| (iii) osmotic agent being sorbitol | 35.0-65.0 mg/ml; |
| (iv) water for injection | to 1 ml. |

In some embodiments of the invention, the pharmaceutical
composition comprises:

| | |
|---|---|
| (i) anti-TRBV9 antibody; | 0.5-300.0 mg/ml; |
| (ii) histidine buffer or acetate buffer; | |
| (iii) osmotic agent being sorbitol | 50.0 mg/ml; |
| (iv) water for injection | to 1 ml. |

In some embodiments of the invention, the pharmaceutical

composition comprises:

| | |
|---|---|
| (i) anti-TRBV9 antibody; | |
| (ii) histidine buffer being a mixture of | 0.517 mg/ml and |
| histidine | 0.350 mg/ml; |
| histidine hydrochloride monohydrate | |
| (iii) osmotic agent being sorbitol | 50.0 mg/ml; |
| (iv) water for injection | to 1 ml. |

In some embodiments of the invention, the pharmaceutical
composition comprises:

| | |
|---|---|
| (i) anti-TRBV9 antibody | 0.5-300.0 mg/ml; |
| (ii) histidine buffer being a mixture of | 0.517 mg/ml and |
| histidine | 0.350 mg/ml; |
| histidine hydrochloride monohydrate | |
| (iii) osmotic agent being sorbitol | 50.0 mg/ml; |
| (iv) water for injection | to 1 ml. |

n some embodiments of the invention, the pharmaceutical
composition comprises:

| | |
|---|---|
| (i) anti-TRBV9 antibody | 25.0 mg/ml; |
| (ii) histidine buffer being a mixture of | 0.517 mg/ml and |
| histidine | 0.350 mg/ml; |
| histidine hydrochloride monohydrate | |
| (iii) osmotic agent being sorbitol | 50.0 mg/ml; |
| (iv) water for injection | to 1 ml. |

In some embodiments of the invention, the pharmaceutical
composition comprises:

| | |
|---|---|
| (i) anti-TRBV9 antibody; | |
| (ii) histidine buffer or acetate buffer; | |
| (iii) osmotic agent being sorbitol | |
| (iv) stabilizer being methionine | 1.1-5.8 mg/ml; |
| (v) water for injection | to 1 ml. |

In some embodiments of the invention, the pharmaceutical
composition comprises:

| | |
|---|---|
| (i) anti-TRBV9 antibody; | |
| (ii) histidine buffer or acetate buffer; | |
| (iii) osmotic agent being sorbitol | 35.0-65.0 mg/ml; |
| (iv) stabilizer being methionine | 1.1-5.8 mg/ml; |
| (v) water for injection | to 1 ml. |

In some embodiments of the invention, the pharmaceutical
composition comprises:

| | |
|---|---|
| (i) anti-TRBV9 antibody | 0.5-300.0 mg/ml; |
| (ii) histidine buffer or acetate buffer; | |
| (iii) osmotic agent being sorbitol | 35.0-65.0 mg/ml; |
| (iv) stabilizer being methionine | 1.1-5.8 mg/ml; |
| (v) water for injection | to 1 ml. |

In some embodiments of the invention, the pharmaceutical
composition comprises:

| | |
|---|---|
| (i) anti-TRBV9 antibody; | |
| (ii) histidine buffer being a mixture of | 0.4-1.0 mg/ml and |
| histidine | 0.08-4.2 mg/ml; |
| histidine hydrochloride monohydrate | |
| (iii) osmotic agent being sorbitol | 35.0-65.0 mg/ml; |
| (iv) stabilizer being methionine | 1.1-5.8 mg/ml; |
| (v) water for injection | to 1 ml. |

In some embodiments of the invention, the pharmaceutical

composition comprises:

| | |
|---|---|
| (i) anti-TRBV9 antibody; | |
| (ii) histidine buffer being a mixture of | 0.517 mg/ml and |
| histidine | 0.350 mg/ml; |
| histidine hydrochloride monohydrate | |
| (iii) osmotic agent being sorbitol | 50.0 mg/ml; |
| (iv) stabilizer being methionine | 1.49 mg/ml; |
| (v) water for injection | to 1 ml. |

In some embodiments of the invention, the pharmaceutical
composition comprises:

| | |
|---|---|
| (i) anti-TRBV9 antibody | 0.05-300.0 mg/ml; |
| (ii) histidine buffer being a mixture of | 0.517 mg/ml and |
| histidine | 0.350 mg/ml; |
| histidine hydrochloride monohydrate | |
| (iii) osmotic agent being sorbitol | 50.0 mg/ml; |
| (iv) stabilizer being methionine | 1.49 mg/ml; |
| (v) water for injection | to 1 ml. |

In some embodiments of the invention, the pharmaceutical
composition comprises:

| | |
|---|---|
| (i) anti-TRBV9 antibody | 25.0 mg/ml; |
| (ii) histidine buffer being a mixture of | 0.517 mg/ml and |
| histidine | 0.350 mg/ml; |
| histidine hydrochloride monohydrate | |
| (iii) osmotic agent being sorbitol | 50.0 mg/ml; |
| (iv) stabilizer being methionine | 1.49 mg/ml; |
| (v) water for injection | to 1 ml. |

In some embodiments of the invention, the pharmaceutical

composition comprises:

| | |
|---|---|
| (i) anti-TRBV9 antibody; | |
| (ii) histidine buffer being a mixture of | 0.517 mg/ml and |
| histidine | 0.350 mg/ml; |
| histidine hydrochloride monohydrate | |
| (iii) osmotic agent being sorbitol | 50.0 mg/ml; |
| (iv) stabilizer being methionine | 4.48 mg/ml; |
| (v) water for injection | to 1 ml. |

In some embodiments of the invention, the pharmaceutical

composition comprises:

| | |
|---|---|
| (i) anti-TRBV9 antibody | 0.5-300.0 mg/ml; |
| (ii) histidine buffer being a mixture of | 0.517 mg/ml and |
| histidine | 0.350 mg/ml; |
| histidine hydrochloride monohydrate | |
| (iii) osmotic agent being sorbitol | 50.0 mg/ml; |
| (iv) stabilizer being methionine | 4.48 mg/ml; |
| (v) water for injection | to 1 ml. |

In some embodiments of the invention, the pharmaceutical

composition comprises:

| | |
|---|---|
| (i) anti-TRBV9 antibody | 25.0 mg/ml; |
| (ii) histidine buffer being a mixture of | 0.517 mg/ml and |
| histidine | 0.350 mg/ml; |
| histidine hydrochloride monohydrate | |
| (iii) osmotic agent being sorbitol | 50.0 mg/ml; |
| (iv) stabilizer being methionine | 4.48 mg/ml; |
| (v) water for injection | to 1 ml. |

In some embodiments of the invention, the pharmaceutical

composition comprises:

(i) anti-TRBV9 antibody;
(ii) histidine buffer or acetate buffer;
(iii) osmotic agent being trehalose;
(iv) water for injection.

In some embodiments of the invention, the pharmaceutical

composition comprises:

| | |
|---|---|
| (i) anti-TRBV9 antibody; | |
| (ii) histidine buffer or acetate buffer; | |
| (iii) osmotic agent being trehalose | 0.001-200.0 mg/ml; |
| (iv) water for injection | to 1 ml. |

In some embodiments of the invention, the pharmaceutical

composition comprises:

| | |
|---|---|
| (i) anti-TRBV9 antibody; | |
| (ii) histidine buffer or acetate buffer; | |
| (iii) osmotic agent being trehalose | 70-130.0 mg/ml; |
| (iv) water for injection | to 1 ml. |
| In some embodiments of the invention, the pharmaceutical | |

composition comprises:
(i) anti-TRBV9 antibody; 0.5-300.0 mg/ml;

| | |
|---|---|
| (ii) histidine buffer or acetate buffer; | |
| (iii) osmotic agent being trehalose | 70-130.0 mg/ml; |
| (iv) water for injection | to 1 ml. |

In some embodiments of the invention, the pharmaceutical
composition comprises:

| | |
|---|---|
| (i) anti-TRBV9 antibody; | |
| (ii) histidine buffer being a mixture of | 0.4-1.0 mg/ml and |
| histidine | |
| histidine hydrochloride monohydrate | 0.08-4.2 mg/ml; |
| (iii) osmotic agent being trehalose | 70-130.0 mg/ml; |
| (iv) water for injection | to 1 ml. |

In some embodiments of the invention, the pharmaceutical
composition comprises:

| | |
|---|---|
| (i) anti-TRBV9 antibody | 0.5-300.0 mg/ml; |
| (ii) histidine buffer being a mixture of | |
| histidine | 0.4-1.0 mg/ml and |
| histidine hydrochloride monohydrate | 0.08-4.2 mg/ml; |
| (iii) osmotic agent being trehalose | 70-130.0 mg/ml; |
| (iv) water for injection | to 1 ml. |

In some embodiments of the invention, the pharmaceutical

composition comprises:

| | |
|---|---|
| (i) anti-TRBV9 antibody | 5.0-125.0 mg/ml; |
| (ii) histidine buffer being a mixture of | |
| histidine | 0.4-1.0 mg/ml and |
| histidine hydrochloride monohydrate | 0.08-4.2 mg/ml; |
| (iii) osmotic agent being trehalose | 70-130.0 mg/ml; |
| (iv) water for injection | to 1 ml. |

In some embodiments of the invention, the pharmaceutical

composition comprises:

| | |
|---|---|
| (i) anti-TRBV9 antibody | 5.0-100.0 mg/ml; |
| (ii) histidine buffer being a mixture of | |
| histidine | 0.4-1.0 mg/ml and |
| histidine hydrochloride monohydrate | 0.08-4.2 mg/ml; |
| (iii) osmotic agent being trehalose | 70-130.0 mg/ml; |
| (iv) water for injection | to 1 ml. |

In some embodiments of the invention, the pharmaceutical

composition comprises:

| | |
|---|---|
| (i) anti-TRBV9 antibody; | |
| (ii) histidine buffer being a mixture of | |
| histidine | 0.517 mg/ml |
| | and |
| histidine hydrochloride monohydrate | 0.350 mg/ml; |
| (iii) osmotic agent being trehalose | 100.0 mg/ml; |
| (iv) water for injection | to 1 ml. |

In some embodiments of the invention, the pharmaceutical

composition comprises:

| | |
|---|---|
| (i) anti-TRBV9 antibody | 0.5-300.0 mg/ml; |
| (ii) histidine buffer being a mixture of | |
| histidine | 0.517 mg/ml |
| | and |
| histidine hydrochloride monohydrate | 0.350 mg/ml; |
| (iii) osmotic agent being trehalose | 100.0 mg/ml; |
| (iv) water for injection | to 1 ml. |

In some embodiments of the invention, the pharmaceutical

composition comprises:

| | |
|---|---|
| (i) anti-TRBV9 antibody | 25.0 mg/ml; |
| (ii) histidine buffer being a mixture of | |
| histidine | 0.517 mg/ml |
| | and |
| histidine hydrochloride monohydrate | 0.350 mg/ml; |
| (iii) osmotic agent being trehalose | 100.0 mg/ml; |
| (iv) water for injection | to 1 ml. |

In some embodiments of the invention, the pharmaceutical

composition comprises:

(i) anti-TRBV9 antibody;
(ii) histidine buffer or acetate buffer;
(iii) osmotic agent being sodium chloride;
(iv) water for injection.

In some embodiments of the invention, the pharmaceutical
composition comprises:

| | |
|---|---|
| (i) anti-TRBV9 antibody; | |
| (ii) histidine buffer or acetate buffer; | |
| (iii) osmotic agent being sodium | |
| chloride | 6.0-11.5 mg/ml; |
| (iv) water for injection | to 1 ml. |

In some embodiments of the invention, the pharmaceutical
composition comprises:

| | |
|---|---|
| (i) anti-TRBV9 antibody; | 0.5-300.0 mg/ml; |
| (ii) histidine buffer or acetate | |
| buffer; | |
| (iii) osmotic agent being sodium | |
| chloride | 6.0-11.5 mg/ml; |
| (iv) water for injection | to 1 ml. |

In some embodiments of the invention, the pharmaceutical
composition comprises:

| | |
|---|---|
| (i) anti-TRBV9 antibody; | |
| (ii) histidine buffer being a mixture of | |
| histidine | 0.4-1.0 mg/ml |
| | and |
| histidine hydrochloride monohydrate | 0.08-4.2 mg/ml; |
| (iii) osmotic agent being sodium | |
| chloride | 6.0-11.5 mg/ml; |
| (iv) water for injection | to 1 ml. |

In some embodiments of the invention, the pharmaceutical
composition comprises:

| | |
|---|---|
| (i) anti-TRBV9 antibody | 0.5-300.0 mg/ml; |
| (ii) histidine buffer being a mixture of | |
| histidine | 0.4-1.0 mg/ml and |
| histidine hydrochloride monohydrate | 0.08-4.2 mg/ml; |
| (iii) osmotic agent being sodium | |
| chloride | 6.0-11.5 mg/ml; |
| (iv) water for injection | to 1 ml. |

In some embodiments of the invention, the pharmaceutical
composition comprises:

| | |
|---|---|
| (i) anti-TRBV9 antibody | 5-125.0 mg/ml; |
| (ii) histidine buffer being a mixture of | |
| histidine | 0.4-1.0 mg/ml and |
| histidine hydrochloride monohydrate | 0.08-4.2 mg/ml; |
| (iii) osmotic agent being sodium | |
| chloride | 6.0-11.5 mg/ml; |
| (iv) water for injection | to 1 ml. |

In some embodiments of the invention, the pharmaceutical
composition comprises:

| | |
|---|---|
| (i) anti-TRBV9 antibody | 5-100.0 mg/ml; |
| (ii) histidine buffer being a mixture of | |
| histidine | 0.4-1.0 mg/ml and |
| histidine hydrochloride monohydrate | 0.08-4.2 mg/ml; |
| (iii) osmotic agent being sodium | |
| chloride | 6.0-11.5 mg/ml; |
| (iv) water for injection | to 1 ml. |

In some embodiments of the invention, the pharmaceutical

composition comprises:

| | |
|---|---|
| (i) anti-TRBV9 antibody; | |
| (ii) histidine buffer being a mixture of | |
| histidine | 0.517 mg/ml |
| | and |
| histidine hydrochloride monohydrate | 0.350 mg/ml; |
| (iii) osmotic agent being sodium | |
| chloride | 9.0 mg/ml; |
| (iv) water for injection | to 1 ml. |

In some embodiments of the invention, the pharmaceutical
composition comprises:

| | |
|---|---|
| (i) anti-TRBV9 antibody | 5.0-300.0 mg/ml; |
| (ii) histidine buffer being a mixture of | |
| histidine | 0.517 mg/ml |
| | and |
| histidine hydrochloride monohydrate | 0.350 mg/ml; |
| (iii) osmotic agent being sodium | |
| chloride | 9.0 mg/ml; |
| (iv) water for injection | to 1 ml. |

In some embodiments of the invention, the pharmaceutical
composition comprises:

| | |
|---|---|
| (i) anti-TRBV9 antibody | 25.0 mg/ml; |
| (ii) histidine buffer being a mixture of | |
| histidine | 0.517 mg/ml |
| | and |
| histidine hydrochloride monohydrate | 0.350 mg/ml; |
| (iii) osmotic agent being sodium chloride | |
| | 9.0 mg/ml; |
| (iv) water for injection | to 1 ml. |

In some embodiments of the invention, the pharmaceutical
composition comprises:

(i) anti-TRBV9 antibody;
(ii) histidine buffer or acetate buffer;
(iii) osmotic agent being sodium chloride;
(iv) stabilizer being poloxamer 188 or
polysorbate 80;
(v) water for injection.

In some embodiments of the invention, the pharmaceutical
composition comprises:

| | |
|---|---|
| (i) anti-TRBV9 antibody; | |
| (ii) histidine buffer or acetate buffer; | |
| (iii) osmotic agent being sodium | |
| chloride | 6.0-11.5 mg/ml; |
| (iv) stabilizer being poloxamer 188 or | |
| polysorbate 80 | 0.35-1.3 mg/ml; |
| (v) water for injection | to 1 ml. |

In some embodiments of the invention, the pharmaceutical
composition comprises:

| | |
|---|---|
| (i) anti-TRBV9 antibody; | 0.5-300.0 mg/ml; |
| (ii) histidine buffer or acetate buffer; | |
| (iii) osmotic agent being sodium | |
| chloride | 6.0-11.5 mg/ml; |
| (iv) stabilizer being poloxamer 188 or | |
| polysorbate 80 | 0.35-1.3 mg/ml; |
| (v) water for injection | to 1 ml. |

In some embodiments of the invention, the pharmaceutical
composition comprises:

| | |
|---|---|
| (i) anti-TRBV9 antibody; | |
| (ii) histidine buffer being a mixture of | |
| histidine | 0.4-1.0 mg/ml |
| | and |
| histidine hydrochloride monohydrate | 0.08-4.2 mg/ml; |
| (iii) osmotic agent being sodium | |
| chloride | 6.0-11.5 mg/ml; |
| (iv) stabilizer being poloxamer 188 or | |
| polysorbate 80 | 0.35-1.3 mg/ml; |
| (v) water for injection | to 1 ml. |

In some embodiments of the invention, the pharmaceutical
composition comprises:

| | |
|---|---|
| (i) anti-TRBV9 antibody | 0.5-300.0 mg/ml; |
| (ii) histidine buffer being a mixture of | |
| histidine | 0.4-1.0 mg/ml and |
| histidine hydrochloride monohydrate | 0.08-4.2 mg/ml; |
| (iii) osmotic agent being sodium | |
| chloride | 6.0-11.5 mg/ml; |
| (iv) stabilizer being poloxamer 188 or | |
| polysorbate 80 | 0.35-1.3 mg/ml; |
| (v) water for injection | to 1 ml. |

In some embodiments of the invention, the pharmaceutical
composition comprises:

| | |
|---|---|
| (i) anti-TRBV9 antibody | 5.0-100.0 mg/ml; |
| (ii) histidine buffer being a mixture of | |
| histidine | 0.4-1.0 mg/ml and |
| histidine hydrochloride monohydrate | 0.08-4.2 mg/ml; |
| (iii) osmotic agent being sodium | |
| chloride | 6.0-11.5 mg/ml; |
| (iv) stabilizer being poloxamer 188 or | |
| polysorbate 80 | 0.35-1.3 mg/ml; |
| (v) water for injection | to 1 ml. |

In some embodiments of the invention, the pharmaceutical
composition comprises:

| | |
|---|---|
| (i) anti-TRBV9 antibody; | |
| (ii) histidine buffer being a mixture of | |
| histidine | 0.517 mg/ml |
| | and |
| histidine hydrochloride monohydrate | 0.350 mg/ml; |
| (iii) osmotic agent being sodium | |
| chloride | 9.0 mg/ml; |
| (iv) stabilizer being poloxamer 188 or | |
| polysorbate 80 | 1.0 mg/ml; |
| (v) water for injection | to 1 ml. |

In some embodiments of the invention, the pharmaceutical
composition comprises:

| | |
|---|---|
| (i) anti-TRBV9 antibody | 0.5-300.0 mg/ml; |
| (ii) histidine buffer being a mixture of | 0.517 mg/ml and |
| histidine | 0.350 mg/ml; |
| histidine hydrochloride monohydrate | |
| (iii) osmotic agent being sodium | |
| chloride | 9.0 mg/ml; |
| (iv) stabilizer being poloxamer 188 or | |
| polysorbate 80 | 1.0 mg/ml; |
| (v) water for injection | to 1 ml. |

In some embodiments of the invention, the pharmaceutical
composition comprises:

| | |
|---|---|
| (i) anti-TRBV9 antibody | 25.0 mg/ml; |
| (ii) histidine buffer being a mixture of | 0.517 mg/ml and |
| histidine | 0.350 mg/ml; |
| histidine hydrochloride monohydrate | |
| (iii) osmotic agent being sodium | |
| chloride | 9.0 mg/ml; |
| (iv) stabilizer being poloxamer 188 or | |
| polysorbate 80 | 1.0 mg/ml; |
| (v) water for injection | to 1 ml. |

In one aspect, the present invention relates to a pharmaceutical composition for treating a disease or disorder mediated by T-lymphocytes bearing a TRBV9 segment within the T cell receptor, in a subject in need thereof, comprising anti-TRBV9 antibody and at least one other therapeutically active compound.

In some embodiments, the therapeutically active compound is an antibody, chemotherapeutic agent, or anti-hormonal agent.

In one aspect, the present invention relates to a pharmaceutical composition of anti-TRBV9 antibody, which is provided in dry (i.e. powder or granular) form for reconstitution in a suitable solvent (e.g. water) prior to administration. Such formulation may be prepared by, for example, lyophilisation, i.e. a process which is known in the art as freeze drying, and which involves freezing a product followed by removal of solvent from frozen material.

In one aspect, the present invention relates to a pharmaceutical composition of anti-TRBV9 antibody produced by lyophilization of any of the above pharmaceutical compositions of anti-TRBV9 antibody. Accordingly, the pharmaceutical compositions according to the present invention may be either aqueous pharmaceutical compositions or lyophilized pharmaceutical compositions (lyophilizates).

Lyophilizates are used to produce other dosage forms. For example, a lyophilizate for producing an injectable solution, a lyophilizate for producing a concentrate for producing an injectable solution. Lyophilizates are reconstituted by dissolving same in a suitable solvent, most typically in water for injection. Also, lyophilized compositions are first reconstituted in the required volume of solvent (most typically in water) and then further diluted in a suitable solvent (e.g. 5% glucose solution, 0.9% sodium chloride solution).

The pharmaceutical compositions according to the present invention are typically suitable for parenteral administration as sterile formulations intended for administration in a human body through the breach in skin or mucosal barriers, bypassing the gastrointestinal tract by virtue of injection, infusion and implantation. In particular, it is contemplated that parenteral administration includes, inter alia, subcutaneous, intraperitoneal, intramuscular, intravenous, intraarterial, intrathecal, intraventricular, intraurethral, intracranial, intrasynovial, transdermal injection or infusion, and kidney dialytic infusion techniques. Preferred embodiments include intravenous and subcutaneous routes. Any method for administering peptides or proteins accepted in the art may be suitably employed for the composition of anti-TRBV9 antibody according to the present invention.

In some embodiments of the invention, said pharmaceutical composition of anti-TRBV9 antibody according to the present invention is intended for parenteral administration.

In some embodiments of the invention, said pharmaceutical composition of anti-TRBV9 antibody according to the present invention is intended for intramuscular, intravenous, or subcutaneous administration.

In some embodiments of the invention, said pharmaceutical composition of anti-TRBV9 antibody according to the present invention may be administered intravenously as an infusion.

The pharmaceutical composition of anti-TRBV9 antibody according to the present invention may be used after dilution. To this end, the required volume of the composition is transferred from a vial to an infusion container comprising a sterile 0.9% sodium chloride solution or a sterile 5% dextrose solution. The resulting solution is stirred by gently turning the infusion container over.

Pharmaceutical compositions according to the present invention may be stored in any suitable container. For example, a glass or plastic container, vial, ampoule, syringe, cartridge, or bottle of the desired volume. The containers may be provided with additional means for administration, such as droppers, auto-injectors.

A pharmaceutical composition according to the invention may be manufactured, packaged, or widely sold in the form of a single unit dose or a plurality of single unit doses in the form of a ready formulation. The term “single unit dose” as used herein refers to discrete quantity of a pharmaceutical composition containing a predetermined quantity of an active ingredient. The quantity of an active ingredient typically equals the dose of the active ingredient to be administered in a subject, or a convenient portion of such dose, for example, half or a third of such dose.

In one aspect, the present invention relates to the use of the above pharmaceutical composition of anti-TRBV9 antibody for treating a disease or disorder mediated by T-lymphocytes bearing a TRBV9 segment within the T cell receptor, in a subject in need thereof.

The therapeutically effective amount of pharmaceutical composition of anti-TRBV9 antibody according to the present invention depends on the condition of the subject, the severity of the condition, the previous therapy and the patient's history and response to the therapeutic agent. A suitable dose can be adjusted by the decision of the attending physician so that it can be administered to the patient once or through several injections.

In some embodiments of the invention, the subject of treatment, or patient, is a mammal, preferably a human subject. Said subject may be either male or female, of any age.

In some embodiments of the invention, the disease or disorder mediated by T-lymphocytes bearing a TRBV9 segment within the T cell receptor is selected from the group comprising: arthropathies, inflammatory bowel diseases, eye diseases, vasculitides, circulatory system diseases, kidney diseases, digestive system diseases, lymphoproliferative disorders.

In some embodiments of the invention, the disease or disorder mediated by T-lymphocytes bearing a TRBV9 segment within the T cell receptor is selected from the group comprising:
arthropathies, in particular spondyloarthritides (radiographic axial spondyloarthritis (ankylosing spondylitis), axial spondyloarthritis, peripheral spondyloarthritis, psoriatic arthritis, spondyloarthritis associated with inflammatory bowel diseases, reactive arthritis, undifferentiated peripheral spondyloarthritis), sacroiliitis associated with psoriasis, sacroiliitis associated with inflammatory bowel diseases, undifferentiated oligoarthropathy, juvenile spondylitis/enthesitis-related arthritis, juvenile ankylosing spondylitis (arthritis associated with enthesitis), juvenile arthritis, undifferentiated juvenile arthritis;
inflammatory bowel diseases, in particular ulcerative colitis, Crohn's disease;
eye diseases, in particular non-infectious uveitides, anterior uveitis;
vasculitides, in particular Behcet's disease;
circulatory system diseases, in particular aortitis, fibrosis of aortic and/or mitral valve leaflets with regurgitation, rhythm disturbances, conduction disturbances, left ventricular dysfunction, pericarditis, myocarditis;
kidney diseases, in particular IgA nephropathy;
digestive system diseases, in particular celiac disease;
lymphoproliferative diseases, in particular T cell lymphoma, T cell leukemia.

In some embodiments of the invention, the disease or disorder mediated by T-lymphocytes bearing a TRBV9 segment within the T cell receptor is selected from the group comprising: spondyloarthritides, sacroiliitis associated with psoriasis, sacroiliitis associated with inflammatory bowel diseases, undifferentiated oligoarthropathy, juvenile spondylitis/enthesitis-related arthritis, juvenile ankylosing spondylitis (arthritis associated with enthesitis), juvenile arthritis, undifferentiated juvenile arthritis, ulcerative colitis, Crohn's disease, noninfectious uveitides, anterior uveitis, Behcet's disease, aortitis, fibrosis of aortic and/or mitral valve leaflets with regurgitation, rhythm disturbances, conduction disturbances, left ventricular dysfunction, pericarditis, myocarditis, IgA nephropathy, celiac disease, T cell lymphoma, T cell leukemia.

In some embodiments of the invention, spondyloarthritis is radiographic axial spondyloarthritis (ankylosing spondylitis), axial spondyloarthritis, peripheral spondyloarthritis, psoriatic arthritis, spondyloarthritis associated with inflammatory bowel diseases, reactive arthritis, undifferentiated peripheral spondyloarthritis.

The pharmaceutical compositions may be administered as a single therapeutic agent or in combination with additional therapeutic agents as needed. Thus, in one embodiment, the present methods for treatment and/or prophylaxis are used in combination with administration of a therapeutically effective amount of another active agent. The other active agent may be administered before, during or following the administration of the pharmaceutical compositions according to the present invention. The other active agent may be administered as part of the present composition or, alternatively, as a separate formulation.

### Implementation of the Invention

The following examples are provided for better understanding of the invention. These examples are for purposes of illustration only and are not to be construed as limiting the scope of the invention in any manner.

All publications, patents, and patent applications cited in this specification are incorporated herein by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended embodiments.

### Methods

### 1. Preparation of Samples of Monoclonal Antibody to TRBV9.

Antibody samples at a concentration of 5-50 mg/ml were prepared in Stirred Cell (Millipore) under pressure. To this end, the initial antibody formulation was placed in a cell, the protein was concentrated under a compressed air stream to a desired concentration under continuous stirring, following which at least 10-fold volume of an aqueous solution with the target formulation comprising buffering agents, osmotic agents and, if necessary, additional water soluble stabilizers was stepwise added to the cell. After diafiltration, we continued concentrating to a concentration exceeding the target one, unloaded it from the cell, and the exact protein concentration was measured by UV spectroscopy. Then we added a concentrate of the corresponding solution of excipients and a concentrate of surfactant to the sample to produce a solution with the target protein concentration.

Protein samples at a concentration of 20 mg/ml or greater were prepared in Pellicon cassettes (Millipore) in a tangential flow mode. To this end, the initial antibody formulation was placed in a diafiltration tank, the protein was concentrated to a desired concentration, at least 10-fold volume of the solution with the target formulation comprising buffering agents, and, if necessary, additional water soluble stabilizers was then supplied to the system. Following diafiltration, we continued concentrating to a concentration exceeding the target one, unloaded it from the system, and the exact protein concentration was measured.

When obtaining formulations comprising solubilizers, e.g. poloxamer 188, the surfactant concentrates were added to the antibody following diafiltering and concentrating, with the final dilution of the antibody to the target concentration with a solution of excipients.

During aseptic filling into the final container (for example, a sterile glass/plastic vessel, vial or syringe), the antibody solution was filtered using a 0.22 μm sterilizing membrane.

### 2. Determination of Protein Concentration in Test Samples

Protein concentration was determined by UV spectrophotometry at a wavelength of 280 nm in UV spectrophotometry plates. Each sample was diluted with the appropriate solution of excipients to a concentration of about 0.5 mg/ml. 150 μl of the diluted sample was placed into a well of UV spectrophotometry plate. Optical density of solutions in the plate wells was measured using a plate spectrophotometer at a wavelength of 280 nm. An appropriate solution of excipients was used as a reference solution.

Concentration (mg/ml) of protein (C) was calculated using the following formula:

$$C = \frac{( {A ( 280 )}_{prot} - {A ( 280 )}_{plac} ) * b}{ε * l} ,$$

where A280 is the value of optical density at a wavelength of 280 nm;
ε is the extinction coefficient of test protein;
b is the total dilution factor for a sample;
l is the layer thickness in a plate well. cm. where it is 0.56 cm for a standard 200 μl 96-well plate. It is 1 cm for a 175 μl half-area plate. It is 0.42 cm for a 150 μl full-size plate.

### 3. Determination of Protein Aggregation Temperature by Dynamic Light Scattering.

The point of aggregation of test proteins (at a concentration of 1 to 5 mg/ml) was determined using DynaPro Plate Reader II. To this end, 35 μl of the solution was placed into a well of a black polymer plate with an optically clear bottom, which was gradually heated in the instrument while constantly measuring scattered light intensity.

### Measurement Settings:

Initial measurement temperature—25° C.
Scattered light intensity at θ=158°.
Number of measurements per replicate—3.
Time per measurement—5 s.
Heating rate—0.15° C./min.
Final temperature—80° C.

The temperature trend and aggregation point were determined using the Dynamics V7 software.

### 4. Determination of Protein Melting Point by Differential Scanning Fluorimetry.

Sypro Orange fluorescent stain was added to the protein sample. The sample was analyzed in a CFX96 C1000 Touch Thermal Cycler amplifier in real time mode. Heating was from 25 to 85° C., the detection channel was ROX. CFX Manager (Bio-Rad) software was used to process the results.

### 5. Determination of Hydrodynamic Radius of Particles in Solution by Dynamic Light Scattering

For analysis, 35 μl of sample at each concentration was placed into wells of a black polymer plate with an optically clear bottom. The analysis was performed using the DynaPro Plate Reader II instrument. Each well was analyzed 10 times. The resulting data was processed in the Dynamics V7 software.

### 6. Determination of Diffusion Interaction Parameter (k_{D}) by Dynamic Light Scattering

A number of protein solutions from 30 mg/ml to 0.94 mg/ml were produced by stepwise dilution. Appropriate solutions of excipients were used as a solvent.

For analysis, 35 μl of sample at each concentration was placed into wells of a black polymer plate with an optically clear bottom. The analysis was performed using the DynaPro Plate Reader II instrument. Each well was analyzed 10 times. The resulting data was processed in the Dynamics V7 software, where dependence of the diffusion coefficient on protein concentration in solution was plotted and the inclination of line for the resulting dependence was determined.

### 7. Determination of Thermal Stability Under 50° C. Thermal Stress (TS50).

Test samples were divided into 2 aliquots of 150 μl each and placed into separate glass vials: 1 vial per composition was stored in a refrigerator at 5±3° C., the rest vials were placed in a thermostat and incubated at 50° C. for 96 hours or 120 hours. When selecting control points or following heating, the vials were removed from the thermostat, kept at room temperature for about 15 minutes, and transferred for analysis.

### 8. Determination of Colloidal Stability Under Shaking (SH800).

Test samples were divided into 2 aliquots of 150 μl each and placed into glass vials, 1 vial per formulation was stored in a refrigerator at 5±3° C., the rest vials were placed into a thermal shaker and shaken at 800 rpm at 5±3° C. for 96 or 120 hours. During the selection of control points or following stress, the vials were removed from the thermal shaker and transferred for analysis.

### 9. Determination of Colloidal Stability Under Freeze-Thaw (FT(-20)).

Test samples were divided into 2 aliquots and placed into plastic vials: 1 vial per formulation was stored in a refrigerator at 5±3° C., the rest vials were stored in a freezer at minus (20 t 2°) C until completely frozen. Thereafter, the vials were removed from the freezer, kept at room temperature until the contents were completely thawed; the solutions were mixed using a vortex and placed back into the freezer. This was repeated the required number of times. Following stress, the vials were removed from the freezer, kept at room temperature until the contents were completely thawed; the solutions were mixed using a vortex and transferred for analysis.

### 10. Determination of Stability Under Acid Hydrolysis (Acid).

Test samples were divided into 2 aliquots and placed into polymer vials: 1 vial per formulation was stored in a refrigerator at 5±3° C. (input control may be transferred for analysis once for all studies at the start of storage), pH for the rest vials was adjusted to 4.0±0.1 or 3.0±0.1 with hydrochloric acid solution while stirring, thereafter, they were transferred to a refrigerator at 5±3° C. for storage. After 1 hour or 24 hours, hydrolysis was quenched while stirring by adding sodium hydroxide solution to the initial pH value. The solutions were then transferred for analysis.

### 11. Determination of Stability Under Basic Hydrolysis (Basic)

Test samples were divided into two aliquots and placed into polymer vials: one vial per formulation was stored in a refrigerator at 5±3° C. (input control may be transferred for analysis once for all studies at the start of storage), pH for the rest vials was adjusted to 9.0±0.1 with sodium hydroxide solution while stirring, thereafter, they were transferred to a refrigerator for storage at 5±3° C. After 1 hour or 24 hours, hydrolysis was quenched while stirring by adding hydrochloric acid solution to the initial pH value. The solutions were then transferred for analysis.

### 12. Accelerated Storage.

The test samples were divided into separate aliquots (one aliquot for the input control—it is allowed to transfer for analysis once for all studies at the start of storage) and placed in separate sterile glass vials: part of the vials for each formulation was placed in the refrigerator for storage at 5±3° C. (input control), the rest vials were placed in a thermostat and incubated at 25±2° C. for 6 months, periodically selecting control points according to the plan. When selecting control points and following storage, the vials were removed from the thermostat and transferred for analysis.

### 13. Determination of Sample Purity by Size-Exclusion High-Performance Liquid Chromatography (SE HPLC).

Column: Tosoh TSK-Gel G3000SWXL 7.8 mm ID×30 cm, 5 μm.
Pre-column: TSK-Gel Guard SW_{XL}, 6.0 mm ID×4.0 cm, 7 μm, 300 Å.
Column temperature: 25° C.
Mobile phase flow rate: 0.5 ml/min.
Injection volume: 25 μl.
Sample concentration: 0.5 mg/ml.
Detector wavelength: 214 and 360 nm.
Elution time: 30 min.
Mobile phase: Disodium hydrogen phosphate anhydrous 14.196 mg/ml.
   Sodium chloride 11.688 mg/ml.

The mobile phase pH was adjusted to 6.9 with orthophosphoric acid.

### 14. Evaluation of Charge Variant Profile in Capillary on the Caliper LabChip GX II Instrument

Analysis was conducted in accordance with the instructions for the HT Protein charge variant kit. Test samples were adjusted to a protein concentration of 1 mg/ml by diluting or concentrating in 0.5 ml Amicon Ultra 10 kDa centrifuge filters (Millipore) (depending on the initial concentration of samples). Protein content was followed by UV spectrophotometry at a wavelength of 280 nm.

2 μl of carboxypeptidase solution was added to each resulting sample, and the samples were incubated for 2 hours at a temperature of 37±2° C. After the specified time, the samples were dialyzed against water in Amicon Ultra centrifuge tubes and concentrated to 2 mg/ml.

A 96-well plate was loaded with amounts, as specified in the instructions, of Labelling Buffer solution, Dye Mixture solution and 25 μl of the test sample, the plate was placed in a dark place for 10 minutes, each well was then loaded with 60 μl of water and mixed.

The plate with solutions was centrifuged using a plate centrifuge rotor and placed in the Caliper LabChip GX II instrument. The analysis used a special chip which was filled with Running Buffer solution with pH in accordance with the instructions. The results were processed using LabChip GX software.

### 15. Determination of Charge Variant Profile by Ion Exchange High Performance Liquid Chromatography (IE HPLC).

Column: ProPac WCX-10, 4×250 mm, particle size: 10 μm
Pre-column: ProPac WCX-10G, 4×50 mm, particle size: 10 μm
Eluent A: Solution of 28.8 mM sodium dihydrogen phosphate, pH=6.5
Eluent B: Solution of 28.8 mM sodium dihydrogen phosphate, 250 mM sodium chloride, pH=6.5
Flow rate: 0.7 ml/min.
Column temperature: 30° C.
Autosampler temperature: 4° C.
Detector: UV, 280 nm, bandwidth: 16 nm
Reference wavelength: 360 nm, 100 nm bandwidth
Sample volume: 40 μl, Needle wash mode
Chromatography time: 60 min.

The test sample was diluted to a concentration of 1.0 mg/ml and treated with carboxypeptidase B in a ratio of 100:2.8; the resulting solution was stirred and incubated for 2 hours at a temperature of 37±2° C.

### Elution Mode:

| Item | Time, | Eluent | Eluent | |
|---|---|---|---|---|
| no. | min | A, % | B, % | Elution mode |
| 1 | 0-3 | 91 | 9 | Isocratic |
| 2 | 3-44 | 91 → 70 | 9 → 30 | Linear gradient |
| 3 | 44-45 | 70 → 0 | 30 → 100 | Linear gradient |
| 4 | 45-51 | 0 | 100 | Isocratic |
| 5 | 51-52 | 0 → 91 | 100 → 9 | Linear gradient |
| 6 | 52-60 | 91 | 9 | Isocratic |

16. Determination of Purity and Related Impurities by Capillary Gel Electrophoresis in the Presence of Sodium Dodecyl Sulfate (CE Red. And Non-Red.).

The sample was diluted to a concentration of 4.0 mg/ml. 25 μl of the resulting solution was placed in a 1.5 ml microtube; 70 μl of SDS-MW Sample Buffer, 2 μl of internal standard having a molecular weight of 10 kDa, 5 μl of 0.5M iodoacetamide solution (CE non-red.) or 5 μl of 2-mercaptoethanol (CE red.) were added thereto. The resulting solution was stirred for 15 s, centrifuged for 5 s at 2800 rpm and placed in a solid state thermostat at 65° C. for 4 min (CE non-red.) or at 70° C. for 10 min (CE red.). The solution was cooled to room temperature.

The SDS MW Separation—PA 800 plus.met analysis method was used in 32 Karat Software.

### Conditions of Capillary Gel Electrophoresis:

Capillary: 50 μm×30.2 cm
Effective length of capillary: 20.0 cm
Polarity: reverse, inlet on left side (−), outlet on right side (+)
Capillary temperature: 25° C.
Analysis time and separation voltage: 35 min, 15 kV
Detection wavelength: 220 nm.

### 17. Determination of Purity of Samples on the Caliper Labchip GXII Instrument Under Non-Reducing Conditions.

### Preparation of Samples for Analysis.

700 μl of HT Protein Express Sample Buffer was used to prepare a reconstitution solution. Alkylating agent—24.5 μl of 1M iodoacetamide (IAM)—was added to the buffer.

35 μl of reconstitution buffer was added to the microtube. Samples were diluted to a concentration of 2 mg/ml. 5 μl of sample were added to the tube. Samples were denatured at 100° C. for 5 minutes. Tubes were mixed using a vortex, thereafter 70 μl of water was added, and the tubes were mixed using a vortex. 44 μl of each sample was transferred to the wells of 96-well plate.

Working solutions and chip preparation were according to the manufacturer's protocol using HT Protein Express Reagent Kit. Launch of the analysis is a standard procedure. Method of analysis: HT Protein Express 200.

### 18. Determination of Acid-Base Profile of Samples by Capillary Zone Electrophoresis

Test sample having a volume of 50-100 μl was placed in the centrifuge ultrafilter of the Amicon Ultra type (0.5 ml, 10 kDa, UFC501096, Millipore Ltd.), 400 μl of purified water was added, and ultrafiltration was performed using a centrifuge for 10 min at 10000 rpm at a temperature of 10° C. Ultrafiltration procedure was performed 3 times, each time 400-450 μl of water was added to each residue in centrifuge ultrafilters. Following ultrafiltration, the residues were transferred into a 0.5 ml microtube.

Protein content in the resulting solution was determined by spectrophotometry.

We launched 32 Karat Software and chose the instrument with UV or PDA detectors. A filter with a wavelength of 214 nm was selected for the UV detector.

### Analysis Conditions:

Separation voltage: 30 kV normal (inlet on left side
Polarity: (+), outlet on right side (−))
Capillary temperature in cartridge: 25° C.
Temperature in the sample tray: 10° C.
Detection wavelength: 214 nm
Data transfer rate: 4 Hz
Analysis time: 15 min

The quartz capillary with an inner diameter of 50 μm and a length of 30.2 cm (effective length was 20 cm) was prepared. The capillary was installed in a cartridge with an aperture of 200×100 μm. The following solutions were introduced into plastic vials with a volume of 1.5 ml:
1.5 ml of 0.1M HCl solution;
1.5 ml of separation buffer;
1.5 ml of purified water;
1.0 ml of purified drain water.

The “CZE Conditioning—PA 800 plus ABP.met” analysis method was chosen in 32 Karat Software.

### 19. Measurement of Viscosity of Samples by Viscometry

Test sample in a volume of 350-400 μl was taken with a pipette for viscosity measurement microVISC Pipettes. The pipette was placed in the RheoSence microVISC viscometer. Viscosity was measured using a viscometer thermostat, in automatic mode (AUTO) at 25.0° C.

### 20. pH Measurement.

Test sample in the volume of 200-300 μl was transferred into a microtube, pH was measured on the Mettler Toledo SevenEasy instrument using Mettler Toledo InLabUltraMicro electrode.

### 21. Determination of Stability Under Oxidation.

Test samples were divided into two aliquots of 150 μl each and placed into separate glass vials: one vial per formulation was stored in a refrigerator at 5±3° C., hydrogen peroxide was added to the rest of the samples to final concentration of hydrogen peroxide of 0.1% in samples, the samples were aged for 4 hours at (5±3) ° C. Oxidation was quenched by adding an equivalent amount of L-methionine.

### 22. Processing of Results.

The absolute change in quality indicators when under stresses was calculated by the formula:

$$Δ = ( value following stress - value before stress )$$

Absolute change in the charge variant profile was calculated by the formula:

$$= \vert acidic fraction content before stress - acidic fraction content following stress \vert + \vert basic fraction content before stress - basic fraction content following stress \vert + \vert dominating fraction content before stress - dominating fraction content following stress \vert$$

### 23. Preparation of Infusion Solutions.

Solutions were prepared by diluting the test formulations with 0.9% NaCl solution to protein concentration of 0.5 mg/ml.

### EXAMPLES

The examples below are given for anti-TRBV9 antibody comprising a heavy chain variable domain comprising:
(a) HCDR1 having the amino acid sequence of SEQ ID NO: 1,
(b) HCDR2 having the amino acid sequence of SEQ ID NO: 2, and
(c) HCDR3 having an amino acid sequence selected from the group consisting of SEQ ID NO: 3, SEQ ID NO: 4, SEQ ID NO: 5 or SEQ ID NO: 6;

1) a light chain variable domain comprising:
(a) LCDR1 having the amino acid sequence of SEQ ID NO: 7,
(b) LCDR2 having the amino acid sequence of SEQ ID NO: 8, and
(c) LCDR3 having the amino acid sequence of SEQ ID NO: 9.

In particular, the examples below are given for anti-TRBV9 antibody comprising a heavy chain variable domain having the amino acid sequence of SEQ ID No: 14 and a light chain variable domain having the amino acid sequence of SEQ ID No: 17.

In particular, the examples below are given for anti-TRBV9 antibody comprising a heavy chain having the amino acid sequence of SEQ ID No: 22 and a light chain having the amino acid sequence of SEQ ID No: 25 (candidate 42 or antibody 42).

### Example 1. Selection of Nature of Buffer System

Buffering agents selected to be used in the present study are excipients suitable for pharmaceutical use and used in therapeutic protein-based drug formulations. For the basis of the pharmaceutical composition, 4 typical buffer systems suitable for parenteral administration were selected: acetate, citrate, histidine and phosphate buffer systems. To facilitate comparison of the stabilizing properties of buffer solutions of different nature, pH and concentration of solutions were reduced to 5.5 and 20 mM, respectively.

To assess the suitability of buffer systems, we studied the effect of buffer solution's nature on colloidal and conformational stabilities of protein. To assess the effect, we determined the aggregation temperature, melting point, diffusion interaction parameter, concentration ability, change in purity and acid-base profile following thermal stress.

Table 1 shows the formulations of the test buffer solutions.

TABLE 1
Test formulations.

| No. | Designation | Formulation | |
|---|---|---|---|
| 1 | 20 mM Acet | anti-TRBV9 | 1.5-* mg/ml |
| | pH 5.5 | Sodium acetate trihydrate | 2.311 mg/ml |
| | | Acetic acid glacial | to pH 5.5 |
| | | Water for injection | to 1 ml |
| 2 | 20 mM Cit | anti-TRBV9 | 1.5-* mg/ml |
| | pH 5.5 | Sodium citrate dihydrate | 4.465 mg/ml |
| | | Anhydrous citric acid | 0.925 mg/ml |
| | | Water for injection | to 1 ml |
| 3 | 20 mM His | anti-TRBV9 | 1.5-* mg/ml |
| | pH 5.5 | L-histidine | 0.746 mg/ml |
| | | L-histidine hydrochloride | 3.185 mg/ml |
| | | monohydrate | |
| | | Water for injection | to 1 ml |
| 4 | 20 mM PB | anti-TRBV9 | 1.5-* mg/ml |
| | pH 5.5 | Sodium dihydrogen | 2.97 mg/ml |
| | | phosphate dihydrate | 0.172 mg/ml |
| | | Disodium hydrogen | |
| | | phosphate dihydrate | |
| | | Water for injection | to 1 ml |

*max protein concentration is limited by direct concentrating and is specified in Table 2.

### Study of Stability Prediction Indicators.

The diffusion interaction parameter (k_{D}) reflects sample's diffusion coefficient as a function of concentration of molecules. If the diffusion coefficient is decreased with increasing concentration (k_{D}<0), then polydispersity of the given solution is increased and larger particles are formed therein. Such samples have low solubility and tend to aggregate, and formulations thereof are not recommended for use.

The aggregation temperature and melting point make it possible to assess the protein's tendency to aggregation. The most stable samples are those in which particle aggregation begins at a higher temperature and where smaller particles are formed under heating.

Results of the study of aggregation temperature by method 3, melting point by method 4, diffusion interaction parameter by method 6 and viscosity by method 19 are shown in Table 2.

TABLE 2
Results of study of stability and direct concentrating of anti-TRBV9 in various formulations.

| | Test formulation, | | | | | | | | | | | | |
|---|---|---|---|---|---|---|---|---|---|---|---|---|---|
| | mg/ml | | | | | | | | | | | | |
| | | For- | | | | | | | | | | | |
| | | mulation | DSF | DLS | | Direct concentrating | | | | | | | |
| | | desig- | Tm, | Tag, | | C in, | V in, | C fin., | V fin., | Yield, | Cfin./ | C η, | η, |
| No. | Protein | nation | ° C. | ° C. | kd | mg/ml | ml | mg/ml | ml | % | Cin. | mg/ml* | cP |
| 1 | 5** | Acet | 65.20 | 63.02 | 6.03E−03 | 1 | 1000 | 186 | 130 | 24.18 | 186 | 160 | 10.30 |
| 2 | 5** | PB | 65.30 | 63.62 | −1.00E−03 | 1 | 1000 | 187 | 100 | 18.70 | 187 | 160 | 17.15 |
| 3 | 5** | His | 63.50 | 63.03 | 7.12E−03 | 1 | 1000 | 212 | 85 | 18.02 | 212 | 160 | 11.57 |
| 4 | 5** | Cit | 64.70 | 58.96 | −8.27E−03 | 1 | 1000 | 165 | 138 | 22.77 | 165 | 160 | 16.89 |

*Concentration of a sample for which viscosity was determined.
**Protein concentration is method-specific and given in the method description.

Determination of thermal Stability.

Thermal stability was assessed by method 7. Before and following thermal stress, purity was determined by SE HPLC by method 13, purity was determined by electrophoresis in non-reducing conditions according to method 17, charge variant profile was determined in Labchip by method 14, hydrodynamic radius was determined by method 5. Absolute change in charge variant profile was calculated by method 22. The results are shown in Table 3.

TABLE 3
Results of study of stability of anti-TRBV9 in various formulations under thermal stress.

| | Test formulation, | | | | | | | Labchip | | | | | | | | |
|---|---|---|---|---|---|---|---|---|---|---|---|---|---|---|---|---|
| | mg/ml | | Hydro- | SE HPLC | | | | non-red. | | Labchip charge variant profile, % | | | | | | |
| | | For- | dynamic | Aggregates, % | | Monomer, % | | Monomer, % | | | | | | | | Δ |
| | | mulation | radius, | | Δ | | Δ | | Δ | | | | | | | abs. |
| | | desig- | nm | | TS50 | | TS50 | | TS50 | Alc. | Base | Acid. | Alc. | Base | Acid. | TS50 |
| No. | Protein | nation | IC | IC | 96H | IC | 96H | IC | 96H | IC | | | Δ TS50 96H | | | 96H |
| 1 | 5 | Acet | 5.4 | 0.760 | 0.600 | 98.420 | −1.108 | 93.0 | n/a | 6.7 | 63.1 | 30.3 | 15.9 | −14.4 | −1.5 | 31.8 |
| 2 | 5 | PB | 5.8 | 0.753 | 0.869 | 98.439 | −1.613 | 93.3 | −0.8 | 6.4 | 62.2 | 31.4 | 15.7 | −14.8 | −0.9 | 31.4 |
| 3 | 5 | His | 5.4 | 0.688 | 0.937 | 98.535 | −1.687 | 92.5 | −1.9 | 6.8 | 61.7 | 31.6 | 26.9 | −18.8 | −8.1 | 53.7 |
| 4 | 5 | Cit | 7.5 | 0.740 | 1.016 | 98.477 | −1.628 | 93.1 | −6.0 | 6.4 | 62.5 | 31.1 | 11.9 | −13.4 | 1.5 | 26.9 |

* Protein concentration is method-specific and given in the method description.

Acetate buffer-based formulations showed excellent stabilizing properties in terms of conformational stability (high melting point), colloidal stability (high aggregation temperature by DLS, k_{D}>0, high protein concentration following direct concentrating, low viscosity of solution at protein concentration of 160 mg/ml, low aggregate content following thermal stress followed by SE HPLC), chemical stability (satisfactory values for the change in the isoform profile following thermal stress).

The histidine buffer-based formulations showed excellent colloidal stability: k_{D}>0, high value of resulting concentration under direct concentrating, low solution viscosity at protein concentration of 160 mg/ml.

### Example 2. Selection of pH/Concentration of Buffer Solution

In this study, 2 typical buffer systems, acetate and histidine buffer systems, that are suitable for parenteral administration were selected as the basis of the pharmaceutical composition. Study was conducted in full two-factor experiment design with two levels and a center point. The pH level (from 5.0 to 6.0 for the acetate buffer solution and from 5.5 to 6.5 for the histidine buffer solution) and the concentration of buffer agents (from 5 to 50 mM) were studied as quantitative factors.

To assess the suitability of buffer systems, we studied the effect of buffer solution's nature on colloidal and conformational stabilities of protein. The aggregation temperature, melting point, diffusion interaction parameter, changes in purity and acid-base profile following thermal stress, and the ability to concentrate were determined as a response. The test formulations are shown in Table 4.

TABLE 4
Test formulations.

| | | | | |
|---|---|---|---|---|
| | | Con- | | |
| | | centration, | | |
| No. | pH | mM | Designation | Formulation |

Acetate buffer solution

| | | | | | |
|---|---|---|---|---|---|
| 1 | 6.0 | 5 | pH max, C | anti-TRBV9 | 1.5-30 mg/ml |
| | | | min | Sodium acetate | 0.644 mg/ml |
| | | | | trihydrate | |
| | | | | Acetic acid | to pH 6.0 |
| | | | | glacial | |
| | | | | Water for injection | to 1 ml |
| 2 | 6.0 | 50 | pH max, C | anti-TRBV9 | 1.5-30 mg/ml |
| | | | max | Sodium acetate | 6.443 mg/ml |
| | | | | trihydrate | |
| | | | | Acetic acid | to pH 6.0 |
| | | | | glacial | |
| | | | | Water for injection | to 1 ml |
| 3 | 5.0 | 50 | pH min, C | anti-TRBV9 | 1.5-30 mg/ml |
| | | | max | Sodium acetate | 4.355 mg/ml |
| | | | | trihydrate | |
| | | | | Acetic acid | to pH 5.0 |
| | | | | glacial | |
| | | | | Water for injection | to 1 ml |
| 4 | 5.0 | 5 | pH min, C | anti-TRBV9 | 1.5-30 mg/ml |
| | | | min | Sodium acetate | 0.436 mg/ml |
| | | | | trihydrate | |
| | | | | Acetic acid | to pH 5.0 |
| | | | | glacial | |
| | | | | Water for injection | to 1 ml |
| 5, | 5.5 | 27.5 | C mid, pH | anti-TRBV9 | 1.5-30 mg/ml |
| 5' * | | | mid | Sodium acetate | 2.889 mg/ml |
| | | | | trihydrate | |
| | | | | Acetic acid | to pH 5.5 |
| | | | | glacial | |
| | | | | Water for injection | to 1 ml |

Histidine buffer solution

| | | | | | |
|---|---|---|---|---|---|
| 6 | 6.5 | 5 | pH max, C | anti-TRBV9 | 1.5-30 mg/ml |
| | | | min | L-histidine | 0.58 mg/ml |
| | | | | L-histidine h/c | 0.27 mg/ml |
| | | | | Monohydrate | |
| | | | | Water for injection | to 1 ml |
| 7 | 6.5 | 50 | pH max, C | anti-TRBV9 | 1.5-30 mg/ml |
| | | | max | L-histidine | 5.8 mg/ml |
| | | | | L-histidine h/c | 2.7 mg/ml |
| | | | | monohydrate | |
| | | | | Water for injection | to 1 ml |
| 8 | 5.5 | 50 | pH min, C | anti-TRBV9 | 1.5-30 mg/ml |
| | | | max | L-histidine | 2.3 mg/ml |
| | | | | L-histidine h/c | 7.4 mg/ml |
| | | | | monohydrate | |
| | | | | Water for injection | to 1 ml |
| 9 | 5.5 | 5 | pH min, C | anti-TRBV9 | 1.5-30 mg/ml |
| | | | min | L-histidine | 0.23 mg/ml |
| | | | | L-histidine h/c | 0.74 mg/ml |
| | | | | monohydrate | |
| | | | | Water for injection | to 1 ml |
| 10, | 6.0 | 27.5 | pH mid, C | anti-TRBV9 | 1.5-30 mg/ml |
| 10' * | | | mid | L-histidine | 2.25 mg/ml |
| | | | | L-histidine h/c | 2.25 mg/ml |
| | | | | monohydrate | |
| | | | | Water for injection | to 1 ml |

* 2 replicates per formulation

### Study of Stability Prediction Indicators.

Results of determination of aggregation temperature by method 3, melting point by method 4, diffusion interaction parameter by method 6 and ability to concentrate by method 1 are shown in Table 5.

TABLE 5
Results of study of stability and direct concentrating of anti-TRBV9 in various formulations.

| | Test formulation, mg/ml | | | | | |
|---|---|---|---|---|---|---|
| | | Formulation | | | | Direct concentrating |
| No. | Protein | designation | Tm, ° C. | Tag, ° C. | kd | C fin., mg/ml |
| 1 | 5* | Acet, 6.0, 5 mM | 65.4 | 64.32 | 3.82E−02 | 36.8 |
| 2 | 5* | Acet, 6.0, 50 mM | 65 | 62.28 | −2.45E−03 | 37.4 |
| 3 | 5* | Acet, 5.0, 50 mM | 63.3 | 62.21 | 7.13E−03 | 38.18 |
| 4 | 5* | Acet, 5.0, 5 mM | 65.5 | 67.15 | 7.75E−02 | 40.42 |
| 5 | 5* | Acet, 5.5, 27.5 mM | 65 | 63.99 | 2.92E−03 | 38.17 |
| 5′ | 5* | Acet, 5.5, 27.5 mM | 65 | n/a | n/a | 38.17 |
| 6 | 5* | His, 6.5, 5 mM | 65.2 | 66.04 | 6.12E−02 | 41.4 |
| 7 | 5* | His, 6.5, 50 mM | 65.4 | 63.57 | −3.66E−04 | 37.3 |
| 8 | 5* | His, 5.5, 50 mM | 62.1 | 61.7 | 7.00E−03 | 32.7 |
| 9 | 5* | His, 5.5, 5 mM | 64.1 | 66.73 | 5.84E−02 | 34.54 |
| 10 | 5* | His, 6.0, 27.5 mM | 65.2 | 62.81 | 7.37E−03 | 35.4 |
| 10′ | 5* | His, 6.0, 27.5 mM | 65.2 | n/a | n/a | 35.4 |

*Protein concentration is method-specific and given in the method description.
Formulations 5′ and 10′ are repeat formulations 5 and 10, respectively.

### Determination of Thermal Stability.

M Thermal stability was assessed by method 7. Before and following thermal stress, purity was determined by SE HPLC by method 13, purity was determined by electrophoresis in non-reducing conditions by method 17, charge variant profile was determined in capillary by method 14, hydrodynamic radius was determined by method 5, absolute change in charge variant profile was determined by method 22. The results are shown in Table 6.

TABLE 6
Results of study of stability of anti-TRBV9 in various formulations under thermal stress.

| | | | | | | | | | Labchip | | | | | | | | |
|---|---|---|---|---|---|---|---|---|---|---|---|---|---|---|---|---|---|
| | | | Hydrodynamic | | SE HPLC | | | | non-red. | | Labchip acid-base profile, % | | | | | | |
| | Test formulation, | | radius, nm | | Aggregates, % | | Monomer, % | | Monomer, % | | | | | | | | Δ |
| | mg/ml | | | Δ | | Δ | | 4 | | Δ | | | | | | | abs. |
| | | Formulation | | TS50 | | TS50 | | TS50 | | TS50 | Alc. | Base | Acid. | Alc. | Base | Acid. | TS50 |
| No. | Protein | designation | IC | 96H | IC | 96H | IC | 96H | IC | 96H | IC | | | Δ TS50 96H | | | 96H |
| 1 | 5* | Acet, 6.0, 5 mM | 4.6 | 0 | 0.74 | 0.67 | 98.08 | −1.46 | 94.6 | −2.3 | 7.95 | 66.46 | 25.6 | 16.3 | −16.8 | 0.5 | 33.6 |
| 2 | 5* | Acet, 6.0, | 5.6 | 0.7 | 0.76 | 1.5 | 98.12 | −2.52 | 94.5 | −2.3 | 8.44 | 66.65 | 24.92 | 13.8 | −15.1 | 1.3 | 30.3 |
| | | 50 mM | | | | | | | | | | | | | | | |
| 3 | 5* | Acet, 5.0, | 5.4 | 0.5 | 0.68 | 2.41 | 98.15 | −3.74 | 94.1 | −1.2 | 9.3 | 65.7 | 25.01 | 30.5 | −24.3 | −6.2 | 61 |
| | | 50 mM | | | | | | | | | | | | | | | |
| 4 | 5* | Acet, 5.0, 5 mM | 3.9 | −0.3 | 0.69 | 0.08 | 98.2 | −1.07 | 93.9 | −3.4 | 9.28 | 66.06 | 24.68 | 27.7 | −23.0 | −4.7 | 55.3 |
| 5 | 5* | Acet, 5.5, | 5.5 | 0.2 | 0.74 | 1.35 | 98.16 | −2.27 | 91.2 | −1.1 | 8.99 | 66.62 | 24.4 | 20.5 | −18.9 | −1.6 | 41.1 |
| | | 27.5 mM | | | | | | | | | | | | | | | |
| 5′ | 5* | Acet, 5.5, | 5.5 | 0.2 | 0.76 | 1.33 | 98.13 | −2.22 | 90.9 | −1 | 8.91 | 66.88 | 24.22 | 20.7 | −19.4 | −1.3 | 41.4 |
| | | 27.5 mM | | | | | | | | | | | | | | | |
| 6 | 5* | His, 6.5, 5 mM | 3.8 | −0.3 | 0.81 | 0.21 | 98.21 | −0.97 | 91 | −0.1 | 8.12 | 66.84 | 25.04 | 12.0 | −14.4 | 2.4 | 28.8 |
| 7 | 5* | His, 6.5, 50 mM | 5.5 | 0.4 | 0.71 | 0.83 | 98.13 | −1.82 | 90.8 | −0.9 | 8.08 | 67.55 | 24.37 | 12.9 | −15.2 | 2.4 | 30.5 |
| 8 | 5* | His, 5.5, 50 mM | 5.5 | 0.8 | 0.65 | 2.27 | 98.24 | −3.33 | 89 | −0.1 | 9.19 | 66.59 | 24.22 | 33.8 | −25.6 | −8.2 | 67.7 |
| 9 | 5* | His, 5.5, 5MM | 4.3 | −0.2 | 0.7 | 0.05 | 98.24 | −0.85 | 91 | −0.5 | 8.72 | 66.58 | 24.71 | 29.4 | −23.1 | −6.3 | 58.7 |
| 10 | 5* | His, 6.0, | 5.5 | 0 | 0.72 | 0.71 | 98.16 | −1.51 | 90.8 | 2.1 | 8.33 | 66.83 | 24.86 | 19.8 | −17.9 | −2.0 | 39.6 |
| | | 27.5 mM | | | | | | | | | | | | | | | |
| 10′ | 5* | His, 6.0, | 5.5 | 0.1 | 0.73 | 0.69 | 98.13 | −1.43 | 92.7 | 0.3 | 8.31 | 67.37 | 24.32 | 19.3 | −18.8 | −0.6 | 38.6 |
| | | 27.5 mM | | | | | | | | | | | | | | | |

*Protein concentration is method-specific and given in the method description.
Formulations 5′ and 10′ are repeat formulations 5 and 10, respectively.

Acetate buffer-based pharmaceutical compositions showed excellent conformational stability, high melting point. Further, these formulations are characterized by the following: high aggregation temperature and k_{D} value, a slight increase in impurities (followed by SE HPLC and LabChip) and no changes in the hydrodynamic radius under thermal stress. Further, these formulations showed the ability to stabilize the charge variant profile.

Histidine buffer-based formulations demonstrated excellent conformational stability: the test protein in the given buffer has a high melting point, colloidal stability: high aggregation temperature and k_{D} value, which indicates its increased stability and less tendency to aggregation under concentrating and diafiltration. These formulations showed high concentration values under direct concentrating and, according to the control results, there is a slight change in the quality of protein under thermal stress (when followed by SE HPLC and Labchip in non-reducing conditions). Also, these formulations demonstrate excellent stabilization of the acid-base profile.

### Example 3. Selection of Osmotic Agent

The present study employs histidine buffer system as the basis of the pharmaceutical composition. Excipients suitable for parenteral administration were studied to be used as osmotic agents. Amount of osmotic agents which provides osmolarity that is close to organism's physiological media was calculated by the formula:

$${C}_{osm} = \frac{m}{M} \cdot n \cdot 1000 ,$$

where Cₒₛₘ is the solution osmolarity, miliosmole per liter (mOsm/l);
m is the content of substance in solution, g/l;
M is the molar mass of the substance, g;
n is the total number of ions produced from one solute molecule as a result of dissociation (n=1 for non-dissociating substances; n=2, 3 for substances that produce respective number of ions upon dissolution).

To assess the suitability of buffer systems, we studied the effect of buffer solution's nature on colloidal and conformational stabilities of protein. The aggregation temperature, melting point, diffusion interaction parameter, changes in purity and acid-base profile following thermal stress, acid hydrolysis and shaking were determined as a response; moreover, we evaluated the ability to concentrate. The test formulations are shown in Table 7.

TABLE 7
Test formulations.

| | | | | | Theoretical |
|---|---|---|---|---|---|
| | | | | | Osm of |
| | Desig- | | | | osmotic |
| No. | nation | Formulation | | | agent, mOsm/l |
| 1 | Hist | anti-TRBV9 | 1.5-90* | mg/ml | 13 |
| | | L-histidine | 0.517 | mg/ml | |
| | | L-histidine h/c | | | |
| | | monohydrate | 0.35 | mg/ml | |
| | | Water for injection | to 1 | ml | |
| 2 | Tre | anti-TRBV9 | 1.5-50* | mg/ml | 264 |
| | | L-histidine | 0.517 | mg/ml | |
| | | L-histidine h/c | 350 | mg/ml | |
| | | monohydrate | | | |
| | | Trehalose dihydrate | 100 | mg/ml | |
| | | Water for injection | to 1 | ml | |
| 3 | Sorb | anti-TRBV9 | 1.5-90* | mg/ml | 274 |
| | | L-histidine | 0.517 | mg/ml | |
| | | L-histidine h/c | 0.35 | mg/ml | |
| | | monohydrate | | | |
| | | Sorbitol | 50 | mg/ml | |
| | | Water for injection | to 1 | ml | |
| 4 | Prol | anti-TRBV9 | 1.5-90* | mg/ml | 253 |
| | | L-histidine | 0.517 | mg/ml | |
| | | L-histidine h/c | 0.35 | mg/ml | |
| | | Monohydrate | | | |
| | | L-proline | 27 | mg/ml | |
| | | Water for injection | to 1 | ml | |
| 5 | NaCl 9.0 | anti-TRBV9 | 1.5-90* | mg/ml | 308 |
| | | L-histidine | 0.517 | mg/ml | |
| | | L-histidine h/c | 0.35 | mg/ml | |
| | | Monohydrate | | | |
| | | Sodium chloride | 9.0 | mg/ml | |
| | | Water for injection | to 1 | ml | |
| 6 | NaCl 4.5 | anti-TRBV9 | 1.5-90* | mg/ml | 154 |
| | | L-histidine | 0.517 | mg/ml | |
| | | L-histidine h/c | 0.35 | mg/ml | |
| | | monohydrate | | | |
| | | Sodium chloride | 4.5 | mg/ml | |
| | | Water for injection | to 1 | ml | |

*max protein concentration is limited by direct concentrating.

### Study of Stability Prediction Indicators.

Results of the determination of aggregation temperature by method 3, melting point by method 4, diffusion interaction parameter by method 6 and ability to concentrate by method 1 are shown in Table 8.

TABLE 8
Results of study of stability and direct concentrating of anti-TRBV9 in various formulations.

| | Test formulation, mg/ml | | | | | Direct concentrating | | | | | |
|---|---|---|---|---|---|---|---|---|---|---|---|
| | | Formulation | Tm, | Tag, | | C in, | V in, | C fin., | V fin., | | Cfin./ |
| No. | Protein | designation | ° C. | ° C. | kd | mg/ml | ml | mg/ml | ml | Yield, % | Cin. |
| 1 | 5* | Hist | 66.3 | 64.33 | 3.07E−02 | 37.2 | 600 | 90.7 | 187 | 76 | 2.4 |
| 2 | 5* | Tre | 67.8 | 67.01 | 5.12E−02 | 16.9 | 600 | 57.6 | 172 | 97 | 3.4 |
| 3 | 5* | Sorb | 66.9 | 64.87 | 3.87E−02 | 32.4 | 600 | 91.8 | 170 | 80 | 2.8 |
| 4 | 5* | Prol | 66.4 | 64.5 | 4.09E−02 | 39.4 | 600 | 91.4 | 170 | 66 | 2.3 |
| 5 | 5* | NaCl 9.0 | 64.4 | 58.8 | −3.86E−03 | 24.1 | 600 | 90.7 | 129 | 81 | 3.8 |
| 6 | 5* | NaCl 4.5 | 64.6 | 60.69 | −4.73E−03 | 28.4 | 600 | 83.0 | 145 | 71 | 2.9 |

*Protein concentration is method-specific and given in the method description.

### Determination of Thermal Stability.

Thermal stability was assessed by method 7. Before and following thermal stress, we determined: purity by SE HPLC by method 13, purities by electrophoresis in non-reducing conditions by method 17, charge variant profile in capillary by method 14, hydrodynamic radius by method 5; absolute change in charge variant profile was calculated by method 22. The results are shown in Table 9.

TABLE 9
Results of study of stability of anti-TRBV9 in various formulations under thermal stress.

| | | | | | | | | | Labchip | | | | | | | | |
|---|---|---|---|---|---|---|---|---|---|---|---|---|---|---|---|---|---|
| | | | | | SE HPLC | | | | non-red. | | | | | | | | |
| | | | Hydrodynamic | | Aggregates, | | Monomer, | | Monomer, | | Labchip charge variant profile, % | | | | | | |
| | Test formulation, | | radius, nm | | % | | % | | % | | | | | | | | Δ |
| | mg/ml | | | Δ | | Δ | | Δ | | Δ | | | | | | | abs. |
| | | Formulation | | TS50 | | TS50 | | TS50 | | TS50 | Alc. | Base | Acid. | Alc. | Base | Acid. | TS50 |
| No. | Protein | designation | IC | 96H | IC | 96H | IC | 96H | IC | 96H | IC | | | Δ TS50 96H | | | 96H |
| 1 | 5* | Hist | 3.3 | 0.9 | 0.92 | 0.31 | 98.12 | 1 | 93.1 | 1.9 | 7.63 | 61.62 | 30.75 | 9.83 | −12.17 | 2.35 | 24.35 |
| 2 | 5* | Tre | 5.6 | −0.8 | 0.79 | 0.35 | 98.34 | −1.07 | 93.1 | 0.3 | 7.2 | 62.97 | 29.83 | 9.81 | −14.64 | 4.83 | 29.28 |
| 3 | 5* | Sorb | 3.6 | 0.9 | 0.83 | 0.29 | 98.17 | −1.1 | 91 | 2.1 | 8.12 | 63.99 | 27.89 | 9.07 | −15.53 | 6.46 | 31.06 |
| 4 | 5* | Prol | 3.5 | 3.6 | 0.76 | 0.28 | 98.23 | 1.02 | 91.1 | 2 | 7.84 | 64.56 | 27.60 | 9.63 | −9.47 | −0.16 | 19.26 |
| 5 | 5* | NaCl 9.0 | 5.7 | 1.6 | 0.84 | 1.71 | 98.08 | −2.37 | 93.7 | −0.7 | 9.42 | 65.88 | 24.70 | 9.32 | −10.67 | 1.35 | 21.34 |
| 6 | 5* | NaCl 4.5 | 5.7 | 1.6 | 0.86 | 1.46 | 98.05 | −2.17 | 93.8 | −0.7 | 8.66 | 65.54 | 25.81 | 6.65 | −8.89 | 2.23 | 17.77 |

*Protein concentration is method-specific and given in the method description.

### Determination of Stability Under Acid Hydrolysis.

Stability under acid hydrolysis was assessed by method 10. Before and following hydrolysis, we determined: purity by SE HPLC by method 13, purity by electrophoresis in non-reducing conditions by method 17, charge variant profile in capillary by method 14, hydrodynamic radius by method 5; absolute change in charge variant profile was calculated by method 22. The results are shown in Table 10.

TABLE 10
Results of study of stability of anti-TRBV9 in various formulations under acid hydrolysis.

| | | | | | | | | | Labchip | | | | | | | | |
|---|---|---|---|---|---|---|---|---|---|---|---|---|---|---|---|---|---|
| | Test | | | | SE HPLC | | | | non-red. | | | | | | | | |
| | formulation, | | Hydrodynamic | | Aggregates, | | Monomer, | | Monomer, | | Labchip charge variant profile, % | | | | | | |
| | mg/ml | | radius, nm | | % | | % | | % | | | | | | | | Δ |
| | | For- | | Δ | | Δ | | Δ | | Δ | | | | | | | abs. |
| | | mulation | | Acid | | Acid | | Acid | | Acid | | | | | | | Acid |
| | | desig- | | 3.0 | | 3.0 | | 3.0 | | 3.0 | Alc. | Base | Acid. | Alc. | Base | Acid. | 3.0 |
| No. | Protein | nation | IC | 24H | IC | 24H | IC | 24H | IC | 24H | IC | | | Δ Acid 3.0 24H | | | 24H |
| 1 | 5* | 5 mM | 3.3 | 32.2 | 0.92 | 0.1 | 98.12 | −0.19 | 93.1 | 1.0 | 7.63 | 61.62 | 30.75 | −1.13 | 2.92 | −1.78 | 5.83 |
| | | His, | | | | | | | | | | | | | | | |
| | | pH = 6.3 | | | | | | | | | | | | | | | |
| 2 | 5* | Tre | 5.6 | 12.8 | 0.79 | 1.65 | 98.34 | −1.97 | 93.1 | −0.1 | 7.2 | 62.97 | 29.83 | 0.22 | 0.64 | −0.86 | 1.72 |
| 3 | 5* | Sorb | 3.6 | 16.9 | 0.83 | 3.36 | 98.17 | −3.81 | 91 | 2.4 | 8.12 | 63.99 | 27.89 | 0.53 | −0.4 | −0.13 | 1.06 |
| 4 | 5* | Prol | 3.5 | 25.2 | 0.76 | 1.21 | 98.23 | −1.4 | 91.1 | 2.1 | 7.84 | 64.56 | 27.60 | 1.57 | −0.4 | −1.16 | 3.13 |
| 5 | 5* | NaCl | 5.7 | 238.8 | 0.84 | n/a | 98.08 | n/a | 93.7 | n/a | 9.42 | 65.88 | 24.70 | n/a | | | n/a |
| | | 9.0 | | | | | | | | | | | | | | | |
| 6 | 5* | NaCl | 5.7 | 4.9 | 0.86 | 24.96 | 98.05 | −25.56 | 93.8 | −0.5 | 8.66 | 65.54 | 25.81 | 18.17 | −14.88 | −3.31 | 36.36 |
| | | 4.5 | | | | | | | | | | | | | | | |

*Protein concentration is method-specific and given in the method description.

### Determination of Stability Under Shaking.

Stability under shaking was assessed by method 8. Before and following shaking, we determined: purity by SE HPLC by method 13, purity by electrophoresis in non-reducing conditions by method 17, charge variant profile in capillary by method 14, hydrodynamic radius by method 5; absolute change in charge variant profile was calculated by method 22. The results are shown in Table 11.

TABLE 11
Results of study of stability of anti-TRBV9 in various formulations under shaking.

| | | | | | | | | | Labchip | | | | | | | | |
|---|---|---|---|---|---|---|---|---|---|---|---|---|---|---|---|---|---|
| | Test | | DLS | | SE HPLC | | | | non-red. | | | | | | | | |
| | formulation, | | Z-average, | | Aggregates, | | Monomer, | | Monomer, | | Labchip charge variant profile, % | | | | | | |
| | mg/ml | | nm | | % | | % | | % | | | | | | | | Δ |
| | | Formulation | | Δ | | Δ | | Δ | | Δ | | | | | | | abs. |
| | | desig- | | SH800 | | SH800 | | SH800 | | SH800 | Alc. | Base | Acid. | Alc. | Base | Acid. | SH800 |
| No. | Protein | nation | IC | 96H | IC | 96H | IC | 96H | IC | 96H | IC | | | Δ SH800 96H | | | 96H |
| 1 | 5* | Hist | 3.3 | 0.4 | 0.92 | −0.03 | 98.12 | 0.1 | 93.1 | −0.3 | 7.63 | 61.62 | 30.75 | 0.01 | −0.16 | 0.14 | 0.31 |
| 2 | 5* | Tre | 5.6 | 12.7 | 0.79 | −0.08 | 98.34 | 0.07 | 93.1 | −2.7 | 7.2 | 62.97 | 29.83 | −0.40 | 0.79 | −0.38 | 1.57 |
| 3 | 5* | Sorb | 3.6 | 1.3 | 0.83 | −0.03 | 98.17 | 0.02 | 91 | 1.3 | 8.12 | 63.99 | 27.89 | −0.35 | −0.08 | 0.43 | 0.86 |
| 4 | 5* | Prol | 3.5 | 0.5 | 0.76 | −0.02 | 98.23 | 0.05 | 91.1 | 0 | 7.84 | 64.56 | 27.60 | −0.21 | 1.51 | −1.30 | 3.02 |
| 5 | 5* | NaCl 9.0 | 5.7 | −0.3 | 0.84 | 0.02 | 98.08 | 0.02 | 93.7 | −2 | 9.42 | 65.88 | 24.70 | −0.43 | −0.56 | 1.00 | 1.99 |
| 6 | 5* | NaCl 4.5 | 5.7 | −0.2 | 0.86 | −0.01 | 98.05 | 0.07 | 93.8 | 0.7 | 8.66 | 65.5 | 25.81 | −0.70 | 1.41 | −0.72 | 2.83 |

*Protein concentration is method-specific and given in the method description.

The given formulations demonstrated a high melting point, high aggregation temperature, as well as an acceptable k_{D} value. The given formulations showed high concentration values when concentrated directly and, according to the results of control, there is a slight change in the quality of protein under thermal stress and shaking (control by SE HPLC). Furthermore, the given formulations demonstrate an insignificant value of the absolute change in the acid-base profile under shaking and acid hydrolysis.

### Example 4. Selection of Osmotic Agent with Added Stabilizers

The present study employs histidine buffer system as the basis of the pharmaceutical composition. Excipients suitable for parenteral administration, which are listed in Table 12, were studied to be used as stabilizers.

To ensure physiological osmolality of compositions, the content of osmotic agents was reduced taking into account the contribution of stabilizers into osmolality of solutions.

As part of the study, we assessed the effect of the resulting formulations on colloidal and conformational stability of the protein. Aggregation temperature, melting point, diffusion interaction parameter, changes in purity and in acid-base profile following thermal stress, shaking, acid hydrolysis, several freeze-thaw cycles were determined as response.

TABLE 12
Test formulations in 5 mM histidine buffer solution with pH 6.3.

| | | Formulation | | | | | | | | |
|---|---|---|---|---|---|---|---|---|---|---|
| | | | Osmolytic agent | | | Stabilizer | | | General | |
| | | | | | Theor. | | | Theor. | theor. | |
| | | Protein, | | C, | Osm, | | C, | Osm, | Osm, | |
| No. | Designation | mg/ml | Type | mg/ml | mOsm/kg | Type | mg/ml | mosm/kg | mOsm/kg | Stabilizer effect |
| 1 | Sorb Arg 100 | 1.5-* | D-sorbitol | 27 | 148 | L-arginine | 21.07 | 174 | 322 | Increases protein thermal |
| | mM | | | | | hydrochloride | | | | stability, solubility. |
| | | | | | | | | | | Contributes to reduction of |
| | | | | | | | | | | viscosity. Exhibits osmotic |
| | | | | | | | | | | properties. |
| 2 | Sorb Meth 10 | 1.5-* | D-sorbitol | 50 | 274 | L- | 1.49 | 10 | 284 | Antioxidant effect. |
| | mM | | | | | methionine | | | | |
| 2′ | Sorb Meth 10 | 1.5-* | D-sorbitol | 50 | 274 | L- | 1.49 | 10 | 284 | Antioxidant effect. |
| | mM | | | | | methionine | | | | |
| 3 | Sorb Meth 30 | 1.5-* | D-sorbitol | 50 | 274 | L- | 4.48 | 30 | 304 | Antioxidant effect. |
| | mM | | | | | methionine | | | | |
| 3′ | Sorb Meth 30 | 1.5-* | D-sorbitol | 50 | 274 | L- | 4.48 | 30 | 304 | Antioxidant effect. |
| | mM | | | | | methionine | | | | |
| 4 | Sorb Gly 100 | 1.5-* | D-sorbitol | 36.1 | 198 | L-glycine | 7.51 | 98 | 296 | Increases protein thermal |
| | mM | | | | | | | | | stability, solubility. |
| | | | | | | | | | | Exhibits osmotic properties. |
| 4′ | Sorb Gly 100 | 1.5-* | D-sorbitol | 36.1 | 198 | L-glycine | 7.51 | 98 | 296 | Increases protein thermal |
| | mM | | | | | | | | | stability, solubility. |
| | | | | | | | | | | Exhibits osmotic properties. |
| 5 | Sorb PS80 | 1.5-* | D-sorbitol | 50 | 274 | Poloxamer | 0.50 | — | 274 | Nonionic surfactant. Prevents |
| | | | | | | 188 | | | | protein aggregation under |
| | | | | | | | | | | freeze-drying, freeze-thaw |
| | | | | | | | | | | cycle. Suppresses formation |
| | | | | | | | | | | of aggregates at phase |
| | | | | | | | | | | boundaries under stirring and |
| | | | | | | | | | | shaking. |
| 6 | Prol Gly 100 | 1.5-* | L-proline | 21 | 182 | L-glycine | 7.51 | 98 | 280 | Increases protein thermal |
| | mM | | | | | | | | | stability, solubility. |
| | | | | | | | | | | Exhibits osmotic properties. |
| 6′ | Prol Gly 100 | 1.5-* | L-proline | 21 | 182 | L-glycine | 7.51 | 98 | 280 | Increases protein thermal |
| | mM | | | | | | | | | stability, solubility. |
| | | | | | | | | | | Exhibits osmotic properties. |
| 7 | Prol Meth 10 | 1.5-* | L-proline | 27 | 235 | L- | 1.49 | 10 | 245 | Antioxidant effect. |
| | mM | | | | | methionine | | | | |
| 8 | Prol 30 | 1.5-* | L-proline | 30 | 260 | — | — | — | 260 | — |
| 9 | Prol 27 | 1.5-* | L-proline | 27 | 235 | — | — | — | 235 | — |
| 10 | Prol | 1.5-* | L-proline | 30 | 260 | L- | 1.49 | 10 | 270 | Antioxidant effect. |
| | Meth 10 mM | | | | | methionine | | | | |
| 11 | Prol | 1.5-* | L-proline | 13 | 113 | L-arginine | 21.07 | 174 | 287 | Increases protein thermal |
| | Arg 100 mM | | | | | hydrochloride | | | | stability, solubility. |
| | | | | | | | | | | Contributes to reduction of |
| | | | | | | | | | | viscosity. Exhibits osmotic |
| | | | | | | | | | | properties. |
| 12 | Prol | 1.5-* | L-proline | 30 | 260 | Polysorbate | 0.55 | — | 260 | Nonionic surfactant. Prevents |
| | PS80 | | | | | 80 | | | | protein aggregation under |
| 13 | Prol | 1.5-* | L-proline | 30 | 260 | Poloxamer | 0.55 | — | 260 | freeze-drying, freeze-thaw |
| | Koll | | | | | 188 | | | | cycle. Suppresses formation |
| | | | | | | | | | | of aggregates at phase |
| | | | | | | | | | | boundaries under stirring and |
| | | | | | | | | | | shaking. |
| 14 | Prol | 1.5-* | L-proline | 30 | 260 | Polypropylene | 0.55 | — | 260 | Diatomic alcohol, polymer of |
| | PPG | | | | | glycol | | | | propylene glycol. Used as |
| | | | | | | | | | | hygroscopic agent, carrier |
| | | | | | | | | | | element and solvent. |

* max protein concentration is limited by direct concentrating.
Formulations 2′, 3′, 4′ and 6′ are repeats of formulations 2, 3, 4 and 6, respectively.

### Study of Stability Prediction Indicators.

Melting point was determined by method 4, aggregation point was determined by method 3. Diffusion interaction parameter was determined by method 6. Samples were concentrated according to method 1. Viscosity was measured according to method 19. The results are shown in Table 13.

TABLE 13
Results of study of stability and direct concentrating of anti-TRBV9, in various formulations.

| | | | | | | Direct concentrating | | | | | | | |
|---|---|---|---|---|---|---|---|---|---|---|---|---|---|
| | Test formulation, mg/ml | | | | | | | C | V | | | | |
| | | Formulation | Tm, | Tag, | | C in, | V in, | fin., | fin., | Yield, | Cfin./ | C η, | |
| No. | Protein | designation | ° C. | ° C. | kd | mg/ml | μl | mg/ml | ml | % | Cin. | mg/ml* | η |
| 1 | 5** | Sorb Arg 100 mM | 64.9 | 61.04 | −3.44E−03 | 21 | 2100 | 58 | 150 | 70 | 2.8 | n/a | n/a |
| 2 | 5** | Sorb Meth 10 mM | 67.1 | 64.82 | 5.36E−02 | 31 | 1430 | 61 | 168 | 56 | 2 | n/a | n/a |
| 2′ | 5** | Sorb Meth 10 mM | n/a | 66 | n/a | 29 | 1732 | 100 | 400 | 80 | 3.4 | 100 | 2.33 |
| 3 | 5** | Sorb Meth 30 mM | 67 | 64.44 | 6.42E−02 | 33 | 1400 | 68 | 195 | 68 | 2.1 | n/a | n/a |
| 3′ | 5** | Sorb Meth 30 mM | n/a | 66.3 | n/a | 29 | 1719 | 103 | 400 | 83 | 3.6 | 103 | 6.20 |
| 4 | 5** | Sorb Gly 100 mM | 67.1 | 65.8 | 3.09E−02 | 27 | 1500 | 86 | 170 | 90 | 3.2 | n/a | n/a |
| 4′ | 5** | Sorb Gly 100 mM | n/a | 66.54 | n/a | 26 | 1959 | 96 | 400 | 75 | 3.7 | 96 | 5.22 |
| 5 | 5** | Sorb PS80 | 66.8 | 65.91 | 2.65E−02 | 41 | 1200 | 80 | 213 | 70 | 2 | n/a | n/a |
| 6 | 5** | Prol Gly 100 mM | n/a | 60.55 | n/a | 34 | 1478 | 119 | 400 | 95 | 3.5 | 119 | 6.31 |
| 6′ | 5** | Prol Gly 100 mM | n/a | 65.8 | 8.97E−02 | 19 | 1200 | 73 | 275 | 111 | 3.8 | 73 | 2.33 |
| 7 | 5** | Prol Meth 10 mM | n/a | 57.41 | n/a | 33 | 1539 | 94 | 350 | 65 | 2.8 | 94 | 5.56 |
| 8 | 5** | Prol 30 | n/a | 65.8 | 1.38E−01 | 21 | 2200 | 76 | 550 | 108 | 3.6 | 76 | 2.41 |
| 9 | 5** | Prol 27 | n/a | 65.4 | 1.17E−01 | 29 | 1200 | 85 | 368 | 111 | 2.9 | 85 | 2.89 |
| 10 | 5** | Prol Meth 10 mM | n/a | 65.3 | 9.47E−02 | 34 | 1200 | 97 | 395 | 108 | 2.9 | 97 | 3.64 |
| 11 | 5** | Prol Arg 100 mM | n/a | 61.9 | −4.65E−03 | 29 | 1200 | 73 | 430 | 109 | 2.5 | 73 | 1.90 |

Formulations 2′, 3′, 4′ and 6′ are repeats of formulations 2, 3, 4 and 6, respectively.
*Concentration of a sample for which viscosity was determined.
**Protein concentration is method-specific and given in the method description.

### Determination of Thermal Stability.

Thermal stability was assessed by method 7. Before and following thermal stress, we determined: purity by SE HPLC by method 13, charge variant profile in capillary by method 14, hydrodynamic radius by method 5; absolute change in charge variant profile was calculated by method 22. The results are shown in Table 14.

TABLE 14
Results of study of stability of anti-TRBV9 in various formulations under thermal stress.

| | Test formulation, | | Hydrodynamic | | SE HPLC | | | | Labchip charge variant profile, % | | | | | | |
|---|---|---|---|---|---|---|---|---|---|---|---|---|---|---|---|
| | mg/ml | | radius, nm | | Aggregates, % | | Monomer, % | | | | | | | | Δ |
| | | For- | | Δ | | Δ | | Δ | | | | | | | abs. |
| | | mulation | | TS50 | | TS50 | | TS50 | Alc. | Base | Acid. | Alc. | Base | Acid. | TS50 |
| No. | Protein | designation | IC | 96H | IC | 96H | IC | 96H | IC | | | Δ TS50 96H | | | 96H |
| 1 | 5* | Sorb Arg | 6.1 | −1.9 | 0.73 | 1.23 | 98.2 | −1.91 | 9.49 | 62.70 | 27.81 | 5.54 | −8.72 | 3.18 | 17.44 |
| | | 100 mM | | | | | | | | | | | | | |
| 2 | 5* | Sorb Meth | 4.1 | 0.1 | 0.76 | 0.21 | 98.18 | −0.94 | 9.64 | 61.54 | 28.82 | 6.29 | −7.53 | 1.24 | 15.06 |
| | | 10 mM | | | | | | | | | | | | | |
| 2′ | 5* | Sorb Meth | 4.3 | 0 | 0.85 | 0.22 | 97.2 | −0.18 | 11.11 | 65.67 | 23.22 | 12.10 | −16.11 | 4.01 | 32.22 |
| | | 10 mM | | | | | | | | | | | | | |
| 3 | 5* | Sorb Meth | 4.3 | 0.7 | 0.74 | 0.24 | 98.17 | −1 | 7.47 | 63.27 | 29.25 | 12.46 | −15.12 | 2.67 | 30.25 |
| | | 30 mM | | | | | | | | | | | | | |
| 3′ | 5* | Sorb Meth | 4.1 | 0.3 | 0.89 | 0.27 | 97.7 | −0.99 | 10.86 | 65.22 | 23.92 | 12.21 | −16.35 | 4.14 | 32.7 |
| | | 30 mM | | | | | | | | | | | | | |
| 4 | 5* | Sorb Gly | 3.7 | 0.3 | 0.83 | 0.16 | 97.95 | −1.02 | 7.22 | 64.35 | 28.43 | 13.08 | −13.42 | 0.34 | 26.84 |
| | | 100 m | | | | | | | | | | | | | |
| 4′ | 5* | Sorb Gly | 4.4 | 0.1 | 0.82 | 0.18 | 97.8 | −0.98 | 11.06 | 65.71 | 23.23 | 11.80 | −15.60 | 3.80 | 31.2 |
| | | 100 mM | | | | | | | | | | | | | |
| 5 | 5* | Sorb PS80 | 3.8 | 0.4 | 0.95 | 0.27 | 97.85 | −1.08 | 9.02 | 63.40 | 25.57 | 10.95 | −16.06 | 7.12 | 34.13 |
| 6 | 5* | Prol Gly | 3.9 | 0.8 | 0.89 | 0.32 | 97.7 | −1.21 | 11.14 | 64.45 | 24.41 | 11.01 | −12.95 | 1.94 | 25.9 |
| | | 100 mM | | | | | | | | | | | | | |
| 6′ | 5* | Prol Gly | 4.3 | 0.1 | 0.67 | −0.01 | 98.04 | −0.8 | 13.56 | 63.22 | 23.23 | 10.61 | −11.65 | 1.05 | 23.31 |
| | | 100 mM | | | | | | | | | | | | | |
| 7 | 5* | Prol Meth | 4 | 0 | 0.89 | 0.23 | 97.8 | −0.94 | 11.21 | 65.57 | 23.22 | 11.79 | −13.41 | 1.62 | 26.82 |
| | | 10 mM | | | | | | | | | | | | | |
| 8 | 5* | Prol 30 | 4.6 | 0.3 | 0.71 | 0.13 | 98.01 | −0.69 | 11.92 | 64.99 | 23.10 | 12.57 | −13.68 | 1.10 | 27.35 |
| 9 | 5* | Prol 27 | 4.6 | 0 | 0.68 | 0.11 | 98.09 | −0.67 | 14.90 | 63.84 | 21.26 | 10.85 | −11.73 | 0.88 | 23.46 |
| 10 | 5* | Prol Meth | 4.4 | 0.4 | 0.78 | −0.16 | 98.18 | −0.89 | 14.00 | 63.23 | 22.77 | 10.43 | −11.48 | 1.05 | 22.96 |
| | | 10 mM | | | | | | | | | | | | | |
| 11 | 5* | Prol Arg | 5.8 | 1 | 0.67 | 1.14 | 98.11 | −1.74 | 14.06 | 63.70 | 22.25 | 9.12 | −9.06 | −0.07 | 18.25 |
| | | 100 mM | | | | | | | | | | | | | |
| 12 | 5* | Prol PS80 | n/a | n/a | 0.66 | 0.34 | 97.97 | −0.93 | 13.45 | 62.23 | 24.33 | 11.20 | −11.88 | 0.68 | 23.76 |
| 13 | 5* | Prol Koll | n/a | n/a | 0.65 | 0.35 | 98.1 | −1.03 | 13.67 | 62.81 | 23.53 | 10.82 | −11.52 | 0.69 | 23.03 |
| 14 | 5* | Prol PPG | n/a | n/a | 0.58 | 0.14 | 98.03 | −0.78 | 14.00 | 62.82 | 23.19 | 10.99 | −11.88 | 0.88 | 23.75 |

*Protein concentration is method-specific and given in the method description.
Formulations 2′, 3′, 4′ and 6′ are repeats of formulations 2, 3, 4 and 6, respectively.

### Determination of Stability Under Acid Hydrolysis.

M Stability under acid hydrolysis was assessed by method 10. Before and following hydrolysis, we determined: purity by SE HPLC by method 13, charge variant profile in capillary by method 14, hydrodynamic radius by method 5; absolute change in charge variant profile was calculated by method 22. The results are shown in Table 15.

TABLE 15
Results of study of stability of anti-TRBV9 in various formulations under acid hydrolysis.

| | Test | | Hydrodynamic | | SE HPLC | | | | | | | | | | |
|---|---|---|---|---|---|---|---|---|---|---|---|---|---|---|---|
| | formulation, | | radius, nm | | Aggregates, % | | Monomer, % | | Labchip charge variant profile, % | | | | | | |
| | mg/ml | | | Δ | | Δ | | Δ | | | | | | | Δ abs. |
| | | For- | | Acid | | Acid | | Acid | | | | | | | Acid |
| | Pro- | mulation | | 3.0 | | 3.0 | | 3.0 | Alc. | Base | Acid. | Alc. | Base | Acid. | 3.0 |
| No. | tein | designation | IC | 96H | IC | 24H | IC | 24H | IC | | | Δ Acid 3.0 24H | | | 24H |
| 1 | 5* | Sorb Arg | 6.1 | 8 | 0.73 | 1.974 | 98.2 | −2.098 | 9.49 | 62.70 | 27.81 | 0.28 | −0.85 | 0.57 | 1.7 |
| | | 100 mM | | | | | | | | | | | | | |
| 2 | 5* | Sorb Meth | 4.1 | 9.2 | 0.76 | 0.007 | 98.18 | −0.115 | 9.64 | 61.54 | 28.82 | −1.07 | 0.34 | 0.73 | 2.14 |
| | | 10 mM | | | | | | | | | | | | | |
| 2′ | 5* | Sorb Meth | 4.3 | n/a | 0.85 | n/a | 97.2 | n/a | 11.11 | 65.67 | 23.22 | n/a | n/a | n/a | n/a |
| | | 10 mM | | | | | | | | | | | | | |
| 3 | 5* | Sorb Meth | 4.3 | 6.6 | 0.74 | 0.105 | 98.17 | −0.195 | 7.47 | 63.27 | 29.25 | 1.00 | 0.91 | −1.90 | 3.81 |
| | | 30 mM | | | | | | | | | | | | | |
| 3′ | 5* | Sorb Meth | 4.1 | n/a | 0.89 | n/a | 97.7 | n/a | 10.86 | 65.22 | 23.92 | n/a | n/a | n/a | n/a |
| | | 30 mM | | | | | | | | | | | | | |
| 4 | 5* | Sorb Gly | 3.7 | 9.3 | 0.83 | 0.049 | 97.95 | −0.091 | 7.22 | 64.35 | 28.43 | 2.39 | 0.48 | −2.87 | 5.74 |
| | | 100 mM | | | | | | | | | | | | | |
| 4′ | 5* | Sorb Gly | 4.4 | n/a | 0.82 | n/a | 97.8 | n/a | 11.06 | 65.71 | 23.23 | n/a | n/a | n/a | n/a |
| | | 100 mM | | | | | | | | | | | | | |
| 5 | 5* | Sorb PS80 | 3.8 | 5.7 | 0.95 | 4.135 | 97.85 | −4.551 | 9.02 | 63.40 | 25.57 | 0.79 | −5.87 | 7.09 | 13.75 |
| 6 | 5* | Prol Gly | 3.9 | n/a | 0.89 | n/a | 97.7 | n/a | n/a | n/a | n/a | n/a | n/a | n/a | n/a |
| | | 100 mM | | | | | | | | | | | | | |
| 6′ | 5* | Prol Gly | 4.3 | n/a | 0.67 | 3.67 | 98.04 | −3.88 | 13.56 | 63.22 | 23.23 | 3.33 | −2.74 | −0.59 | 2.48 |
| | | 100 mM | | | | | | | | | | | | | |
| 7 | 5* | Prol Meth | 4 | n/a | 0.89 | n/a | 97.8 | n/a | n/a | n/a | n/a | n/a | n/a | n/a | n/a |
| | | 10 mM | | | | | | | | | | | | | |
| 8 | 5* | Prol 30 | 4.6 | 43 | 0.71 | 3.35 | 98.01 | −3.55 | 11.92 | 64.99 | 23.10 | −0.36 | −1.33 | 1.69 | 4.84 |
| 9 | 5* | Prol 27 | 4.6 | 5.8 | 0.68 | 4.64 | 98.09 | −4.87 | 14.90 | 63.84 | 21.26 | 0.89 | −0.06 | −0.84 | 2.99 |
| 10 | 5* | Prol Meth | 4.4 | 7.2 | 0.78 | 12.77 | 98.18 | −13.4 | 14.00 | 63.23 | 22.77 | −14.06 | −63.70 | −22.25 | 4.08 |
| | | 10 mM | | | | | | | | | | | | | |
| 11 | 5* | Prol Arg | 5.8 | 55.6 | 0.67 | n/a | 98.11 | n/a | 14.06 | 63.70 | 22.25 | 0.51 | −1.69 | 1.17 | 16.38 |
| | | 100 mM | | | | | | | | | | | | | |
| 12 | 5* | Prol PS80 | n/a | n/a | 0.66 | 3.53 | 97.97 | −3.65 | 13.45 | 62.23 | 24.33 | 2.00 | −2.93 | 0.91 | 1.2 |
| 13 | 5* | Prol Koll | n/a | n/a | 0.65 | 5.85 | 98.1 | −6.1 | 13.67 | 62.81 | 23.53 | 0.62 | −1.76 | 1.14 | 3.88 |
| 14 | 5* | Prol PPG | n/a | n/a | 0.58 | 4.95 | 98.03 | −5.26 | 14.00 | 62.82 | 23.19 | −0.36 | −1.33 | 1.69 | 3.09 |

*Protein concentration is method-specific and given in the method description.
Formulations 2′, 3′, 4′ and 6′ are repeats of formulations 2, 3, 4 and 6, respectively.

### Determination of Stability Under Shaking

Stability under shaking was assessed by method 8. Before and following shaking, we determined: purity by SE HPLC by method 13, charge variant profile in capillary by method 14, hydrodynamic radius by method 5; absolute change in charge variant profile was calculated by method 22. The results are shown in Table 16.

TABLE 16
Results of study of stability of anti-TRBV9 in various formulations under shaking.

| | Test formulation, | | Hydrodynamic | | SE HPLC | | | | Labchip charge variant profile, % | | | | | | |
|---|---|---|---|---|---|---|---|---|---|---|---|---|---|---|---|
| | mg/ml | | radius, nm | | Aggregates, % | | Monomer, % | | | | | | | | Δ |
| | | For- | | Δ | | Δ | | Δ | | | | | | | abs. |
| | Pro- | mulation | | SH800 | | SH800 | | SH800 | Alc. | Base | Acid. | Alc. | Base | Acid. | SH800 |
| No. | tein | designation | IC | 96H | IC | 96H | IC | 96H | IC | | | Δ SH800 96H | | | 96H |
| 1 | 5* | Sorb Arg 100 | 6.1 | −0.2 | 0.73 | −0.04 | 98.2 | 0.09 | 9.49 | 62.70 | 27.81 | −0.26 | 0.42 | −0.15 | 0.83 |
| 2 | 5* | Sorb Meth 10 | 4.1 | 0.9 | 0.76 | −0.02 | 98.18 | 0 | 9.64 | 61.54 | 28.82 | −0.68 | 1.18 | −0.5 | 2.36 |
| | | mM | | | | | | | | | | | | | |
| 2′ | 5* | Sorb Meth 10 | 4.3 | n/a | 0.85 | n/a | 97.2 | n/a | 11.11 | 65.67 | 23.22 | n/a | n/a | n/a | n/a |
| | | mM | | | | | | | | | | | | | |
| 3 | 5* | Sorb Meth 30 | 4.3 | 0.6 | 0.74 | −0.02 | 98.17 | 0.05 | 7.47 | 63.27 | 29.25 | −0.13 | −0.43 | 0.57 | 1.13 |
| | | mM | | | | | | | | | | | | | |
| 3′ | 5* | Sorb Meth 30 | 4.1 | n/a | 0.89 | n/a | 97.7 | n/a | 10.86 | 65.22 | 23.92 | n/a | n/a | n/a | n/a |
| | | mM | | | | | | | | | | | | | |
| 4 | 5* | Sorb Gly 100 | 3.7 | 0.6 | 0.83 | −0.1 | 97.95 | 0.19 | 7.22 | 64.35 | 28.43 | 0.39 | −0.91 | 0.52 | 1.82 |
| | | m | | | | | | | | | | | | | |
| 4′ | 5* | Sorb Gly 100 | 4.4 | n/a | 0.82 | n/a | 97.8 | n/a | 11.06 | 65.71 | 23.23 | n/a | n/a | n/a | n/a |
| | | mM | | | | | | | | | | | | | |
| 5 | 5* | Sorb PS80 | 3.8 | 0.3 | 0.95 | −0.01 | 97.85 | 0.05 | 9.02 | 63.40 | 25.57 | −0.46 | −0.18 | 2.65 | 3.29 |
| 6 | 5* | Prol Gly 100 | 3.9 | n/a | 0.89 | n/a | 97.7 | n/a | 11.14 | 64.45 | 24.41 | n/a | n/a | n/a | n/a |
| | | mM | | | | | | | | | | | | | |
| 6′ | 5* | Prol Gly 100 | 4.3 | 2 | 0.67 | −0.04 | 98.04 | 0.08 | 13.56 | 63.22 | 23.23 | 0.44 | 0.69 | −1.14 | 2.27 |
| | | mM | | | | | | | | | | | | | |
| 7 | 5* | Prol Meth 10 | 4 | n/a | 0.89 | n/a | 97.8 | n/a | 11.21 | 65.57 | 23.22 | n/a | n/ | n/a | n/a |
| | | mM | | | | | | | | | | | | | |
| 8 | 5* | Prol 30 | 4.6 | −0.6 | 0.71 | −0.03 | 98.01 | 0.05 | 11.92 | 64.99 | 23.10 | 1.56 | −2.04 | 0.46 | 4.06 |
| 9 | 5* | Prol 27 | 4.6 | 0 | 0.68 | −0.04 | 98.09 | 0.05 | 14.90 | 63.84 | 21.26 | −1.04 | −0.83 | 1.89 | 3.76 |
| 10 | 5* | Prol Meth 10 | 4.4 | 0.1 | 0.78 | −0.07 | 98.18 | −0.12 | 14.00 | 63.23 | 22.77 | −0.19 | 0.25 | −0.06 | 0.5 |
| | | mM | | | | | | | | | | | | | |
| 11 | 5* | Prol Arg 100 | 5.8 | −0.1 | 0.67 | −0.04 | 98.11 | 0.01 | 14.06 | 63.70 | 22.25 | −0.52 | −0.71 | 1.22 | 2.45 |
| | | mM | | | | | | | | | | | | | |
| 12 | 5* | Prol PS80 | n/a | n/a | 0.66 | 0.02 | 97.97 | 0.01 | 13.45 | 62.23 | 24.33 | 0.19 | 0.05 | −0.25 | 0.49 |
| 13 | 5* | Prol Koll | n/a | n/a | 0.65 | 0.02 | 98.1 | −0.05 | 13.67 | 62.81 | 23.53 | −0.19 | −0.48 | 0.66 | 1.33 |
| 14 | 5* | Prol PPG | n/a | n/a | 0.58 | 0.06 | 98.03 | −0.12 | 14.00 | 62.82 | 23.19 | −0.67 | −0.39 | 1.04 | 2.1 |

*Protein concentration is method-specific and given in the method description.
Formulations 2′, 3′, 4′ and 6′ are repeats of formulations 2, 3, 4 and 6, respectively.

### Determination of Stability Under Freeze-Haw.

Stability under freeze-thaw was determined only for formulations comprising proline. Stability under freeze-thaw was assessed by method 9. Before and following three freeze-thaw cycles, we determined: purity by SE HPLC by method 13, charge variant profile in capillary by method 14, hydrodynamic radius by method 5; absolute change in charge variant profile was calculated by method 22. The results are shown in Table 17.

TABLE 17
Results of study of stability of anti-TRBV9 in various formulations under freeze-thaw.

| | Test formulation, | | Hydrodynamic | | SE HPLC | | | | Labchip charge variant profile, % | | | | | | |
|---|---|---|---|---|---|---|---|---|---|---|---|---|---|---|---|
| | mg/ml | | radius, nm | | Aggregates, % | | Monomer, % | | | | | | | | Δ |
| | | For- | | Δ fr- | | Δ fr- | | Δ fr- | | | | | | | abs. |
| | Pro- | mulation | | th 3 | | th 3 | | th 3 | Alc. | Base | Acid. | Alc. | Base | Acid. | fr-th 3 |
| No. | tein | designation | IC | cycle | IC | cycle | IC | cycle | IC | | | Δ fr-th 3 cycle | | | cycle |
| 6′ | 5* | Prol Gly | 4.3 | −0.3 | 0.67 | 0.01 | 98.04 | −0.01 | 13.56 | 63.22 | 23.23 | 0.15 | 0.72 | −0.87 | 1.74 |
| | | 100 mM | | | | | | | | | | | | | |
| 8 | 5* | Prol 30 | 1.6 | −0.4 | 0.71 | 0.03 | 98.01 | 0.01 | 11.92 | 64.99 | 23.10 | 1.34 | −1.91 | 0.57 | 3.82 |
| 9 | 5* | Prol 27 | 4.6 | −0.4 | 0.68 | 0.02 | 98.09 | −0.01 | 14.90 | 63.84 | 21.26 | −1.23 | −0.70 | 1.93 | 3.86 |
| 10 | 5* | Prol Meth | 4.4 | 1.1 | 0.78 | 0.02 | 98.18 | −0.23 | 14.00 | 63.23 | 22.77 | −0.17 | 0.46 | −0.29 | 0.92 |
| | | 10 mM | | | | | | | | | | | | | |
| 11 | 5* | Prol Arg | 5.8 | 0.6 | 0.67 | 0.02 | 98.11 | −0.06 | 14.06 | 63.70 | 22.25 | −0.57 | −0.88 | 1.44 | 2.89 |
| | | 100 mM | | | | | | | | | | | | | |
| 12 | 5 | Prol PS80 | n/a | n/a | 0.66 | 0.05 | 97.97 | 0.03 | 13.45 | 62.23 | 24.33 | −0.10 | −0.30 | 0.39 | 0.79 |
| 13 | 5 | Prol Koll | n/a | n/a | 0.65 | 0.04 | 98.1 | −0.09 | 13.67 | 62.81 | 23.53 | −0.35 | −0.77 | 1.11 | 2.23 |
| 14 | 5* | Prol PPG | n/a | n/a | 0.58 | 0.08 | 98.03 | −0.17 | 14.00 | 62.82 | 23.19 | −0.54 | −0.97 | 1.50 | 3.01 |

*Protein concentration is method-specific and given in the method description.

The given formulations showed excellent results in terms of colloidal and chemical stability: a slight change in the monomer content and the charge variant profile was revealed under thermal stress (SE HPLC, Labchip). Further, the given compositions showed high melting point and aggregation temperature, which fact indicates high stability of the protein under thermal stress. The given formulations also have a high concentration capacity.

### Example 5. Determination of Critical Quantitative Factors of Formulation and Formulation Optimization

The present study employs histidine buffer system as the basis of the pharmaceutical composition. The study was conducted in the fractional 3-factor experiment design with to levels. Protein concentration (from 10 to 50), pH (from 5.7 to 6.9), osmotic agent concentration (from 19 to 35) were studied as quantitative factors.

As part of the study, we assessed the effect of the test factors on colloidal and conformational stability of the protein. Aggregation temperature, melting point, diffusion interaction parameter, changes in purity and in acid-base profile following thermal stress, shaking, acid and alkaline hydrolysis, several freeze-thaw cycles, oxidation were determined as response.

Test formulations are shown in Table 18.

Table 19 shows the formulations of 5 mM histidine buffer for various pH involved in the experiment.

TABLE 18
Test formulations

| | | Protein | | | |
|---|---|---|---|---|---|
| | | C, | Buffer | | Proline C, |
| No. | Designation | mg/ml | solution | pH | mg/ml |
| 1 | 10 mg/ml His 5.7 + | 10 | 5 mM His | 5.7 | 35 |
| | 35 Prol | | | | |
| 2 | 30 mg/ml His 6.3 + | 30 | 5 mM His | 6.3 | 27 |
| | 27 Prol | | | | |
| 3 | 10 mg/ml His 6.9 + | 10 | 5 mM His | 6.9 | 19 |
| | 19 Prol | | | | |
| 4 | 50 mg/ml His 6.9 + | 50 | 5 mM His | 6.9 | 35 |
| | 35 Prol | | | | |
| 5 | 50 mg/ml His 5.7 + | 50 | 5 mM His | 5.7 | 19 |
| | 19 Prol | | | | |

TABLE 19
Formulations of buffer solutions

| Designation | Formulation | |
|---|---|---|
| 5 mM His, pH | L-histidine | 0.259 mg/ml |
| 5.7 | L-histidine hydrochloride | 0.698 mg/ml |
| | monohydrate | |
| 5 mM His, pH | L-histidine | 0.517 mg/ml |
| 6.3 | L-histidine hydrochloride | 0.35 mg/ml |
| | monohydrate | |
| 5 mM His, pH | L-histidine | 0.689 mg/ml |
| 6.9 | L-histidine hydrochloride | 0.117 mg/ml |
| | monohydrate | |

### Study of Stability Prediction Indicators.

Melting point and denaturation initiation point were determined by method 4, aggregation point was determined by method 3. The diffusion interaction parameter was determined by method 6, pH was measured by method 20. The results are shown in Table 20.

TABLE 20
Results of study of stability of anti-TRBV9 in various formulations

| | Test formulation, mg/ml | | pH of protein | DSF | | DLS | |
|---|---|---|---|---|---|---|---|
| No. | Protein | Formulation designation | solution | Tonset, ° C. | Tm, ° C. | Tag, ° C. | Kd |
| 1 | 10 | 10 mg/ml His 5.7 + 35 Prol | 5.75 | 53.8 | 66.1 | 65.27 | 5.29E−02 |
| 2 | 30 | 30 mg/ml His 6.3 + 27 Prol | 6.40 | 52.3 | 66.4 | 62.19 | 8.22E−02 |
| 3 | 10 | 10 mg/ml His 6.9 + 19 Prol | 6.75 | 53.5 | 65.8 | 62.33 | 9.98E−02 |
| 4 | 50 | 50 mg/ml His 6.9 + 35 Prol | 7.20 | 52.3 | 66.1 | 58.45 | 1.06E−01 |
| 5 | 50 | 50 mg/ml His 5.7 + 19 Prol | 5.86 | 53.5 | 65.8 | 61.86 | 6.75E−02 |

### Determination of Thermal Stability.

Thermal stability was assessed by method 7. Before and following thermal stress, we determined: purity by SE HPLC by method 13, purities by CE in non-reducing conditions by method 16, charge variant profile by method 18, hydrodynamic radius by method 5; absolute change in charge variant profile was calculated by method 22. The results are shown in Table 21.

TABLE 21
Results of study of stability of anti-TRBV9 in various formulations under thermal stress

| | Test formulation, | | Hydro- | | SE HPLC | | | | CE non-red. | | Charge variant profile (CZE), | | | | | | |
|---|---|---|---|---|---|---|---|---|---|---|---|---|---|---|---|---|---|
| | mg/ml | | dynamic | | Aggregates, % | | Monomer, % | | Monomer, % | | | | | | | | Δ |
| | | For- | radius, nm | | | Δ | | Δ | | Δ | | | | | | | abs |
| | Pro- | mulation | | TS50 | | TS50 | | TS50 | | TS50 | Alc. | Base | Acid. | Alc. | Base | Acid. | TS50 |
| No. | tein | designation | IC | 120H | IC | 120H | IC | 120H | IC | 120H | IC | | | Δ TS50 120H | | | 120H |
| 1 | 10 | 10 mg/ml His | 3.6 | 3.7 | 0.4 | 0.41 | 99.46 | 0.01 | 96.9 | −1.59 | 3.18 | 74.68 | 22.14 | 37.32 | −63.26 | 25.93 | 126.51 |
| | | 5.7 + 35 Prol | | | | | | | | | | | | | | | |
| 2 | 30 | 30 mg/ml His | 2.5 | 2.5 | 0.43 | 0.9 | 99.44 | 0.47 | 96.74 | −1.36 | 3.02 | 77.46 | 19.52 | −1.20 | −18.53 | 19.73 | 39.46 |
| | | 6.3 + 27 Prol | | | | | | | | | | | | | | | |
| 3 | 10 | 10 mg/ml His | 2.4 | 2.9 | 0.44 | 0.8 | 99.42 | 0.37 | 96.75 | −1.71 | 3.09 | 76.85 | 20.05 | −1.21 | −21.64 | 22.85 | 45.7 |
| | | 6.9 + 19 Prol | | | | | | | | | | | | | | | |
| 4 | 50 | 50 mg/ml His | 3.1 | 3.9 | 0.51 | 1.58 | 99.38 | 1.07 | 96.62 | −1.91 | 3.09 | 76.32 | 20.59 | 6.46 | −26.91 | 20.45 | 53.82 |
| | | 6.9 + 35 Prol | | | | | | | | | | | | | | | |
| 5 | 50 | 50 mg/ml His | 2.3 | 2.6 | 0.45 | 1.13 | 99.43 | 0.68 | 96.57 | −0.81 | 3.17 | 74.51 | 22.32 | 22.10 | −32.41 | 10.32 | 64.83 |
| | | 5.7 + 19 Prol | | | | | | | | | | | | | | | |

### Determination of Stability Under Shaking.

Stability under shaking was assessed by method 8. Before and following shaking, we determined: purity by SE HPLC by method 13, purity by electrophoresis in non-reducing conditions by method 16, charge variant profile in capillary by method 18, hydrodynamic radius by method 5; absolute change in charge variant profile was calculated by method 22. The results are shown in Table 22.

TABLE 22
Results of study of stability of anti-TRBV9 in various formulations under shaking

| | Test formulation, | | Hydro- | | SE HPLC | | | | CE non-red. | | | | | | | | |
|---|---|---|---|---|---|---|---|---|---|---|---|---|---|---|---|---|---|
| | mg/ml | | dynamic | | Aggregates, | | Monomer, | | Monomer, | | | | | | | | |
| | | For- | radius, | | % | | % | | % | | Charge variant profile (CZE), % | | | | | | |
| | | mulation | nm | | | Δ | | Δ | | Δ | | | | | | | Δ abs |
| | Pro- | desig- | | SH800 | | SH800 | | SH800 | | SH800 | Alc. | Base | Acid. | Alc. | Base | Acid. | SH800 |
| No. | tein | nation | IC | 120H | IC | 120H | IC | 120H | IC | 120H | IC | | | Δ SH800 120H | | | 120H |
| 1 | 10 | 10 mg/ml | 3.6 | 3.5 | 0.4 | 0.39 | 99.46 | −0.01 | 96.9 | 0.46 | 3.18 | 74.68 | 22.14 | −0.15 | −1.07 | 1.22 | 2.44 |
| | | His 5.7 + | | | | | | | | | | | | | | | |
| | | 35 Prol | | | | | | | | | | | | | | | |
| 2 | 30 | 30 mg/ml | 2.5 | 2.3 | 0.43 | 0.43 | 99.44 | 0 | 96.74 | 0.46 | 3.02 | 77.46 | 19.52 | 0.07 | −4.51 | 4.44 | 9.02 |
| | | His 6.3 + | | | | | | | | | | | | | | | |
| | | 27 Prol | | | | | | | | | | | | | | | |
| 3 | 10 | 10 mg/ml | 2.4 | 2.3 | 0.44 | 0.39 | 99.42 | −0.05 | 96.75 | 0.46 | 3.09 | 76.85 | 20.05 | −0.14 | −3.60 | 3.75 | 7.49 |
| | | His 6.9 + | | | | | | | | | | | | | | | |
| | | 19 Prol | | | | | | | | | | | | | | | |
| 4 | 50 | 50 mg/ml | 3.1 | 2.8 | 0.51 | 0.46 | 99.38 | −0.04 | 96.62 | 0.39 | 3.09 | 76.32 | 20.59 | −0.06 | −1.75 | 1.81 | 3.62 |
| | | His 6.9 + | | | | | | | | | | | | | | | |
| | | 35 Prol | | | | | | | | | | | | | | | |
| 5 | 50 | 50 mg/ml | 2.3 | 2.3 | 0.45 | 0.42 | 99.43 | −0.03 | 96.57 | 0.47 | 3.17 | 74.51 | 22.32 | −0.38 | 1.91 | −1.53 | 3.82 |
| | | His 5.7 + | | | | | | | | | | | | | | | |
| | | 19 Prol | | | | | | | | | | | | | | | |

### Determination of Stability Under Basic Hydrolysis.

Stability under basic hydrolysis was assessed by method 11. Before and following hydrolysis, we determined: purity by SE HPLC by method 13, purity by electrophoresis in non-reducing conditions by method 18, charge variant profile in capillary by method 14, hydrodynamic radius by method 5; absolute change in charge variant profile was calculated by method 22. The results are shown in Table 23.

TABLE 23
Results of study of stability of anti-TRBV9 in various formulations under basic hydrolysis

| | Test formulation, | | Hydro- | | SE HPLC | | | | CE non-red. | | Charge variant profile (CZE), % | | | | | | |
|---|---|---|---|---|---|---|---|---|---|---|---|---|---|---|---|---|---|
| | mg/ml | | dynamic | | Aggregates, | | Monomer, | | Monomer, | | | | | | | | Δ |
| | | For- | radius, nm | | % | | % | | % | | | | | | | | abs |
| | | mulation | | Basic | | Δ | | Δ | | Δ | | | | | | | Basic |
| | Pro- | desig- | | 9.0 | | Basic | | Basic | | Basic | Alc. | Base | Acid. | Alc. | Base | Acid. | 9.0 |
| No. | tein | nation | IC | 1H | IC | 9.0 1H | IC | 9.0 1H | IC | 9.0 1H | IC | | | Δ Basic 9.0 1H | | | 1H |
| 1 | 10 | 10 mg/ml | 3.5 | 5.8 | 0.46 | 0.82 | 99.47 | 0.36 | 97.32 | 0.35 | 7.06 | 76.36 | 16.58 | 0.12 | −0.69 | 0.57 | 1.38 |
| | | His 5.7 + | | | | | | | | | | | | | | | |
| | | 35 Prol | | | | | | | | | | | | | | | |
| 2 | 30 | 30 mg/ml | 2.4 | 5.2 | 0.45 | 1.47 | 99.47 | 1.02 | 96.78 | 0.32 | 7.00 | 75.78 | 17.21 | −1.52 | −0.56 | 2.09 | 4.17 |
| | | His 6.3 + | | | | | | | | | | | | | | | |
| | | 27 Prol | | | | | | | | | | | | | | | |
| 3 | 10 | 10 mg/ml | 2.4 | n/a | 0.44 | 2.15 | 99.5 | 1.71 | 96.92 | 0.36 | 6.11 | 76.29 | 17.59 | −1.67 | −5.26 | 6.94 | 13.87 |
| | | His 6.9 + | | | | | | | | | | | | | | | |
| | | 19 Prol | | | | | | | | | | | | | | | |
| 4 | 50 | 50 mg/ml | 3.3 | n/a | 0.52 | 2.82 | 99.41 | 2.31 | 96.89 | 0.76 | 6.45 | 75.81 | 17.73 | −3.05 | 1.30 | 1.76 | 6.11 |
| | | His 6.9 + | | | | | | | | | | | | | | | |
| | | 35 Prol | | | | | | | | | | | | | | | |
| 5 | 50 | 50 mg/ml | 2.4 | 4.5 | 0.46 | 2.81 | 99.48 | 2.35 | 97 | 0.35 | 7.19 | 75.94 | 16.87 | 0.50 | −0.11 | −0.39 | 1.0 |
| | | His 5.7 + | | | | | | | | | | | | | | | |
| | | 19 Prol | | | | | | | | | | | | | | | |

### Determination of Stability Under Acid Hydrolysis.

Stability under basic hydrolysis was assessed by method 10. Before and following hydrolysis, we determined: purity by SE HPLC by method 13, purity by electrophoresis in non-reducing conditions by method 16, charge variant profile in capillary by method 18, hydrodynamic radius by method 5; absolute change in charge variant profile was calculated by method 22. The results are shown in Table 24.

TABLE 24
Results of study of stability of anti-TRBV9 in various formulations under acid hydrolysis

| | Test formulation, | | | | SE HPLC | | | | CE non-red. | | | | | | | | |
|---|---|---|---|---|---|---|---|---|---|---|---|---|---|---|---|---|---|
| | mg/ml | | Hydro- | | Aggregates, | | Monomer, | | Monomer, | | Charge variant profile (CZE) | | | | | | |
| | | For- | dynamic | | % | | % | | % | | | | | | | | Δ |
| | | mulation | radius, nm | | | Δ | | Δ | | Δ | | | | | | | abs |
| | Pro- | desig- | | Acid | | Acid | | Acid | | Acid | Alc. | Base | Acid. | Alc. | Base | Acid. | Acid |
| No. | tein | nation | IC | 4.0 1H | IC | 4.0 1H | IC | 4.0 1H | IC | 4.0 1H | IC | | | Δ Acid 4.0 1H | | | 4.0 1H |
| 1 | 10 | 10 mg/ml | 3.5 | 6.2 | 0.46 | 0.54 | 99.47 | 0.08 | 97.32 | 0.38 | 7.06 | 76.36 | 16.58 | −0.78 | 0.98 | −0.2 | 1.96 |
| | | His 5.7 + | | | | | | | | | | | | | | | |
| | | 35 Prol | | | | | | | | | | | | | | | |
| 2 | 30 | 30 mg/ml | 2.5 | 6.8 | 0.47 | 0.75 | 99.46 | 0.28 | 96.98 | 0.34 | 6.58 | 76.56 | 16.86 | 0.04 | −0.09 | 0.06 | 0.19 |
| | | His 6.3 + | | | | | | | | | | | | | | | |
| | | 27 Prol | | | | | | | | | | | | | | | |
| 3 | 10 | 10 mg/ml | 2.4 | 6.2 | 0.44 | 0.92 | 99.5 | 0.48 | 96.92 | 0.33 | 6.11 | 76.29 | 17.59 | 0.03 | 0.74 | −0.76 | 1.53 |
| | | His 6.9 + | | | | | | | | | | | | | | | |
| | | 19 Prol | | | | | | | | | | | | | | | |
| 4 | 50 | 50 mg/ml | 3.3 | n/a | 0.52 | 1.83 | 99.41 | 1.31 | 96.89 | 0.37 | 6.45 | 75.81 | 17.73 | −0.27 | 0.6 | −0.32 | 1.19 |
| | | His 6.9 + | | | | | | | | | | | | | | | |
| | | 35 Prol | | | | | | | | | | | | | | | |
| 5 | 50 | 50 mg/ml | 2.4 | 6.1 | 0.46 | 1.08 | 99.48 | 0.63 | 97 | 0.34 | 7.19 | 75.94 | 16.87 | −0.69 | 1.4 | −0.71 | 2.8 |
| | | His 5.7 + | | | | | | | | | | | | | | | |
| | | 19 Prol | | | | | | | | | | | | | | | |

### Determination of Stability Under Freeze-Thaw.

Stability under basic hydrolysis was assessed by method 9. Before and following hydrolysis, we we determined: purity by SE HPLC by method 13, charge variant profile in capillary by method 18, hydrodynamic radius by method 5; absolute change in charge variant profile was calculated by method 22. The results are shown in Table 25.

TABLE 25
Results of study of stability of anti-TRBV9 in various formulations under five freeze-thaw cycles

| | | | Hydro- | | | | | | | | Charge variant profile (CZE) | | | | | | |
|---|---|---|---|---|---|---|---|---|---|---|---|---|---|---|---|---|---|
| | Test formulation, | | dynamic | | SE HPLC | | | | CE non-red. | | | | | | | | Δ |
| | mg/ml | | radius, nm | | Aggregates, % | | Monomer, % | | Monomer % | | | | | | | | abs |
| | Pro- | Formulation | | FT | | Δ FT | | Δ FT | | Δ FT | Alc. | Base | Acid. | Alc. | Base | Acid. | FT |
| No. | tein | designation | IC | 5C | IC | 5C | IC | 5C | IC | 5C | IC | | | FT 5C | | | 5C |
| 1 | 10 | 10 mg/ml | 3.6 | 3.2 | 0.4 | 0.41 | 99.46 | 0 | 96.9 | 0.47 | 3.18 | 74.68 | 22.14 | −0.48 | 2.03 | −1.54 | 4.05 |
| | | His 5.7 + | | | | | | | | | | | | | | | |
| | | 35 Prol | | | | | | | | | | | | | | | |
| 2 | 30 | 30 mg/ml | 2.5 | 2.2 | 0.43 | 0.45 | 99.44 | 0.02 | 96.74 | 0.41 | 3.02 | 77.46 | 19.52 | 0.3 | −1.29 | 1.59 | 3.18 |
| | | His 6.3 + | | | | | | | | | | | | | | | |
| | | 27 Prol | | | | | | | | | | | | | | | |
| 3 | 10 | 10 mg/ml | 2.4 | 2.2 | 0.44 | 0.45 | 99.42 | 0.01 | 96.75 | 0.44 | 3.09 | 76.85 | 20.05 | −0.27 | −2.13 | 2.4 | 4.8 |
| | | His 6.9 + | | | | | | | | | | | | | | | |
| | | 19 Prol | | | | | | | | | | | | | | | |
| 4 | 50 | 50 mg/ml | 3.1 | 2.8 | 0.51 | 0.49 | 99.38 | −0.01 | 96.62 | 0.47 | 3.09 | 76.32 | 20.59 | −0.34 | −0.97 | 1.3 | 2.61 |
| | | His 6.9 + | | | | | | | | | | | | | | | |
| | | 35 Prol | | | | | | | | | | | | | | | |
| 5 | 50 | 50 mg/ml | 2.3 | 2.4 | 0.45 | 0.44 | 99.43 | −0.01 | 96.57 | 0.46 | 3.17 | 74.51 | 22.32 | −0.4 | 1.8 | −1.4 | 3.6 |
| | | His 5.7 + | | | | | | | | | | | | | | | |
| | | 19 Prol | | | | | | | | | | | | | | | |

### Determination of Stability Under Oxidation.

Stability under oxidation was assessed by method 21. Before and following oxidation, we determined: 0 purity by SE HPLC by method 13, purity by electrophoresis in non-reducing conditions by method 16, charge variant profile in capillary by method 18, hydrodynamic radius by method 5; absolute change in charge variant profile was calculated by method 22. The results are shown in Table 26.

TABLE 26
Results of study of stability of anti-TRBV9 in various formulations under oxidation with H₂O₂

| | | | Hydro- | | SE HPLC | | | | CE non-red. | | Charge variant profile (CZE) | | | | | | |
|---|---|---|---|---|---|---|---|---|---|---|---|---|---|---|---|---|---|
| | Test formulation, | | dynamic | | Aggregates, % | | Monomer, % | | Monomer % | | | | | | | | Δ |
| | mg/ml | | radius, nm | | | Δ | | Δ | | Δ | | | | | | | abs |
| | Pro- | Formulation | | Oxid | | Oxid | | Oxid | | Oxid | Alc. | Base | Acid. | Alc. | Base | Acid. | Oxid |
| No. | tein | designation | IC | 0.1% | IC | 0.1% | IC | 0.1% | IC | 0.1% | IC | | | Δ Oxid 0.1% | | | 0.1% |
| 1 | 10 | 10 mg/ml His | 3.6 | 3.5 | 0.46 | −0.01 | 99.47 | 0.04 | 97.32 | −1.11 | 7.06 | 76.36 | 16.58 | −1.24 | −1.03 | 2.27 | 4.54 |
| | | 5.7 + 35 Prol | | | | | | | | | | | | | | | |
| 2 | 30 | 30 mg/ml His | 2.5 | 2.4 | 0.47 | 0.02 | 99.46 | −0.01 | 96.98 | −1.03 | 6.58 | 76.56 | 16.86 | −0.80 | −1.90 | 2.70 | 5.4 |
| | | 6.3 + 27 Prol | | | | | | | | | | | | | | | |
| 3 | 10 | 10 mg/ml His | 2.4 | 2.6 | 0.44 | 0.01 | 99.5 | −0.02 | 96.92 | −0.72 | 6.11 | 76.29 | 17.59 | −0.49 | −2.98 | 3.49 | 6.96 |
| | | 6.9 + 19 Prol | | | | | | | | | | | | | | | |
| 4 | 50 | 50 mg/ml His | 3.1 | 2.9 | 0.52 | −0.04 | 99.41 | 0.05 | 96.89 | −0.78 | 6.45 | 75.81 | 17.73 | −0.81 | −2.88 | 3.70 | 7.39 |
| | | 6.9 + 35 Prol | | | | | | | | | | | | | | | |
| 5 | 50 | 50 mg/ml His | 2.3 | 2.5 | 0.46 | −0.01 | 99.48 | 0.02 | 97 | −1.02 | 7.19 | 75.94 | 16.87 | −0.99 | −1.17 | 2.16 | 4.32 |
| | | 5.7 + 19 Prol | | | | | | | | | | | | | | | |

The given formulations showed excellent values of melting point, aggregation temperature, hydrodynamic radius, as well as the value of the diffusion interaction parameter, which fact indicates high stability under thermal and other types of stress. The given formulations also demonstrated a positive effect on colloidal and chemical stability: a slight change in the monomer content (SE HPLC, CE non-red.) under oxidation, basic and acid hydrolysis and freeze-thaw.

### Example 6. Selection of Additional Osmotic Agents and Stabilizers

The present study employs histidine buffer system (5 mM histidine buffer, pH 6.3) as the basis of the pharmaceutical composition. Excipients suitable for parenteral administration were studied to be used as stabilizers and surfactants.

As part of the study, we assessed the effect of each of the test osmotic agents, stabilizers and surfactants on colloidal and conformational stability of the protein. Aggregation temperature, melting point, diffusion interaction parameter, changes in purity and in acid-base profile following thermal stress, shaking, several freeze-thaw cycles were determined as response. Further, the hydrodynamic radius and purity (SE HPLC) before and following storage for 24 hours at 2-8° C. were evaluated in infusion solutions of the given formulations.

Test formulations are shown in Table 27.

TABLE 27
Test formulations

| No. | Designation | Formulation | |
|---|---|---|---|
| 1 | Prol 27 (5 mM | anti-TRBV9 | 5 mg/ml |
| | His. pH 6.3) | L-histidine | 0.517 mg/ml |
| | | L-histidine hydrochloride | 0.35 mg /ml |
| | | monohydrate | |
| | | L-proline | 27 mg/ml |
| | | Water for injection | to 1 ml |
| 2 | Prol 27 + 1 | anti-TRBV9 | 5 mg/ml |
| | PS80 (5 mM His. | L-histidine | 0.517 mg/ml |
| | pH 6.3) | L-histidine hydrochloride | 0.35 mg/ml |
| | | monohydrate | |
| | | L-proline | 27 mg/ml |
| | | Polysorbate 80 | 1 mg/ml |
| | | Water for injection | to 1 ml |
| 3 | Prol + 1 | anti-TRBV9 | 5 mg/ml |
| | PLXM188 (5 mM | L-histidine | 0.517 mg/ml |
| | His. pH 6.3) | L-histidine hydrochloride | 0.35 mg/ml |
| | | monohydrate | |
| | | L-proline | 27 mg/ml |
| | | Poloxamer 188 | 1 mg/ml |
| | | Water for injection | to 1 ml |
| 4 | Tre 100 (5 mM | anti-TRBV9 | 5 mg/ml |
| | His. pH 6.3) | L-histidine | 0.517 mg/ml |
| | | L-histidine hydrochloride | 0.35 mg/ml |
| | | monohydrate | |
| | | Trehalose | 100 mg/ml |
| | | Water for injection to 1 ml | |
| 5 | Tre + 1 PS80 (5 | anti-TRBV9 | 5 mg/ml |
| | mM His. pH 6.3) | L-histidine | 0.517 mg/ml |
| | | L-histidine hydrochloride | 0.35 mg/ml |
| | | monohydrate | |
| | | Trehalose | 100 mg/ml |
| | | Polysorbate 80 | 1 mg/ml |
| | | Water for injection | to 1 ml |
| 6 | Tre + 1 PLXM188 | anti-TRBV9 | 5 mg/ml |
| | (5 mM His. pH | L-histidine | 0.517 mg/ml |
| | 6.3) | L-histidine hydrochloride | 0.35 mg/ml |
| | | monohydrate | |
| | | Trehalose | 100 mg/ml |
| | | Poloxamer 188 | 1 mg/ml |
| | | Water for injection | to 1 ml |
| 7 | NaCl 9 (5 mM | anti-TRBV9 | 5 mg/ml |
| | His. pH 6.3) | L-histidine | 0.517 mg/ml |
| | | L-histidine hydrochloride | 0.35 mg/ml |
| | | monohydrate | |
| | | NaCl | 9 mg/ml |
| | | Water for injection | to 1 ml |
| 8 | NaCl 9 + 1 PS80 | anti-TRBV9 | 5 mg/ml |
| | (5 mM His. pH | L-histidine | 0.517 mg/ml |
| | 6.3) | L-histidine hydrochloride | 0.35 mg/ml |
| | | monohydrate | |
| | | NaCl | 9 mg/ml |
| | | Polysorbate 80 | 1 mg/ml |
| | | Water for injection | to 1 ml |
| 9 | NaCl 9 + 1 | anti-TRBV9 | 5 mg/ml |
| | PLXM188 (5 mM | L-histidine | 0.517 mg/ml |
| | His. pH 6.3) | L-histidine hydrochloride | 0.35 mg/ml |
| | | monohydrate | |
| | | NaCl | 9 mg/ml |
| | | Poloxamer 188 | 1 mg/ml |
| | | Water for injection | to 1 ml |

### Study of Stability Prediction Indicators.

Those formulations comprising surfactants did not undergo heating-related rests, such as determination of denaturation onset temperature and melting point, aggregation temperature, which is due to degradation of surfactants under heating and, as a result, non-interpretable results. Also, they did not undergo direct concentrating as micelle formation caused by surfactants may affect the concentrating process.

Results of the determination of aggregation temperature by method 3, denaturation onset temperature and melting point by method 4, diffusion interaction parameter by method 6 and direct concentrating by method 1 are shown in Table 28.

TABLE 28
Results of study of physico-chemical characteristics of anti-TRBV9 in various formulations.

| | DSF | | | | DLS | | Direct concentrating | | | | | |
|---|---|---|---|---|---|---|---|---|---|---|---|---|
| | Formulation | Tonset, | Tm1, | Tm2, | Tag, | | C fin., | V fin., | Yield, | Cfin./ | C η, | η cp, |
| No. | designation | ° C. | ° C. | ° C. | ° C. | kd | mg/ml | ml | % | Cin. | mg/ml | mPa*s |
| 1 | Prol 27 | 52.6 | 66.1 | n/a | 65.66 | 5.52E−02 | 59.3 | 0.62 | 75 | 12.1 | 54.1 | 2.02 |
| 4 | Tre 100 | 52.9 | 67.3 | n/a | 65.81 | 5.66E−02 | 77.2 | 0.57 | 89.8 | 15.8 | 54.4 | 2.26 |
| 7 | NaCl 9 | 51.7 | 64.6 | 69.1 | 61.05 | −3.35E−03 | 54.1 | 0.875 | 94.7 | 10.8 | 54.1 | 1.76 |

*Concentration of a sample for which viscosity was determined.

### Determination of Thermal Stability.

Thermal stability was also not studied for surfactant formulations due to degradation thereof under heating.

Thermal stability was assessed by method 7. Before and following thermal stress, we determined: purity by SE HPLC by method 13, charge variant profile by method 15, hydrodynamic radius by method 5; absolute change in charge variant profile was calculated by method 22. The results are shown in Table 29.

TABLE 29
Results of study of stability of anti-TRBV9 in various formulations under thermal stress

| | | | | | SE HPLC | | | | | | | | | | |
|---|---|---|---|---|---|---|---|---|---|---|---|---|---|---|---|
| | Test formulation, | | Hydrodynamic | | Aggregates, % | | Monomer, % | | Charge variant profile, % | | | | | | |
| | mg/ml | | radius, nm | | | Δ | | Δ | | | | | | | Δ abs |
| | Pro- | Formulation | | TS50 | | TS50 | | TS50 | Alc. | Base | Acid. | Alc. | Base | Acid. | TS50 |
| No. | tein | designation | IC | 96H | IC | 96H | IC | 96H | IC | | | Δ TS50 96H | | | 96H |
| 1 | 4.9 | Prol 27 | 3.8 | 3.9 | 0.46 | 0.12 | 99.42 | −0.59 | 8.51 | 63.96 | 27.53 | 12.85 | −16.11 | 3.26 | 32.22 |
| 4 | 4.9 | Tre 100 | 3.8 | 4 | 0.47 | 0.09 | 99.42 | −0.52 | 8.16 | 63.68 | 28.16 | 13.43 | −15.81 | 2.38 | 31.62 |
| 7 | 5 | NaCl 9 | 5.9 | 6.8 | 0.45 | −0.45 | 99.43 | −1.02 | 9.99 | 63.68 | 26.33 | 11.18 | −14.9 | 3.72 | 29.8 |

### Determination of Stability Under Shaking.

Stability under shaking was assessed by method 8. Before and following shaking, we determined: purity by SE HPLC by method 13, charge variant profile by method 15, hydrodynamic radius by method 5; absolute change in charge variant profile was calculated by method 22. The results are shown in Table 30.

TABLE 30
Results of study of stability of anti-TRBV9 in various formulations under shaking

| | | | | | SE HPLC | | | | Charge variant profile, % | | | | | | |
|---|---|---|---|---|---|---|---|---|---|---|---|---|---|---|---|
| | Test formulation, | | Hydrodynamic | | Aggregates, % | | Monomer, % | | | | | | | | Δ |
| | mg/ml | | radius, nm | | | Δ | | Δ | | | | | | | abs |
| | Pro- | Formulation | | SH800 | | SH800 | | SH800 | Alc. | Base | Acid. | Alc. | Base | Acid. | SH800 |
| No. | tein | designation | IC | 96H | IC | 96H | IC | 96H | IC | | | Δ SH800 96H | | | 96H |
| 1 | 4.9 | Prol 27 | 3.8 | 3.6 | 0.46 | −0.03 | 99.42 | 0.07 | 8.51 | 63.96 | 27.53 | 1.24 | −0.79 | −0.45 | 2.48 |
| 2 | 4.9 | Prol 27 + 1 | 3.8 | 3.7 | 0.55 | 0.2 | 99.34 | −0.18 | 8.39 | 63.76 | 25.85 | 1.38 | −0.84 | 1.47 | 3.69 |
| | | PS80 | | | | | | | | | | | | | |
| 3 | 4.9 | Prol 27 + 1 | 3.5 | 3.2 | 0.54 | −0.05 | 99.34 | 0.09 | 8.47 | 63.52 | 28.01 | 1.24 | −0.3 | −0.94 | 2.48 |
| | | PLXM188 | | | | | | | | | | | | | |
| 4 | 4.9 | Tre 100 | 3.8 | 3.7 | 0.47 | 0 | 99.42 | 0.03 | 8.16 | 63.68 | 28.16 | 1.63 | −0.57 | −1.06 | 3.26 |
| 5 | 4.9 | Tre 100 + 1 | 3.6 | 3.8 | 0.55 | 0.18 | 99.34 | 0.11 | 8.06 | 63.6 | 28.34 | 1.62 | −0.77 | −0.85 | 3.24 |
| | | PS80 | | | | | | | | | | | | | |
| 6 | 4.9 | Tre 100 + 1 | 4 | 3.8 | 0.54 | −0.03 | 99.35 | −0.17 | 9.92 | 63.66 | 26.42 | −0.19 | −0.35 | 0.55 | 1.09 |
| | | PLXM188 | | | | | | | | | | | | | |
| 7 | 5 | NaCl 9 | 5.9 | 5.7 | 0.45 | 0.02 | 99.43 | −0.02 | 9.99 | 63.68 | 26.33 | −0.3 | −0.05 | 0.35 | 0.7 |
| 8 | 5 | NaCl 9 + 1 | 5.8 | 5.6 | 0.55 | 0.19 | 99.32 | 0.13 | 9.93 | 63.59 | 26.48 | −0.05 | −0.41 | 0.46 | 0.92 |
| | | PS80 | | | | | | | | | | | | | |
| 9 | 5 | NaCl 9 + 1 | 5.7 | 5.5 | 0.53 | −0.03 | 99.34 | −0.17 | 9.78 | 63.78 | 26.44 | −0.19 | −0.31 | 0.51 | 1.01 |
| | | PLXM188 | | | | | | | | | | | | | |

### Determination of Stability Under Freeze-Thaw.

Stability under freeze-thaw was assessed by method 9. Before and following five freeze-thaw cycles, we determined: purity by SE HPLC by method 13, charge variant profile by method 15, hydrodynamic radius by method 5; absolute change in charge variant profile was calculated by method 22. The results are shown in Table 31.

TABLE 31
Results of study of stability of anti-TRBV9 in various formulations under freeze-thaw

| | Test formulation, | | Hydrodynamic | | SE HPLC | | | | | | | | | | |
|---|---|---|---|---|---|---|---|---|---|---|---|---|---|---|---|
| | mg/ml | | radius, nm | | Aggregates, % | | Monomer, % | | Charge variant profile, % | | | | | | |
| | Pro- | Formulation | | Δ | | Δ | | Δ | Alc. | Base | Acid. | Alc. | Base | Acid. | Δ abs |
| No. | tein | designation | IC | FT5C | IC | FT5C | IC | FT5C | IC | | | Δ FT5C | | | Δ FT5C |
| 1 | 4.9 | Prol 27 | 3.8 | 3.7 | 0.46 | −0.04 | 99.42 | −0.22 | 8.51 | 63.96 | 27.53 | 1.13 | 0.10 | −1.22 | 2.45 |
| 2 | 4.9 | Prol 27 + 1 | 3.8 | 3.9 | 0.55 | −0.11 | 99.34 | −0.17 | 8.39 | 63.76 | 25.85 | −8.39 | 0.02 | 0.84 | 9.25 |
| | | PS80 | | | | | | | | | | | | | |
| 3 | 4.9 | Prol 27 + 1 | 3.5 | 3.6 | 0.54 | −0.11 | 99.34 | −0.18 | 8.47 | 63.52 | 28.01 | 1.17 | 0.54 | −1.7 | 3.41 |
| | | PLXM188 | | | | | | | | | | | | | |
| 4 | 4.9 | Tre 100 | 3.8 | 3.6 | 0.47 | −0.05 | 99.42 | −0.24 | 8.16 | 63.68 | 28.16 | 1.37 | 0.1 | −1.47 | 2.94 |
| 5 | 4.9 | Tre 100 + 1 | 3.6 | 3.8 | 0.55 | −0.12 | 99.34 | −0.2 | 8.06 | 63.6 | 28.34 | 1.39 | 0.23 | −1.62 | 3.24 |
| | | PS80 | | | | | | | | | | | | | |
| 6 | 4.9 | Tre 100 + 1 | 4 | 3.7 | 0.54 | −0.12 | 99.35 | −0.16 | 9.92 | 63.66 | 26.42 | −0.55 | 0.39 | 0.15 | 1.09 |
| | | PLXM188 | | | | | | | | | | | | | |
| 7 | 5 | NaCl 9 | 5.9 | 5.9 | 0.45 | −0.03 | 99.43 | −0.26 | 9.99 | 63.68 | 26.33 | −0.32 | −0.16 | 0.48 | 0.96 |
| 8 | 5 | NaCl 9 + 1 PS80 | 5.8 | 5.8 | 0.55 | −0.1 | 99.32 | −0.18 | 9.93 | 63.59 | 26.48 | −0.13 | −0.28 | 0.41 | 0.82 |
| 9 | 5 | NaCl 9 + 1 | 5.7 | 6.1 | 0.53 | −0.1 | 99.34 | −0.18 | 9.78 | 63.78 | 26.44 | −0.58 | −0.03 | 0.61 | 1.22 |
| | | PLXM188 | | | | | | | | | | | | | |

### Determination of Stability of Infusion Solutions of Resulting Formulation.

Infusion solutions were prepared according to method 23. Before and following storage of infusion solutions, purity was determined by SE HPLC by method 13, hydrodynamic radius was determined by method 5. The results are shown in Table 32.

TABLE 32
Results of
study of stability of anti-TRBV9 in
various formulations under dilution with infusion solution

| | | | | | SE HPLC | | | |
|---|---|---|---|---|---|---|---|---|
| | Test formulation, mg/ml | | Hydrodynamic radius, nm | | Aggregates, % | | Monomer, % | |
| | | Formulation | | Δ 24H | | Δ 24H | | Δ 24H |
| No. | Protein | designation | IC | 5C | IC | 5C | IC | 5C |
| 1 | 0.5 | Prol 27 | 3.8 | 5.5 | 0.34 | −0.04 | 99.52 | −0.19 |
| 2 | 0.5 | Prol 27 + 1 PS80 | 3.8 | 5.1 | 0.48 | −0.09 | 99.37 | −0.17 |
| 3 | 0.5 | Prol 27 + 1 PLXM188 | 3.5 | 5.1 | 0.55 | −0.11 | 99.3 | −0.16 |
| 4 | 0.5 | Tre 100 | 3.8 | 5.3 | 0.49 | −0.06 | 99.35 | −0.2 |
| 5 | 0.5 | Tre 100 + 1 PS80 | 3.6 | 5.5 | 0.47 | −0.08 | 99.39 | −0.18 |
| 6 | 0.5 | Tre 100 + 1 PLXM188 | 4 | 4.9 | 0.49 | −0.04 | 99.38 | −0.24 |
| 7 | 0.5 | NaCl 9 | 5.9 | 5.4 | 0.49 | −0.05 | 99.36 | −0.21 |
| 8 | 0.5 | NaCl 9 + 1 PS80 | 5.8 | 5.2 | 0.46 | −0.06 | 99.4 | −0.2 |
| 9 | 0.5 | NaCl 9 + 1 PLXM188 | 5.7 | 5.2 | 0.51 | −0.08 | 99.35 | −0.22 |

The given formulations showed high denaturation onset temperature and melting point, as well as aggregation temperature and diffusion interaction parameter, which fact indicates increased thermal stability. The given formulations demonstrated excellent colloidal stability under shaking, freeze-thaw and dilution with infusion solution; also, the given formulations showed a slight change in the monomer content followed by SE HPLC and excellent stability of charge variant profile followed by IE HPLC under all types of stress.

### Example 7. Confirmation of Stability Under Accelerated Ageing

The present study employs histidine buffer system (5 mM histidine buffer, pH 6.3) as the basis of the pharmaceutical composition, proline was used as an osmotic agent. As part of the study, we studied the effect of adding surfactants at two different concentrations as follows: 0.5 and 1.0 mg/ml, a sample without surfactants was used as a reference sample.

Before and following stress, changes in concentration, pH, purity and acid-base profile were determined.

Test formulations are shown in Table 33.

TABLE 33
Test formulations.

| No. | Designation | Formulation | |
|---|---|---|---|
| 1 | Formulation | L-histidine | 0.517 mg/ml |
| | 1* | L-histidine hydrochloride monohydrate | 0.350 mg/ml |
| | | L-proline | 27 mg/ml |
| | | Water for injection | to 1 ml |
| 2 | Formulation | L-histidine | 0.517 mg/ml |
| | 2* | L-histidine hydrochloride monohydrate | 0.350 mg/ml |
| | | L-proline | 27 mg/ml |
| | | Poloxamer 188 | 0.5 mg/ml |
| | | Water for injection | to 1 ml |
| 3 | Formulation | L-histidine | 0.517 mg/ml |
| | 3* | L-histidine hydrochloride monohydrate | 0.350 mg/ml |
| | | L-proline | 27 mg/ml |
| | | Poloxamer 188 | 1 mg/ml |
| | | Water for injection | to 1 ml |

* Protein concentrations of 25, 50, 100 mg/ml were studied

### Determination of Stability Under Accelerated Storage.

Accelerated stability was assessed by method 12. Before and following stress, the protein content was determined by method 2, pH by method 20, purity by SE HPLC by method 13, charge variant profile in capillary by method 14.

Results of the study are shown in Tables 34-36.

TABLE 34
Results of study of stability of formulation 1 under accelerated storage at +37° C. at
concentrations of 25, 50 and 100 mg/ml.

| Sample info | Indicator | C theor. mg/ml | IC | AS37 2W | AS37 4W | Δ AS37 4W |
|---|---|---|---|---|---|---|
| Formulation 1 | C, mg/ml | 25 | 26 | n/a | n/a | n/a |
| | | 50 | 49 | n/a | n/a | n/a |
| | | 100 | 103 | n/a | n/a | n/a |
| | pH | 25 | 6.25 | n/a | 6.29 | n/a |
| | | 50 | 6.36 | n/a | 6.36 | n/a |
| | | 100 | 6.41 | n/a | 6.42 | n/a |
| | SE HPLC | 25 | n/a | 0.92 | 1.074 | 0.154 |
| | (monomer | | n/a | 95.29 | 94.68 | −0.61 |
| | content, %) | 50 | n/a | 1.41 | 1.65 | 0.24 |
| | | | n/a | 95.09 | 94.41 | −0.68 |
| | | 100 | n/a | 2.24 | 2.63 | 0.39 |
| | | | n/a | 93.28 | 91.94 | −1.34 |
| | CE (monomer | 25 | n/a | 95.88 | 96.18 | 0.30 |
| | content, %) | 50 | n/a | 94.17 | 93.72 | −0.45 |
| | | 100 | n/a | 96.05 | 94.97 | −1.08 |
| | | 25 | n/a | 92.79 | 91.46 | −1.33 |
| | | 50 | n/a | 92.87 | 91.47 | −1.40 |
| | | 100 | n/a | 91.79 | 89.99 | −1.80 |
| | IE HPLC (top | 25 | 21.000 | 23.802 | 26.35 | 5.35 |
| | to bottom: | | 60.408 | 46.79 | 39.487 | −20.921 |
| | acidic form | | 18.592 | 29.408 | 34.163 | 15.571 |
| | contents, %; | Δ abs ABP | n/a | 27.236 | 41.842 | n/a |
| | basic form | 50 | 21.279 | 24.804 | 27.603 | 6.324 |
| | contents, %; | | 60.381 | 47.5 | 41.28 | −19.101 |
| | alkaline form | | 18.340 | 27.696 | 31.117 | 12.777 |
| | contents, %) | Δ abs ABP | n/a | 25.762 | 38.202 | n/a |
| | | 100 mg/ml | 21.021 | 25.105 | 28.03 | 7.009 |
| | | | 59.855 | 48.176 | 42.047 | −17.808 |
| | | | 19.125 | 26.719 | 29.923 | 10.798 |
| | | Δ abs ABP | n/a | 23.357 | 35.615 | n/a |

TABLE 35
Results of study of stability of formulation 2 under accelerated storage at +37° C. at
concentrations of 25, 50 and 100 mg/ml.

| | | C theor. | | | | |
|---|---|---|---|---|---|---|
| Sample info | Indicator | mg/ml | IC | AS37 2W | AS37 4W | Δ AS37 4W |
| Formulation 2 | C, mg/ml | 25 | 26 | n/a | n/a | n/a |
| | | 50 | 52 | n/a | n/a | n/a |
| | | 100 | 102 | n/a | n/a | n/a |
| | pH | 25 | 6.26 | n/a | 6.27 | n/a |
| | | 50 | 6.32 | n/a | 6.28 | n/a |
| | | 100 | 6.39 | n/a | 6.41 | n/a |
| | SE HPLC | 25 | n/a | 0.96 | 1.09 | 0.13 |
| | (monomer | | n/a | 95.20 | 94.37 | −0.83 |
| | content, %) | 50 | n/a | 2.23 | 2.96 | 0.73 |
| | | | n/a | 92.00 | 88.50 | −3.5 |
| | | 100 | n/a | 2.26 | 2.62 | 0.36 |
| | | | n/a | 92.86 | 91.56 | −1.3 |
| | CE (monomer | 25 | n/a | 95.75 | 96.15 | 0.40 |
| | content, %) | 50 | n/a | 95.76 | 93.82 | −1.94 |
| | | 100 | n/a | 96.12 | 94.56 | −1.56 |
| | | 25 | n/a | 92.12 | 91.60 | −0.52 |
| | | 50 | n/a | 91.21 | 88.58 | −2.63 |
| | | 100 | n/a | 92.68 | 90.63 | −2.05 |
| | IE HPLC (top | 25 | 20.77 | 24.027 | 26.096 | 5.326 |
| | to bottom: | | 60.671 | 46.962 | 39.783 | −20.888 |
| | acidic form | | 18.559 | 29.011 | 34.121 | 15.562 |
| | contents, %; | Δ abs ABP | n/a | 27.418 | 41.776 | n/a |
| | basic form | 50 | 21.319 | 28.324 | 31.717 | 10.398 |
| | contents, %; | | 60.588 | 44.942 | 37.527 | −23.061 |
| | alkaline form | | 18.093 | 26.734 | 30.756 | 12.663 |
| | contents, %) | Δ abs ABP | n/a | 31.292 | 46.122 | n/a |
| | | 100 | 20.497 | 25.168 | 28.016 | 7.519 |
| | | | 60.456 | 48.359 | 41.156 | −19.3 |
| | | | 19.047 | 26.473 | 30.828 | 11.781 |
| | | Δ abs ABP | n/a | 24.194 | 38.6 | n/a |

TABLE 36
Results of study of stability of formulation 3 under accelerated storage at +37° C. at
concentrations of 25, 50 and 100 mg/ml.

| | | C theor. | | | | |
|---|---|---|---|---|---|---|
| Sample info | Indicator | mg/ml | IC | AS37 2W | AS37 4W | Δ AS37 4W |
| Formulation 3 | C, mg/ml | 25 | 26 | n/a | n/a | n/a |
| | | 50 | 48 | n/a | n/a | n/a |
| | | 100 | 103 | n/a | n/a | n/a |
| | pH | 25 | 6.23 | n/a | 6.25 | n/a |
| | | 50 | 6.34 | n/a | 6.36 | n/a |
| | | 100 | 6.39 | n/a | 6.42 | n/a |
| | SE HPLC | 25 | n/a | 1.07 | 1.08 | 0.01 |
| | (monomer | | n/a | 94.68 | 94.14 | −0.54 |
| | content, %) | 50 | n/a | 1.40 | 1.63 | 0.23 |
| | | | n/a | 95.17 | 94.34 | −0.83 |
| | | 100 | n/a | 2.32 | 2.55 | 0.23 |
| | | | n/a | 92.94 | 91.74 | −1.2 |
| | CE (monomer | 25 | n/a | 94.76 | 95.08 | 0.32 |
| | content, %) | 50 | n/a | 96.82 | 94.89 | −1.93 |
| | | 100 | n/a | 95.51 | 94.23 | −1.28 |
| | | 25 | n/a | 92.93 | 91.65 | −1.28 |
| | | 50 | n/a | 93.56 | 92.83 | −0.73 |
| | | 100 | n/a | 92.50 | 91.20 | −1.30 |
| | IE HPLC (top | 25 | 21.103 | 24.18 | 26.234 | 5.131 |
| | to bottom: | | 60.051 | 46.5 | 39.455 | −20.596 |
| | acidic form | | 18.846 | 29.32 | 34.312 | 15.466 |
| | contents, %; | Δ abs ABP | n/a | 27.102 | 41.193 | n/a |
| | basic form | 50 | 21.405 | 24.722 | 27.078 | 5.673 |
| | contents, %; | | 60.41 | 48.117 | 41.129 | −19.281 |
| | alkaline form | | 18.184 | 27.161 | 31.792 | 13.608 |
| | contents, %) | Δ abs ABP | n/a | 24.587 | 38.562 | n/a |
| | | 100 | 20.922 | 25.443 | 27.657 | 6.735 |
| | | | 59.932 | 48.448 | 41.908 | −18.024 |
| | | | 19.146 | 26.109 | 30.435 | 11.289 |
| | | Δ abs ABP | n/a | 22.968 | 36.048 | n/a |

The given formulations demonstrated excellent colloidal stability in the study of stability under accelerated storage (T=37° C.), as well as a high monomer content followed by SE HPLC and stabilization of charge variant profile followed by IE HPLC.

1. A pharmaceutical composition of anti-TRBV9 antibody, comprising:
(i) anti-TRBV9 antibody;
(ii) histidine buffer or acetate buffer; and
(iii) water for injection.

2. The pharmaceutical composition as claimed in claim 1, wherein the anti-TRBV9 antibody comprises:
1) a heavy chain variable domain comprising:
(a) HCDR1 having the amino acid sequence of SEQ ID NO: 1,
(b) HCDR2 having the amino acid sequence of SEQ ID NO: 2, and
(c) HCDR3 having an amino acid sequence selected from the group consisting of SEQ ID NO: 3, SEQ ID NO: 4, SEQ ID NO: 5 and SEQ ID NO: 6; and
2) a light chain variable domain comprising:
(a) LCDR1 having the amino acid sequence of SEQ ID NO: 7,
(b) LCDR2 having the amino acid sequence of SEQ ID NO: 8, and
(c) LCDR3 having the amino acid sequence of SEQ ID NO: 9.

3-4. (canceled)

5. The pharmaceutical composition as claimed in claim 1, wherein the anti-TRBV9 antibody comprises
a heavy chain variable domain having the amino acid sequence of SEQ ID No: 14
and a light chain variable domain having the amino acid sequence of SEQ ID No: 17; or
a heavy chain variable domain having the amino acid sequence of SEQ ID No: 15
and a light chain variable domain having the amino acid sequence of SEQ ID No: 17.

6. The pharmaceutical composition as claimed in claim 1, wherein the anti-TRBV9 antibody comprises
a heavy chain having the amino acid sequence of SEQ ID No: 22 and a light chain having the amino acid sequence of SEQ ID No: 25; or
a heavy chain having the amino acid sequence of SEQ ID No: 23 and a light chain having the amino acid sequence of SEQ ID No: 25.

7. (canceled)

8. The pharmaceutical composition as claimed in claim 1, wherein the anti-TRBV9 antibody is present at a concentration of 0.5-225.0 mg/ml.

9-12. (canceled)

13. The pharmaceutical composition as claimed in claim 1, wherein the histidine buffer is a mixture of histidine and histidine hydrochloride monohydrate.

14-22. (canceled)

23. The pharmaceutical composition as claimed in claim 1, wherein the acetate buffer is a mixture of sodium acetate and acetic acid.

24-32. (canceled)

33. The pharmaceutical composition as claimed in claim 1, further comprising one or more osmotic agents.

34. (canceled)

35. The pharmaceutical composition as claimed in claim 33, wherein the osmotic agent is proline, sorbitol, trehalose or sodium chloride.

36-48. (canceled)

49. The pharmaceutical composition as claimed in claim 1, further comprising one or more stabilizers.

50-51. (canceled)

52. The pharmaceutical composition as claimed in claim 49, wherein the stabilizer is an amino acid or surfactant.

53-54. (canceled)

55. The pharmaceutical composition as claimed in claim 52, wherein the amino acid is glycine or methionine.

56-65. (canceled)

66. The pharmaceutical composition as claimed in claim 52, wherein the surfactant is poloxamer 188, polysorbate 80 or polypropylene glycol.

67-71. (canceled)

72. The pharmaceutical composition as claimed in claim 33, comprising:
(i) anti-TRBV9 antibody;
(ii) histidine buffer or acetate buffer;
(iii) osmotic agent being proline 14.0-32.0 mg/ml;
(iv) water for injection to 1 ml;
or
(i) anti-TRBV9 antibody;
(ii) histidine buffer or acetate buffer,
(iii) osmotic agent being sorbitol 35.0-65.0 mg/ml;
(iv) water for injection to 1 ml;
or
(i) anti-TRBV9 antibody;
(ii) histidine buffer or acetate buffer;
(iii) osmotic agent being trehalose 70-130.0 mg/ml;
(iv) water for injection to 1 ml;
or
(i) anti-TRBV9 antibody;
(ii) histidine buffer or acetate buffer;
(iii) osmotic agent being sodium chloride 6.0-11.5 mg/ml;
(iv) water for injection to 1 ml.

73. The pharmaceutical composition as claimed in claim 33, comprising:
(i) anti-TRBV9 antibody;
(ii) histidine buffer being a mixture of
histidine 0.4-1.0 mg/ml;
histidine hydrochloride monohydrate 0.08-4.2 mg/ml;
(iii) osmotic agent being proline 14.0-32.0 mg/ml;
(iv) water for injection to 1 ml;
or
(i) anti-TRBV9 antibody;
(ii) histidine buffer being a mixture of
histidine 0.4-1.0 mg/ml;
histidine hydrochloride monohydrate 0.08-4.2 mg/ml;
(iii) osmotic agent being sorbitol 35.0-65.0 mg/ml;
(iv) water for injection to 1 ml;
or
(i) anti-TRBV9 antibody;
(ii) histidine buffer being a mixture of
histidine 0.4-1.0 mg/ml;
histidine hydrochloride monohydrate 0.08-4.2 mg/ml;
(iii) osmotic agent being trehalose 70-130.0 mg/ml;
(iv) water for injection to 1 ml;
or
(i) anti-TRBV9 antibody;
(ii) histidine buffer being a mixture of
histidine 0.4-1.0 mg/ml;
histidine hydrochloride monohydrate 0.08-4.2 mg/ml;
(iii) osmotic agent being sodium chloride 6.0-11.5 mg/ml;
(iv) water for injection to 1 ml.

74. The pharmaceutical composition as claimed in claim 33, comprising:
(i) anti-TRBV9 antibody;
(ii) histidine buffer being a mixture of
histidine 0.517 mg/ml;
histidine hydrochloride monohydrate 0.350 mg/ml;
(iii) osmotic agent being proline 27 mg/ml;
(iv) water for injection to 1 ml;
or
(i) anti-TRBV9 antibody;
(ii) histidine buffer being a mixture of
histidine 0.689 mg/ml
histidine hydrochloride monohydrate 0.117 mg/ml;
(iii) osmotic agent being proline 19 mg/ml;
(iv) water for injection to 1 ml;
or
(i) anti-TRBV9 antibody;
(ii) histidine buffer being a mixture of
histidine 0.517 mg/ml;
histidine hydrochloride monohydrate 0.350 mg/ml;
(iii) osmotic agent being sorbitol 50.0 mg/ml;
(iv) water for injection to 1 ml;
or
(i) anti-TRBV9 antibody;
(ii) histidine buffer being a mixture of
histidine 0.517 mg/ml;
histidine hydrochloride monohydrate 0.350 mg/ml;
(iii) osmotic agent being trehalose 100.0 mg/ml;
(iv) water for injection to 1 ml;
or
(i) anti-TRBV9 antibody;
(ii) histidine buffer being a mixture of
histidine 0.517 mg/ml;
histidine hydrochloride monohydrate 0.350 mg/ml;
(iii) osmotic agent being sodium chloride 9.0 mg/ml;
(iv) water for injection to 1 ml.

75. The pharmaceutical composition as claimed in claim 33, comprising:
(i) anti-TRBV9 antibody 25 mg/ml;
(ii) histidine buffer being a mixture of
histidine 0.517 mg/ml;
histidine hydrochloride monohydrate 0.350 mg/ml;
(iii) osmotic agent being proline 27 mg/ml;
(iv) water for injection to 1 ml.

76. The pharmaceutical composition as claimed in claim 49, comprising:
(i) anti-TRBV9 antibody;
(ii) histidine buffer or acetate buffer;
(iii) osmotic agent being proline 14.0-32.0 mg/ml;
(iv) stabilizer being glycine 5.5-9.5 mg/ml;
(v) water for injection to 1 ml;
or
(i) anti-TRBV9 antibody;
(ii) histidine buffer or acetate buffer;
(iii) osmotic agent being proline 14.0-32.0 mg/ml;
(iv) stabilizer being poloxamer 188 0.35-1.3 mg/ml;
(v) water for injection to 1 ml;
or
(i) anti-TRBV9 antibody;
(ii) histidine buffer or acetate buffer;
(iii) osmotic agent being sorbitol 35.0-65.0 mg/ml;
(iv) stabilizer being methionine 1.1-5.8 mg/ml;
(v) water for injection to 1 ml;
or
(i) anti-TRBV9 antibody;
(ii) histidine buffer or acetate buffer;
(iii) osmotic agent being sodium chloride 6.0-11.5 mg/ml;
(iv) stabilizer being poloxamer 188 or polysorbate 80 0.35-1.3 mg/ml;
(v) water for injection to 1 ml.

77. The pharmaceutical composition as claimed in claim 49, comprising:
(i) anti-TRBV9 antibody;
(ii) histidine buffer being a mixture of
histidine 0.4-1.0 mg/ml;
histidine hydrochloride monohydrate 0.08-4.2 mg/ml;
(iii) osmotic agent being proline 14.0-32.0 mg/ml;
(iv) stabilizer being glycine 5.5-9.5 mg/ml;
(v) water for injection to 1 ml;
or
(i) anti-TRBV9 antibody;
(ii) histidine buffer being a mixture of
histidine 0.4-1.0 mg/ml;
histidine hydrochloride monohydrate 0.08-4.2 mg/ml;
(iii) osmotic agent being proline 14.0-32.0 mg/ml;
(iv) stabilizer being poloxamer 188 0.35-1.3 mg/ml;
(v) water for injection to 1 ml;
or
(i) anti-TRBV9 antibody;
(ii) histidine buffer being a mixture of
histidine 0.4-1.0 mg/ml;
histidine hydrochloride monohydrate 0.08-4.2 mg/ml;
(iii) osmotic agent being sorbitol 35.0-65.0 mg/ml;
(iv) stabilizer being methionine 1.1-5.8 mg/ml;
(v) water for injection to 1 ml;
or
(i) anti-TRBV9 antibody;
(ii) histidine buffer being a mixture of
histidine 0.4-1.0 mg/ml;
histidine hydrochloride monohydrate 0.08-4.2 mg/ml;
(iii) osmotic agent being sodium chloride 6.0-11.5 mg/ml;
(iv) stabilizer being poloxamer 188 or polysorbate 80 0.35-1.3 mg/ml;
(v) water for injection to 1 ml.

78. The pharmaceutical composition as claimed in claim 49, comprising:
(i) anti-TRBV9 antibody;
(ii) histidine buffer being a mixture of
histidine 0.517 mg/ml;
histidine hydrochloride monohydrate 0.350 mg/ml;
(iii) osmotic agent being proline 21 mg/ml;
(iv) stabilizer being glycine 7.51 mg/ml;
(v) water for injection to 1 ml;
or
(i) anti-TRBV9 antibody;
(ii) histidine buffer being a mixture of
histidine 0.517 mg/ml
histidine hydrochloride monohydrate 0.350 mg/ml;
(iii) osmotic agent being proline 9.0 mg/ml;
(iv) stabilizer being poloxamer 188 0.5 mg/ml;
(v) water for injection to 1 ml;
or
(i) anti-TRBV9 antibody;
(ii) histidine buffer being a mixture of
histidine 0.517 mg/ml;
histidine hydrochloride monohydrate 0.350 mg/ml;
(iii) osmotic agent being proline 9.0 mg/ml;
(iv) stabilizer being poloxamer 188 1.0 mg/ml;
(v) water for injection to 1 ml;
or
(i) anti-TRBV9 antibody;
(ii) histidine buffer being a mixture of
histidine 0.517 mg/ml;
histidine hydrochloride monohydrate 0.350 mg/ml;
(iii) osmotic agent being sorbitol 50.0 mg/ml;
(iv) stabilizer being methionine 1.49 mg/ml;
(v) water for injection to 1 ml;
or
(i) anti-TRBV9 antibody;
(ii) histidine buffer being a mixture of
histidine 0.517 mg/ml;
histidine hydrochloride monohydrate 0.350 mg/ml;
(iii) osmotic agent being sorbitol 50.0 mg/ml;
(iv) stabilizer being methionine 4.48 mg/ml;
(v) water for injection to 1 ml;
or
(i) anti-TRBV9 antibody;
(ii) histidine buffer being a mixture of
histidine 0.517 mg/ml;
histidine hydrochloride monohydrate 0.350 mg/ml;
(iii) osmotic agent being sodium chloride 9.0 mg/ml;
(iv) stabilizer being poloxamer 188 or polysorbate 80 1.0 mg/ml;
(v) water for injection to 1 ml.

79. A pharmaceutical composition of anti-TRBV9 antibody as claimed in claim 1 for use in the treatment of a disease or disorder mediated by T-lymphocytes bearing a TRBV9 segment within the T cell receptor, in a subject in need thereof.

80. (canceled)

81. The pharmaceutical composition of anti-TRBV9 antibody as claimed in claim 79, wherein the disease or disorder mediated by T-lymphocytes bearing a TRBV9 segment within the T cell receptor is selected from the group comprising: spondyloarthritides, sacroiliitis associated with psoriasis, sacroiliitis associated with inflammatory bowel diseases, undifferentiated oligoarthropathy, juvenile spondylitis/enthesitis-related arthritis, juvenile ankylosing spondylitis (arthritis associated with enthesitis), juvenile arthritis, undifferentiated juvenile arthritis, ulcerative colitis, Crohn's disease, noninfectious uveitides, anterior uveitis, Behcet's disease, aortitis, fibrosis of aortic and/or mitral valve leaflets with regurgitation, rhythm disturbances, conduction disturbances, left ventricular dysfunction, pericarditis, myocarditis, IgA nephropathy, celiac disease, T cell lymphoma, T cell leukemia.

82. The pharmaceutical composition of anti-TRBV9 antibody as claimed in claim 81, wherein spondyloarthritis is radiographic axial spondyloarthritis (ankylosing spondylitis), axial spondyloarthritis, peripheral spondyloarthritis, psoriatic arthritis, spondyloarthritis associated with inflammatory bowel diseases, reactive arthritis, undifferentiated peripheral spondyloarthritis.
